(19) United States
(12) Patent Application Publication
(10) Pub. No.: US 2016/0331754 A1
(43) Pub. Date: Nov. 17, 2016

(54) THERAPIES FOR TREATING CANCERS

(71) Applicant: GILEAD SCIENCES, INC., Foster City, CA (US)

(72) Inventors: Roger Dansey, San Francisco, CA (US); Ronald L. Dubowy, Seattle, WA (US); Brian J. Lannutti, San Diego, CA (US); Sarah Meadows, Seattle, WA (US); Christophe Queva, Braine L'alleud (BE)

(21) Appl. No.: 15/112,060

(22) PCT Filed: Jan. 19, 2015
(86) PCT No.: PCT/US2015/011922

§ 371 Date: Jul. 15, 2016

Related U.S. Application Data
(60) Provisional application No. 62/088,422, filed on Dec. 5, 2014.
(60) Provisional application No. 61/929,370, filed on Jan. 20, 2014.
(60) Provisional application No. 62/046,881, filed on Sep. 5, 2014.

(51) Int. Cl. A61K 31/52; A61K 39/395; A61K 9/00; A61K 31/519

(52) U.S. Cl. CPC A61K 31/52; A61K 31/519; A61K 39/3955; A61K 9/0053; A61K 9/0019; A61K 2039/505

(57) ABSTRACT

Provided herein are methods, compositions, and kits for treating myeloproliferative disorders or neoplasms, including polycythemia vera, primary myelofibrosis, thrombocythemia, and essential thrombocythemia. Also provided herein are methods for treating cancers. Such methods may include the use of a JAK inhibitor and a PI3K inhibitor. Such methods may include the use of an anti-CD20 antibody and a PI3K inhibitor. Provided herein are also compositions, articles of manufacture and kits related thereto.

### FIELD

The present disclosure provides therapeutics and compositions for treating myeloproliferative disorders or neoplasms, and cancer, including, for example, leukemia, lymphoma, and multiple myeloma. The disclosure also provides the methods for preparation of the compositions, the articles of manufacture, and the kits thereof.

### BACKGROUND

Myeloproliferative disorders or neoplasms (MPN) are caused by genetic defects in the hematopoietic stem cells, resulting in clonal myeloproliferation, bone marrow fibrosis, and abnormal cytokine expression (Tefferi et al., Blood 108:1497-503, 2006). MPN may be classified into four subtypes: chronic myelogenous leukemia (CML), polycythemia vera (PV), essential thrombocythemia (ET), and primary myelofibrosis (PMF). Treatments of myeloproliferative disorders involve allogeneic stem cell transplant. The transplant procedure is preceded by myeloablative chemotherapy, can led to severe treatment-related consequence such as graft-versus-host disease and is limited by performance status, age and donor restrictions.

In 2005, a mutation JAK2V617F in Janus kinase 2 or JAK2, a member of the JAK family of kinases was identified (Baxter et al., Lancet 365:1054-61, 2005; James et al., Nature 434:1144-8, 2005; Kralovics et al., N. Engl. J. Med. 352:1779-90, 2005; Levine et al., Cancer Cell 7:387-97; 2005). The mutation constitutively activates JAK2 and JAK-STAT signaling, resulting in unrestrained cellular proliferation characteristics of myeloproliferative disorders. It is found in the subtypes of PV, ET, and PMF. About 99% of polycythemia vera patients and about 50-60% of essential thrombocytopenia patients and idiopathic myelofibrosis patients have the mutation JAK2V617F (Vainchenker et al., Blood 118:1723-35, 2011).

Several JAK inhibitors have been developed for treating myeloproliferative neoplasms, including ruxolitinib (INCB018424) for treating primary myelofibrosis, fedratinib (SAR302503, TG101348) for treating myelofibrosis, and XL019, SB1518 and AZD1480 for treating post-PV/ET myelofibrosis (Sonbol, Ther. Adv. Hematol. 4: 15-35, 2013). Patients treated with JAK inhibitors exhibit clinical improvement of reduced splenomegaly and/or constitutional symptoms. However, certain patients' anemia and thrombocytopenia conditions are aggravated. CYT387 (momelotinib) or N-(cyanomethyl)-4-(2-(4-morpholinophenylamino) pyrimidin-4-yl)benzamide is a different class of JAK inhibitor that provide additional benefits in improving anemia and/or spleen response. It is currently in clinical trials for treating primary myelofibrosis, polycythemia vera (PV), essential thrombocythemia (ET), and post-PV/ET.

The phosphatidylinositol 3-kinase (PI3K) pathway is shown to be dysregulated in certain myeloproliferative diseases (Kamishimoto et al., Cell Signaling 23: 849-56 2011; Huang et al., ASH 2009 Abstract 1896; Vannucchi et al., ASH 2011 Abstract 3835; Khan et al., Leukemia 27:1882-90, 2013). In vitro studies show that mTOR inhibitors, RAD001 or PP242, combined with AZD 1480 or ruxolitinib for 10-14 days resulted in reduced colony formation of erythropoietin endogenous erythroid cells from primary myelofibrosis or polycythemia vera patients (Bogani et al., PLOS One 8: e54826; 2013). Additional in vitro studies showed that JAK2 inhibitors, ruxolitinib or TG101348, combined with pan PI3K inhibitors ZSTK474, GDC0941, NVP-BEZ235, or LY294002 had synergistic effect (i.e. combination index less than 0.5) in reducing colony formation of cells from a polycythemia vera patient. However, no synergistic effect was detected for the combination of the JAK2 inhibitor ruxolitinib with TG100115 or the PI3Kδ inhibitor IC87114 (Choong et al., ASH 2012). There is no report on the effects of PI3K isoform inhibitors, such as PI3Kδ inhibitors, on myeloproliferative diseases.

It is shown that patients who have received chronic ruxolitinib treatment commonly develop disease persistence as shown by the gradual return of splenomegaly and/or constitutional symptoms, the lack of hematologic or molecular remissions, or the loss of clinical improvement (Gotlib, Hematologist, November 2012:11).

Accordingly, there is a need of effective treatment of myeloproliferative disorders including progressive or relapsed disease.

Similarly, cancer generally remains incurable with standard therapies. One example of such a cancer is chronic lymphocytic leukemia (CLL), a neoplasm resulting from the progressive accumulation of functionally incompetent monoclonal B lymphocytes in blood, bone marrow, lymph nodes, spleen, and liver.

In younger and relatively healthy patients with CLL, chemoimmunotherapy regimens that include the anti-CD20 monoclonal antibody, rituximab, are commonly employed to control disease manifestations (Gribben & O'Brien, J. Clin. Oncol. 2011; 29 (5):544-50). However, in elderly patients or patients with comorbid conditions, such regimens are associated with less efficacy and greater toxicity and increasing attention has been paid to the problem of treating patients with CLL who have comorbidities (Tam et al., Br. J. Haematol. 2008; 141 (1):36-40; Eichhorst et al., Leuk. Lymphoma, 2009; 50 (2):171-8; and Goede & Hallek, Drugs Aging 2011; 28 (3):163-76). Because of the relatively late age of diagnosis, a large proportion (˜90%) of patients with CLL have comorbidities and a substantial proportion (˜45%) have major chronic conditions such as coronary artery disease, diabetes, or chronic obstructive pulmonary disease. At the time the disease is first identified, ˜25% of patients with CLL do not meet conventional criteria for participation in clinical studies containing cytotoxic agents. (Thurmes et al., Leuk. Lymphoma 2008; 49 (1):49-56).

These health constraints in older or compromised patients have prompted noncytotoxic approaches to therapy. Alternative immunotherapeutics, such as the monoclonal antibodies, alemtuzumab or ofatumumab have been developed. (Keating et al., Blood 2002; 99 (10):3554-61; and Wierda et al., J. Clin. Oncol. 2010; 28 (10):1749-55). However, the therapeutic utility of the two drugs is modest; median progression-free survival (PFS) values in patients with recurrent CLL have been 4.7 months and 5.8 months, respectively. Moreover, these treatments can lead to other issues. For example, alemtuzumab can cause extreme immunosuppression that can lead to frequent opportunistic infection. Administration of the large amounts of protein recommended in product labeling for ofatumumab results in frequent infusion reactions and cumbersome infusion schedules.

In view of these conditions, repeated use of rituximab monotherapy or rituximab-corticosteroid combinations have been advocated in treatment guidelines for older or frail patients with recurrent CLL (Eichhorst et al., Ann. Oncol. 2010; 21 Suppl 5:v162-4; and Zelenetz et al., J. Natl. Compr. Canc. Netw. 2011; 9 (5):484-560). While single-agent rituximab use can offer certain benefits such as good tolerability in some patients with previously treated CLL, tumor control is not lasting, especially in patients with bulky adenopathy. (Gentile et al., Cancer management and research 2010; 2:71-81). Addition of high-dose methylprednisone to rituximab can extend median PFS to 12 months, but this combination is commonly associated with severe hyperglycemia and frequent life-threatening or fatal infections. See e.g., Bowen et al., Leuk Lymphoma 2007; 48 (12):2412-7; and Dungarwalla et al., Haematologica 2008; 93 (3):475-6.

As such, new noncytotoxic, well-tolerated, and convenient therapies are needed in order to enhance and prolong tumor control in patients with comorbid conditions. Due to the limitations of current treatments for cancer, there remains a significant interest in and need for additional or alternative therapies for treating, stabilizing, preventing, and/or delaying cancer.

### SUMMARY

In some aspects, provided herein are methods, compositions, articles of manufacture, and kits for treating a hyperproliferative disorder by using effective amounts of one, two or more therapeutic agents including a phosphatidylinositol 3-kinase delta (PI3Kδ) inhibitor, a Janus kinase (JAK) inhibitor, or the combination thereof. In some aspects, the methods described herein provide a treatment for a myeloproliferative disorder, comprising administering to a patient a therapeutic effective amount of JAK inhibitor and a therapeutic effective amount of PI3K inhibitor. In some aspects, the methods described herein provide a treatment for cancer, comprising administering to a patient a therapeutic effective amount of JAK inhibitor and a therapeutic effective amount of PI3K inhibitor.

In one aspect, the JAK inhibitor is selected from the group consisting of ruxolitinib, fedratinib, tofacitinib, baricitinib (INCB039110), lestaurtinib (CEP701), pacritinib (SB1518), XL019, AZD1480, gandotinib (LY2784544), BMS911543, fedratinib (SAR302503), decemotinib (V-509), INCB39110, GEN1, GEN2, GLPG0634, NS018, and N-(cyanomethyl)-4-[2-(4-morpholinoanilino)pyrimidin-4-yl]benzamide; or pharmaceutically acceptable salts thereof. In one embodiment, the JAK inhibit is ruxolitinib. In another embodiment, the JAK inhibitor is a JAK1/2 inhibitor such as N-(cyanomethyl)-4-[2-(4-morpholinoanilino)pyrimidin-4-yl]benzamide or a pharmaceutically acceptable salt thereof. In certain embodiments, the JAK inhibitor is a prodrug or solvate of one or more of the JAK inhibitors listed above.

In additional aspects, the PI3K inhibitor is selected from the group of XL147, BKM120, GDC-0941, BAY80-6946, PX-866, CH5132799, XL756, BEZ235, GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, ZSTK474, AS252424, TGX221, TG100115, IC87114, (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2-(1-((9H-purin-6-yl)amino)ethyl)-3-(2,6-difluorophenyl)quinazolin-4(3H)-one, and (S)-4-amino-6-((1-(5-chloro-4-oxo-3-phenyl-3,4-dihydroquinazolin-2-yl)ethyl)amino)pyrimidine-5-carbonitrile; or a pharmaceutically acceptable salt thereof. In certain embodiments, the PI3K inhibitor is a prodrug or solvate of one or more of the PI3K inhibitors listed above. In certain embodiments, the PI3K inhibitor is a PI3Kδ inhibitor selected from the group consisting of (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2-(1-((9H-purin-6-yl)amino)ethyl)-3-(2,6-difluorophenyl)quinazolin-4(3H)-one, and (S)-4-amino-6-((1-(5-chloro-4-oxo-3-phenyl-3,4-dihydroquinazolin-2-yl)ethyl)amino)pyrimidine-5-carbonitrile; or a pharmaceutically acceptable salt thereof. In certain embodiments, the PI3K inhibitor is a prodrug or solvate of S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2-(1-((9H-purin-6-yl)amino)ethyl)-3-(2,6-difluorophenyl)quinazolin-4(3H)-one, or (S)-4-amino-6-((1-(5-chloro-4-oxo-3-phenyl-3,4-dihydroquinazolin-2-yl)ethyl)amino)pyrimidine-5-carbonitrile

In certain aspects, the method comprises administering to a patient in need thereof N-(cyanomethyl)-4-[2-(4-morpholinoanilino) pyrimidin-4-yl]benzamide, or a pharmaceutically acceptable salt thereof, at a dose between 50 to 1000 mg, between 150 to 400 mg or between 100 mg to 800 mg. In some variations, the patient is a human subject. In other aspects, the method also comprises administering to a patient in need thereof with (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2-(1-((9H-purin-6-yl)amino)ethyl)-3-(2,6-difluorophenyl)quinazolin-4(3H)-one, or (S)-4-amino-6-((1-(5-chloro-4-oxo-3-phenyl-3,4-dihydroquinazolin-2-yl)ethyl)amino)pyrimidine-5-carbonitrile; or a pharmaceutically acceptable salt thereof at a dose between 100 mg and 1000 mg, between 125 mg and 400 mg, or between 150 mg and 800 mg. The JAK inhibitor may be administered prior to the PI3K inhibitor, concurrent with the PI3K inhibitor, or subsequent to the PI3K inhibitor. In some variations, the JAK inhibitor is administered orally, once or twice daily, in a form of tablet, pills, or capsules. Also, in some variations, the PI3K inhibitor is administered orally, once or twice daily, in a form of tablet, pills, or capsules.

In certain aspects, the method of treating myeloproliferative diseases further comprises one or more therapeutic agents, a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof. One or more therapeutic agent is selected from a PI3K (including PI3Kγ, PI3Kδ, PI3Kβ, PI3Kα, and/or pan-PI3K) inhibitor, a JAK (including JAK1 and/or JAK2) inhibitor, a SYK inhibitor, a BTK inhibitor, an A2B (adenosine A2B receptor) inhibitor, an ACK (activated CDC kinase, including ACK1) inhibitor, an ASK (apoptosis signal-regulating kinase, including ASK1) inhibitor, Auroa kinase, a BRD (bromodomain-containing protein, including BRD4) inhibitor, a CAK (CDK-activating kinase) inhibitor, a CaMK (calmodulin-dependent protein kinases) inhibitor, a CDK (cyclin-dependent kinases, including CDK1, 2, 3, 4, and/or 6) inhibitor, a CK (casein kinase, including CK1 and/or CK2) inhibitor, a DDR (discoidin domain receptor, including DDR1 and/or DDR2) inhibitor, a EGFR inhibitor, a FAK (focal adhesion kinase) inhibitor, a GSK (glycogen synthase kinase) inhibitor, a HDAC (histone deacetylase) inhibitor, an IDH (isocitrate dehydrogenase, including IDH1) inhibitor, an IKK inhibitor, a LCK (lymphocyte-specific protein tyrosine kinase) inhibitor, a LOX (lysyl oxidase) inhibitor, a LOXL (lysyl oxidase like protein, including LOXL1, LOXL2, LOXL3, LOXL4, and/or LOXL5) inhibitor, a MEK inhibitor, a matrix metalloprotease (MMP, including MMP2 and/or MMP9) inhibitor, a mitogen-activated protein kinases (MAPK) inhibitor, a PDGF (platelet-derived growth factor) inhibitor, a phosphorylase kinase (PK) inhibitor, a PLK (polo-like kinase, including PLK1, 2, 3) inhibitor, a protein kinase (PK, including protein kinase A, B, C) inhibitor, a serine/threonine kinase (STK) inhibitor, a STAT (signal transduction and transcription) inhibitor, a TBK (serine/threonine-protein kinase, including TBK1) inhibitor, a TK (tyrosine kinase) inhibitor, a TPL2 (serine/threonine kinase) inhibitor, a NEK9 inhibitor, an Abl inhibitor, a p38 kinase inhibitor, a PYK inhibitor, a PYK inhibitor, a c-Kit inhibitor, a NPM-ALK inhibitor, a Flt-3 inhibitor, a c-Met inhibitor, a KDR inhibitor, a TIE-2 inhibitor, a VEGFR inhibitor, a SRC inhibitor, a HCK inhibitor, a LYN inhibitor, a FYN inhibitor, and a YES inhibitor, or any combination thereof.

In some embodiments, the myeloproliferative disorder is selected from the group consisting of polycythemia vera (PV), primary myelofibrosis (PMF), thrombocythemia, essential thrombocythemia (ET), idiopathic myelofibrosis (IMF), chronic myelogenous leukemia (CML), systemic mastocystosis (SM), chronic neutrophilic leukemia (CNL), myelodysplastic syndrome (MDS) and systemic mast cell disease (SMCD).

In other aspects, a treatment is provided for patients having myeloproliferative disorder selected from the group consisting of polycythemia vera (PV), primary myelofibrosis (PMF), and essential thrombocythemia (ET). In some variations, the patient has received prior treatment and/or develops disease persistence to treatment of myeloproliferative disorder, or has not previously been treated for myeloproliferative disorder.

In another aspect, a method for decreasing cell viability, decreasing proliferation, or increasing apoptosis is provided. In some variations, such methods comprise contacting cells with an effective amount of JAK inhibitor and an effective amount of PI3K inhibitor. The JAK inhibitor is selected from the group consisting of ruxolitinib, fedratinib, tofacitinib, baricitinib, lestaurtinib, pacritinib, XL019, AZD1480, INCB039110, LY2784544, BMS911543, NS018, or N-(cyanomethyl)-4-[2-(4-morpholinoanilino)pyrimidin-4-yl]benzamide; or pharmaceutically acceptable salts thereof. Also, the PI3K inhibitor is selected from the group of XL147, BKM120, GDC-0941, BAY80-6946, PX-866, CH5132799, XL756, BEZ235, GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, ZSTK474, AS252424, TGX221, TG100115, IC87114, (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2-(1-((9H-purin-6-yl)amino)ethyl)-3-(2,6-difluorophenyl)quinazolin-4(3H)-one, (S)-4-amino-6-((1-(5-chloro-4-oxo-3-phenyl-3,4-dihydroquinazolin-2-yl)ethyl)amino)pyrimidine-5-carbonitrile; or a pharmaceutically acceptable salt thereof. In certain embodiments, the PI3K inhibitor is a prodrug or solvate of one of the agents listed above. The method uses cells that are isolated from a subject having myeloproliferative disorder selected from the group consisting of polycythemia vera, primary myelofibrosis, thrombocythemia, essential thrombocythemia, idiopathic myelofibrosis, chronic myelogenous leukemia, systemic mastocystosis, chronic neutrophilic leukemia, myelodysplastic syndrome, and systemic mast cell disease.

In other aspects, provided herein are also methods, compositions, articles of manufacture, and kits for treating a cancer by using effective amounts of agents selected from a PI3K inhibitor and an anti-CD20 antibody. In some variations, the PI3K inhibitor is a PI3Kδ inhibitor. In one variation, the PI3K inhibitor is Compound B:

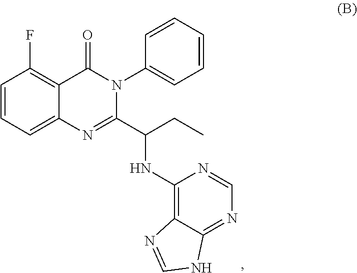 [SMILES: CCC(Nc1ncnc2[nH]cnc12)c1nc2cccc(F)c2c(=O)n1-c1ccccc1]

or a pharmaceutically acceptable salt thereof. In another variation, the PI3K inhibitor is Compound C:

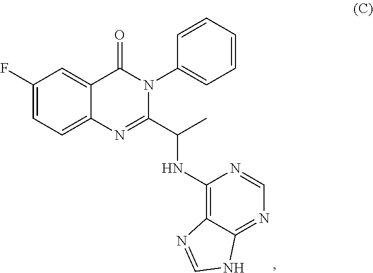 [SMILES: CC(Nc1ncnc2[nH]cnc12)c1nc2ccc(F)cc2c(=O)n1-c1ccccc1]

or a pharmaceutically acceptable salt thereof. In certain embodiments, the PI3K inhibitor is one a prodrug or solvate of Compound B or Compound C. In one embodiment, Compound B or Compound C, or a pharmaceutically acceptable salt, prodrug, or solvate thereof is predominantly the S-enantiomer.

Thus, in one aspect, provided is a method for treating a subject (e.g., a human), who has or is suspected of having a cancer, by administering to the subject in need of such treatment an effective amount of Compound B or Compound C, or a pharmaceutically acceptable salt thereof, and an effective amount of obinutuzumab. In one aspect, provided is a method for treating a subject (e.g., a human), who has or is suspected of having a cancer, by administering to the subject in need of such treatment an effective amount of a prodrug or solvate of Compound B or Compound C, and an effective amount of obinutuzumab.

In some embodiments, Compound B or Compound C or a pharmaceutically acceptable salt thereof is present in a pharmaceutical composition that includes Compound B or Compound C or a pharmaceutically acceptable salt thereof, and at least one pharmaceutically acceptable vehicle. In some embodiments, obinutuzumab is present in a pharmaceutical composition that includes Compound B or Compound C, and at least one pharmaceutically acceptable vehicle. In yet other embodiments, Compound B and obinutuzumab, or Compound C, or pharmaceutically acceptable salts thereof and obinutuzumab, are both present in a pharmaceutical composition that includes Compound B or Compound C, or pharmaceutically acceptable salts thereof, obinutuzumab, and at least one pharmaceutically acceptable vehicle.

In some embodiments, obinutuzumab, or a pharmaceutically acceptable salts thereof is administered before Compound B or a pharmaceutically acceptable salt thereof. In other embodiments, Compound B or Compound C, or a pharmaceutically acceptable salt thereof, and obinutuzumab, are administered simultaneously. In certain embodiments, each of Compound B and obinutuzumab, or each of Compound C and obinutuzumab, or a pharmaceutically acceptable salt thereof is independently administered once a day or twice a day.

In certain embodiments, the methods of the present disclosure comprise administering to a subject (e.g. a human) in need thereof Compound B or Compound C, or a pharmaceutically acceptable salt thereof, at a dose between 50 mg and 200 mg; and obinutuzumab at a dose between 100 mg and 750 mg. In certain embodiments, the dose of Compound B or Compound C or a pharmaceutically acceptable salt thereof is administered as one or more unit dosages each independently comprising between 50 mg and 200 mg of Compound B or Compound C or a pharmaceutically acceptable salt thereof; and the dose of obinutuzumab is administered as one or more unit dosages each independently comprising between 100 mg and 300 mg of obinutuzumab. In one embodiment, the dose of Compound B or Compound C or a pharmaceutically acceptable salt thereof is 100 mg or 150 mg; and the dose of obinutuzumab is 200 mg or 600 mg. In yet another embodiment, the dose of Compound B or Compound C or a pharmaceutically acceptable salt thereof is administered as a unit dosage comprising 100 mg or 150 mg of Compound B or Compound C or a pharmaceutically acceptable salt thereof; and the dose of obinutuzumab is administered as one or more unit dosages each independently comprising 25 mg, 100 mg or 200 mg of obinutuzumab. In some embodiments, the unit dosage is a tablet.

In some embodiments, Compound B, or a pharmaceutically acceptable salt thereof, and obinutuzumab are administered under fed conditions. In other embodiments, Compound C, or pharmaceutically acceptable salts thereof, and obinutuzumab are administered under fed conditions.

The anti-CD20 antibody may be administered prior to the PI3K inhibitor, concurrent with the PI3K inhibitor, or subsequent to the PI3K inhibitor. The anti-CD20 antibody may be administered intravenously. Also, the PI3K inhibitor may be administered orally, once or twice daily, in a form of tablet, pills, or capsules.

In other embodiments, the subject who has cancer is (i) refractory to at least one chemotherapy treatment, or (ii) is in relapse after treatment with chemotherapy, or a combination thereof. In certain embodiments, the subject has not previously been treated for the cancer. In one embodiment, the subject is a human subject.

In some embodiments, the cancer is Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML), mantle cell lymphoma (MCL), follicular lymphoma (FL), Waldestrom's macroglobulinemia (WM), T-cell lymphoma, B-cell lymphoma, diffuse large B-cell lymphoma (DLBCL), or marginal zone lymphoma (MZL) In certain embodiments, the cancer is leukemia, lymphoma, or multiple myeloma. In certain embodiments, the cancer is Burkitt's lymphoma, Hodgkin's lymphoma, non-Hodgkin's lymphoma, lymphocytic lymphoma, lymphocytic leukemia, multiple myeloma, or chronic myeloid leukemia. In one embodiment, the cancer chronic lymphocytic leukemia, B-cell acute lymphocytic leukemia, diffuse large B-cell lymphoma, or mantle cell lymphoma. In one embodiment, the cancer is minimal residual disease (MRD).

In particular embodiments, the cancer is leukemia or lymphoma. In specific embodiments, the cancer is acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML), multiple myeloma (MM), indolent non-Hodgkin's lymphoma (iNHL), refractory iNHL, non-Hodgkin's lymphoma (NHL), mantle cell lymphoma (MCL), follicular lymphoma, Waldestrom's macroglobulinemia (WM), T-cell lymphoma, B-cell lymphoma, and diffuse large B-cell lymphoma (DLBCL). In one embodiment, the cancer is T-cell acute lymphoblastic leukemia (T-ALL), or B-cell acute lymphoblastic leukemia (B-ALL). The non-Hodgkin lymphoma encompasses the indolent B-cell diseases that include, for example, follicular lymphoma, lymphoplasmacytic lymphoma, Waldenstrom macroglobulinemia, and marginal zone lymphoma, as well as the aggressive lymphomas that include, for example, Burkitt lymphoma, diffuse large B-cell lymphoma (DLBCL) and mantle cell lymphoma (MCL). In one embodiment, the leukemia is minimal residual disease (MRD).

In another aspects, provided is also a method for decreasing cell viability in cancer cells in a human, comprising administering to the human Compound B or Compound C or a pharmaceutically acceptable salt thereof, and obinutuzumab in amounts sufficient to detectably decrease cell viability in the cancer cells. Provided is also a method for decreasing cell viability in cancer cells, comprising contacting cancer cells with Compound B or Compound C or a pharmaceutically acceptable salt thereof, and obinutuzumab in amounts sufficient to detectably decrease cell viability in the cancer cells. In some embodiments, the cell viability in the cancer cells after administering to the human, or contacting the cancer cells with, Compound B or pharmaceutically acceptable salts thereof, and obinutuzumab, or with Compound C or pharmaceutically acceptable salts thereof, and obinutuzumab is decreased by at least 10% compared to cell viability in cancer cells after administering to the human, or contacting the cancer cells with, only Compound B or Compound C, or a pharmaceutically acceptable salt thereof or after administering to the human, or contacting the cancer cells with, only obinutuzumab. In one embodiment, cell viability in the cancer cells is determined by a cell viability assay, such as MTS assay.

Provided is also a method for decreasing AKT phosphorylation, S6 phosphorylation, or AKT and S6 phosphorylation in cancer cells in a human, comprising administering to the human Compound A or C or a pharmaceutically acceptable salt thereof, and obinutuzumab in amounts sufficient to detectably decrease AKT phosphorylation, S6 phosphorylation, or AKT and S6 phosphorylation in the cancer cells. Provided is also a method for decreasing AKT phosphorylation, S6 phosphorylation, or AKT and S6 phosphorylation in cancer cells, comprising contacting cancer cells with Compound A or C or a pharmaceutically acceptable salt thereof, and obinutuzumab in amounts sufficient to detectably decrease AKT phosphorylation, S6 phosphorylation, or AKT and S6 phosphorylation in the cancer cells. In some embodiments, S6 phosphorylation in the cancer cells after administering to the human, or contacting the cancer cells with, Compound B and obinutuzumab, or with Compound C and obinutuzumab, is decreased by at least 10% compared to S6 phosphorylation in cancer cells after administering to the human, or contacting the cancer cells with, only Compound B or Compound C, or a pharmaceutically acceptable salt thereof or after administering to the human, or contacting the cancer cells with, only obinutuzumab. In one embodiment, AKT phosphorylation, S6 phosphorylation, or AKT and S6 phosphorylation in the cancer cells is/are determined by flow cytometry. In certain embodiments, the cancer cells are chronic lymphocytic leukemia (CLL) cells.

In another aspect, provided is a method for decreasing AKT phosphorylation, ERK phosphorylation, or AKT and ERK phosphorylation in cancer cells in a human, comprising administering to the human Compound B or Compound C or a pharmaceutically acceptable salt thereof, and obinutuzumab in amounts sufficient to detectably decrease AKT phosphorylation, ERK phosphorylation, or AKT and ERK phosphorylation in the cancer cells. In another aspect, provided is a method for decreasing AKT phosphorylation, ERK phosphorylation, or AKT and ERK phosphorylation in cancer cells, comprising contacting cancer cells with Compound B or Compound C or a pharmaceutically acceptable salt thereof, and obinutuzumab in amounts sufficient to detectably decrease AKT phosphorylation, ERK phosphorylation, or AKT and ERK phosphorylation in the cancer cells. In some embodiments, ERK phosphorylation in the cancer cells after administering to the human, or contacting the cancer cells with, Compound B and obinutuzumab, or with Compound C and obinutuzumab, is decreased by at least 10% compared to ERK phosphorylation in cancer cells after administering to the human, or contacting the cancer cells with, only Compound B or Compound C, or a pharmaceutically acceptable salt thereof or after administering to the human, or contacting the cancer cells with, only obinutuzumab. In some embodiments, AKT phosphorylation, ERK phosphorylation, or AKT and ERK phosphorylation in the cancer cells is/are determined by immunoblotting. In one embodiment, the cancer cells are Burkitt's lymphoma cells.

In yet another aspect, provided is a method of decreasing chemokine production in a sample comprising cells expressing CCL2, CCL3, CCL4, CCL22, or any combinations thereof, comprising contacting the sample with Compound B or Compound C or a pharmaceutically acceptable salt thereof, and obinutuzumab in amounts sufficient to detectably chemokine production in the sample. In some embodiments, one or more of the following (i)-(iv) applies: (i) CLL2 production in the cells after contact with Compound B and obinutuzumab, or with Compound C and obinutuzumab, is decreased by at least 5% compared to CLL2 production in the cells after contact with only Compound B or Compound C, or a pharmaceutically acceptable salt thereof or after contact with only obinutuzumab; (ii) CLL3 production in the cells after contact with Compound B and obinutuzumab, with Compound C and obinutuzumab, is decreased by at least 5% compared to CLL3 production in the cells after contact with only Compound B or Compound C, or a pharmaceutically acceptable salt thereof or after contact with only obinutuzumab; (iii) CLL4 production in the cells after contact with Compound B and obinutuzumab, or with Compound C and obinutuzumab, is decreased by at least 5% compared to CLL4 production in the cells after contact with only Compound B or Compound C, or a pharmaceutically acceptable salt thereof or after contact with only obinutuzumab; and (iv) CLL22 production in the cells after contact with Compound B and obinutuzumab, or with Compound C, or pharmaceutically acceptable salts thereof, and obinutuzumab is decreased by at least 5% compared to CLL22 production after contact with only Compound B or Compound C, or a pharmaceutically acceptable salt thereof or after contact with only obinutuzumab. In one embodiment, the chemokine production in the sample is determined by an immunoassay.

In any of the foregoing embodiments related to the method for decreasing cell viability, decreasing AKT phosphorylation, S6 phosphorylation, or AKT and S6 phosphorylation, decreasing AKT phosphorylation, ERK phosphorylation, or AKT and ERK phosphorylation, and decreasing chemokine production in cells, the method may be performed in vitro, in vivo, or ex vivo. When the method is performed in vivo, in one aspect, the method comprises administering Compound B and obinutuzumab, or Compound C and obinutuzumab, to an a subject (e.g., a human) in need thereof.

In another aspect, provided is a method of sensitizing cancer cells in a human receiving a treatment of Compound B or Compound C or a pharmaceutically acceptable salt thereof, wherein the method comprises administering to the human obinutuzumab before or concurrently with treating the human with Compound B or Compound C, or a pharmaceutically acceptable salt thereof. In another aspect, provided is a method of sensitizing cancer cells receiving a treatment of Compound B or Compound C or a pharmaceutically acceptable salt thereof, wherein the method comprises contacting the cancer cells with obinutuzumab before or concurrently with treating the cancer cells with Compound B or Compound C, or a pharmaceutically acceptable salt thereof.

In yet another aspect, provided is a method of sensitizing a subject who is (i) substantially refractory to at least one chemotherapy treatment, or (ii) is in relapse after treatment with chemotherapy, or both (i) and (ii), wherein the method comprises administering to the subject an effective amount of Compound B or Compound C or a pharmaceutically acceptable salt thereof, and an effective amount of obinutuzumab.

In some aspects, a pharmaceutical composition is provided. In some variations, the pharmaceutical composition comprises a therapeutically effective amount of a JAK inhibitor, a therapeutically effective amount of PI3K inhibitor, and a pharmaceutically acceptable excipient. In other variations, a therapeutically effective amount of a PI3K inhibitor, a therapeutically effective amount of an anti-CD20 antibody, and a pharmaceutically acceptable excipient.

In certain aspects, a kit comprising a pharmaceutical composition and a label is provided. In some variations, the kit contains the pharmaceutical composition that comprises a therapeutically effective amount of a JAK inhibitor, a therapeutically effective amount of PI3K inhibitor, and a pharmaceutically acceptable excipient. In certain variations, the kit comprises: (i) a pharmaceutical composition comprising a JAK inhibitor, and at least one pharmaceutically acceptable vehicle; and (ii) a pharmaceutical composition comprising a PI3K inhibitor, and at least one pharmaceutically acceptable vehicle. In some embodiments, the kit further comprises: a package insert containing instructions for use of the pharmaceutical compositions in treating a myeloproliferative disorder. In other variations, the kit comprises: (i) a pharmaceutical composition comprising Compound B or Compound C or a pharmaceutically acceptable salt thereof, and at least one pharmaceutically acceptable vehicle; and (ii) a pharmaceutical composition comprising obinutuzumab, and at least one pharmaceutically acceptable vehicle. In some embodiments, the kit further comprises: a package insert containing instructions for use of the pharmaceutical compositions in treating a cancer. In one embodiment, each pharmaceutical composition is independently a tablet.

In certain aspects, an article of manufacture is provided. In one variation, the article of manufacture comprises: (i) a unit dosage form of a JAK inhibitor, and at least one pharmaceutically acceptable vehicle; (ii) a unit dosage form of a PI3K inhibitor; and at least one pharmaceutically acceptable vehicle; and (iii) a label containing instructions for use of the JAK inhibitor and the PI3K inhibitor in treating myeloproliferative disorder. In another variation, the article of manufacture comprises: (i) a unit dosage form of Compound B or Compound C or a pharmaceutically acceptable salt thereof, and at least one pharmaceutically acceptable vehicle; (ii) a unit dosage form of obinutuzumab; and at least one pharmaceutically acceptable vehicle; and (iii) a label containing instructions for use of Compound and obinutuzumab, or for use of Compound C and obinutuzumab, in treating cancer. In some embodiments, each unit dosage form is a tablet.

### DETAILED DESCRIPTION

In the following description, certain specific details are set forth in order to provide a thorough understanding of various embodiments of the disclosure. However, one skilled in the art will understand that the embodiments may be practiced without these details. The description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

The following description sets forth exemplary methods, compositions, kits and articles of manufacture for treating myeloproliferative disorders or neoplasm. Such description exemplifies embodiments and does not limit the scope of the present disclosure.

The present application provides methods for treating hyperproliferative disorders such as cancers and myeloproliferative disorders in a subject by administering one or more therapeutic agents. The myeloproliferative disorders (MPD), also referred to as myeloproliferative neoplasms (MPN), are caused by mutations in the hematopoietic (or early myeloid progenitor) stem cells that result in excessive production of myeloid lineage cells (such as bone marrow), clonal myeloproliferation, bone marrow fibrosis, and abnormal cytokine expression. MPN includes, among others, polycythemia vera (PV), primary myelofibrosis, thrombocythemia, essential thrombocythemia (ET), idiopathic myelofibrosis, chronic myelogenous leukemia (CML), systemic mastocystosis, chronic neutrophilic leukemia, myelodysplastic syndrome, and systemic mast cell disease. MPN patients may further develop acute myeloid leukemia (AML), which is often associated with a poor outcome. Current MPN therapies aim at providing palliative care over a long period of time.

The methods provided herein treat myeloproliferative diseases by administering one or more therapeutic agents for treating myeloproliferative diseases. In certain embodiments, the methods use or include a single therapeutic agent. In other embodiment, the methods use or include a combination of two or more therapeutic agents. In some embodiments, a method is provided for treating myeloproliferative diseases by administering a combination of therapeutic agents or small molecule inhibitors that inhibit B-cell receptor (BCR)-mediated signaling, phosphatidylinositol 3-kinase (PI3K)-mediated, Janus kinase (JAK)-mediated signaling pathways, or any combination thereof.

In other aspects, the methods provided herein treat a cancer in a subject by administering a combination of small molecule kinase inhibitors. The cancer may be a hematological malignancy, such as leukemia, lymphoma, or multiple myeloma. The subject may be a human. For example, in some embodiments, provided is a method for treating leukemia by administering a combination of small molecule kinase inhibitors that can inhibit B-cell receptor (BCR)-mediated signaling pathways and disrupt essential chronic lymphocytic leukemia (CLL) cell-microenvironment interactions. The methods provided herein may have the effect of inhibiting multiple nodes in the BCR pathway. Simultaneous inhibition of multiple pathways downstream of the BCR may result in a synergistic response that can help with overcoming the resistance observed with single compound use. Thus, dual inhibition may enhance antitumor effects in leukemia, including, for example, chronic lymphocytic leukemia (CLL).

A therapeutic agent may be a compound or a biologic molecule (such as DNA, RNA, or protein) that provide desired therapeutic effects when administered to a subject in need thereof (e.g. MPN patients). For example, the therapeutic agent is a compound that inhibits a kinase that, directly or indirectly, relates to the disease mechanism or development. As used herein, enhanced therapeutic effects or variants thereof refer to additional beneficial or synergistic effects to patients that are not observed previously, including fewer and/or reduced symptoms, higher survival rate, prolonged survival time, shorter treatment duration, lower drug dosage, increased molecular and/or cellular responses, and the like.

The combination of therapeutic agents or inhibitors may target upstream or downstream components of the same pathway. Alternatively, the combination of therapeutic agents or inhibitors may target different components of dual or multiple pathways. It is hypothesized that the use of a combination of therapeutic agents or inhibitors may enhance therapeutic effects compared to the use of a single therapeutic agent or inhibitor.

PI3K Class I has four p110 catalytic subunit isoforms α, β, δ, and γ. The PI3K p110 delta isoform is over-expressed in many B-cell malignancies, including CLL. It is shown that the PI3Kδ inhibitors promote apoptosis in B-cell malignancies by disrupting the molecular pathways related to BCR signaling, leukemia cell migration and microenvironment. Also, PI3Kδ inhibitors inhibit BCR derived PI3K signaling, which leads to inhibition of AKT activation. Without being bound to any theories, a PI3Kδ inhibitor may resensitize or reactivate JAK2 phosphorylation in the JAK-signaling pathway, resulting in increased patient response to prior, concurrent, or subsequent MPN therapies by overcoming drug resistance or disease persistence from the use of a single JAK inhibitor such as ruxolitinib. Alternatively, targeting PI3K p110δ inhibition may result in direct destruction of the diseased cell or repression of microenvironmental signals that are needed for signaling pathways relating to cell survival, proliferation, or hyperproliferation. As described herein, targeting or inhibiting PI3Kδ and JAK provides a novel approach for the treatment of hyperproliferative diseases.

Regardless of the mechanism, such effects are desired in treating hyperproliferative diseases such as cancers and MPN as the treatment is generally provided over a long period of time (i.e. chronic therapies) and drug resistance or disease persistence are commonly observed during chronic therapies. Thus, dual or multiple inhibitions by a combination of two, three or more therapeutic agents may enhance treatment or therapeutic effects in myeloproliferative diseases.

The disclosure also provides compositions (including pharmaceutical compositions, formulations, or unit dosages), articles of manufacture and kits comprising one or more therapeutic agents. In one aspect, provided are compositions (including pharmaceutical compositions, formulations, or unit dosages), articles of manufacture and kits comprising two or more agents selected from a JAK inhibitor, and a PI3K inhibitor. In another aspect, provided are compositions (including pharmaceutical compositions, formulations, or unit dosages), articles of manufacture and kits comprising two or more agents selected from a PI3Kδ inhibitor and an anti-CD20 antibody. For example, the two or more agents are two agents: (i) a PI3Kδ inhibitor, or a pharmaceutically acceptable salt thereof, and (ii) an humanized anti-CD20 monoclonal antibody.

As described in the present disclosure, in certain embodiments, the administration of a PI3Kδ inhibitor, including (S)-2-(1-(9H-purin-6-ylamino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2-(1-((9H-purin-6-yl)amino)ethyl)-3-(2,6-difluorophenyl)quinazolin-4(3H)-one, or (S)-4-amino-6-((1-(5-chloro-4-oxo-3-phenyl-3,4-dihydroquinazolin-2-yl)ethyl)amino)pyrimidine-5-carbonitrile, and a JAK inhibitor, including N-(cyanomethyl)-4-(2-((4-morpholinophenyl)amino)pyrimidin-4-yl)benzamide or ruxolitinib, to diseased cells or patients has led to unexpected enhanced therapeutic effects compared to the administration of each kinase inhibitor alone. The unexpected synergistic effects include, but are not limited to, for example, decreased cell viability, increased cell death or apoptosis, decreased inhibition or interference with PI3K signaling pathways (including AKT, S6RP, ERK phosphorylation), and/or reduction in chemokine (e.g., CCL2, CCL3, CLL4 and CLL22) production, reduced colony formation in diseased cells or patients. Further, the administration of both PI3Kδ and JAK inhibitors unexpectedly restored or increased sensitivity or response of the diseased cells that had developed resistance or the patients developed disease persistence to prior treatment.

As described in the present disclosure, in other embodiments, the administration of (S)-2-(1-(9H-purin-6-ylamino)propyl)-5-fluoro-3-phenylquinazolin-4(3H)-one (S-enantiomer of Compound B) or (S)-2-(1-((9H-purin-6-yl)amino)ethyl)-6-fluoro-3-phenylquinazolin-4(3H)-one (S-enantiomer of Compound C), which are each a PI3Kδ inhibitor, and obinutuzumab (e.g., GAZYVA®), which is a humanized anti-CD20 monoclonal antibody of the IgG1 subclass, to cancer cells results in synergistic effects compared to the administration of each compound alone. In certain embodiments, the unexpected synergistic effects include, but are not limited to, for example, decreased cell viability in cancer cells, inhibition or interference with BCR signaling pathways (including MEK and ERK phosphorylation), and/or reduction in chemokine production (e.g., CCL2, CCL3, CLL4 and CLL22 production). Further, in certain embodiments, the administration of both compounds to cancer cells restores sensitivity or response of such cancer cells that have developed resistance to either compound alone; or increases sensitivity or response of such cancer cells that developed resistance to either compound alone.

### Therapeutic Agents

The present application provides methods, compositions, kits and articles of manufacture thereof that use or include one or more therapeutic agents inhibiting one or more targets that relate to, directly or indirectly, to cell growth, proliferation, or apoptosis for treating hyperproliferative disorders such as cancers or myeloproliferative neoplasms. The one or more therapeutic agents are compounds or molecules that is an Abl inhibitor, an ACK inhibitor, an A2B inhibitor, an ASK inhibitor, an Auroa kinase inhibitor, a BTK inhibitor, a BRD inhibitor, a c-Kit inhibitor, a c-Met inhibitor, a CAK inhibitor, a CaMK inhibitor, a CDK inhibitor, a CK inhibitor, a DDR inhibitor, an EGFR inhibitor, a FAK inhibitor, a Flt-3 inhibitor, a FYN inhibitor, a GSK inhibitor, a HCK inhibitor, a HDAC inhibitor, an IKK inhibitor, an IDH inhibitor, an IKK inhibitor, a JAK inhibitor, a KDR inhibitor, a LCK inhibitor, a LOX inhibitor, a LOXL inhibitor, a LYN inhibitor, a MMP inhibitor, a MEK inhibitor, a MAPK inhibitor, a NEK9 inhibitor, a NPM-ALK inhibitor, a p38 kinase inhibitor, a PDGF inhibitor, a PI3 kinase (PI3K), a PK inhibitor, a PLK inhibitor, a PK inhibitor, a PYK inhibitor, a SYK inhibitor, a TPL2 inhibitor, a STK inhibitor, a STAT inhibitor, a SRC inhibitor, a TB K inhibitor, a TIE inhibitor, a TK inhibitor, a VEGF inhibitor, a YES inhibitor, a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof. In some embodiment, the therapeutic agents are compounds or molecules that target a PI3 kinase (PI3K), a spleen tyrosine kinase (SYK), a Janus kinase (JAK), a Bruton's tyrosine kinase (BTK), or any combination thereof, resulting in the inhibition of one or more targets. In certain embodiments, the therapeutic agent is a PI3Kδ inhibitor that selectively inhibits PI3K p110 delta isoform (PI3Kδ). In some embodiments, the therapeutic agents are a PI3Kδ inhibitor and a JAK1/2 inhibitor. In other embodiments, the therapeutic agents are a PI3K inhibitor and an immunotherapeutic agent. In certain embodiments, the therapeutic agents are a PI3Kδ inhibitor and an anti-CD20 antibody. In certain embodiments, the anti-CD20 antibody is obinutuzumab (GAZYVA®).

In certain embodiments, Compound B and C, or pharmaceutically acceptable salts thereof, alone or together, are administered in combination with an anti-CD20 antibody. In certain embodiments, the anti-CD20 antibody is a humanized anti-CD20 antibody. In certain embodiments, the anti-CD20 antibody is a monoclonal antibody. In certain embodiments, the anti-CD20 antibody is a humanized anti-CD20 monoclonal antibody. In certain embodiments, the anti-CD20 antibody is an antibody of the IgG1 subclass. In certain embodiments, the anti-CD20 antibody is a humanized anti-CD20 monoclonal antibody of the IgG1 subclass.

The JAK inhibitor binds and inhibits one or more members of JAK family, including JAKE JAK2, and/or JAK3. For example, the JAK inhibitor is the compound having the structure of formula (I) shown below.

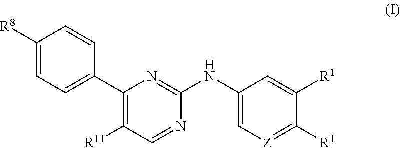

wherein

Z is independently selected from N and CH;

R¹ is independently selected from H, halogen, OH, CONHR², CON(R²)₂, CF₃, R²OR², CN, morpholino, thiomorpholinyl, thiomorpholino-1, 1-dioxide, optionally substituted piperidinyl, optionally substituted piperazinyl, imidazolyl, optionally substituted pyrrolidinyl and C₁₋₄alkylene wherein the carbon atoms are optionally substituted with NR^{Y} and/or O substituted with morpholino, thiomorpholinyl, thiomorpholino-1,1-dioxide, optionally substituted piperidinyl, optionally substituted piperazinyl, imidazolyl or optionally substituted pyrrolidinyl;

R² is optionally substituted C₁₋₄alkyl;

R^{Y} is H or optionally substituted C₁₋₄alkyl;

R⁸ is R^{X}CN;

R^{X} is optionally substituted C₁₋₄alkylene wherein up to 2 carbon atoms can be optionally substituted with CO, NSO₂R¹, NR^{Y}, CONR^{Y}, SO, SO₂ or O; and

R¹¹ is H, halogen, C₁₋₄alkyl or C₁₋₄alkyloxy;

or a pharmaceutically acceptable salt thereof.

In one embodiment, the JAK inhibitor is Compound A having the structure:

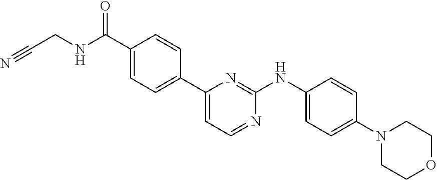 [SMILES: N#CCNC(=O)c1ccc(-c2ccnc(Nc3ccc(N4CCOCC4)cc3)n2)cc1]

Compound A may be referred to by its compound name: N-(cyanomethyl)-4-[2-(4-morpholinoanilino)pyrimidin-4-yl]benzamide using ChemDraw. Compound A, also referred to as CYT0387 or momelotinib, is a selective inhibitor to JAK2 and JAK1, relative to JAK3. Methods for synthesizing compounds of formula I and Compound A are previously described in U.S. Pat. No. 8,486,941. This reference is hereby incorporated herein by reference in its entirety.

Additional JAK inhibitors include, but are not limited to, ruxolitinib (INCB018424), fedratinib (SAR302503, TG101348), tofacitinib, baricitinib, lestaurtinib, pacritinib (SB1518), XL019, AZD1480, INCB039110, LY2784544, BMS911543, and NS018.

The PI3K inhibitors inhibit one or more isoforms of Class I PI3K, including PI3Kα, PI3Kβ, PI3Kδ, PI3Kγ, or any combination thereof. For example, the PI3K inhibitor is a PI3Kδ inhibitor having the structure of formula II as shown below.

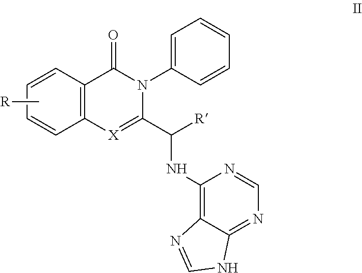

wherein

X is CH or N;

R is H, halo, or C₁₋₆ alkyl; and

R′ is C₁₋₆ alkyl;

or a pharmaceutically acceptable salt thereof.

In some embodiments, the PI3Kδ inhibitor is Compound B having the structure:

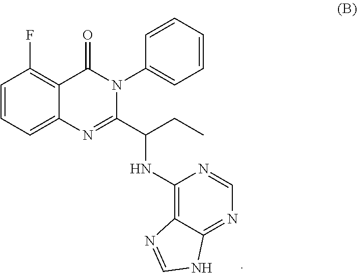 [SMILES: CCC(Nc1ncnc2[nH]cnc12)c1nc2cccc(F)c2c(=O)n1-c1ccccc1]

In other embodiments, Compound B is predominantly the S-enantiomer, having the structure:

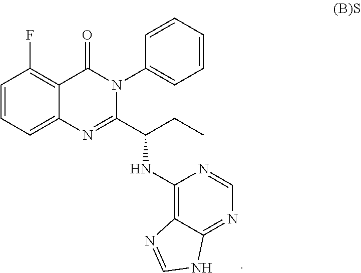 [SMILES: CC[C@H](Nc1ncnc2[nH]cnc12)c1nc2cccc(F)c2c(=O)n1-c1ccccc1]

The (S)-enantiomer of Compound B may also be referred to by its compound name: (S)-2-(1-((9H-purin-6-yl)amino)propyl)-5-fluoro-3-phenylquinazolin-4(3H)-one using ChemDraw.

In certain embodiments, the PI3Kδ inhibitor is Compound C having the structure:

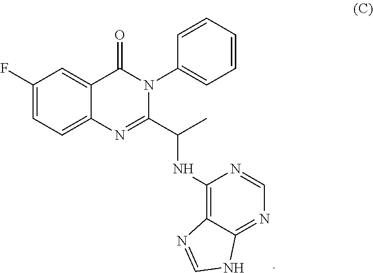 [SMILES: CC(Nc1ncnc2[nH]cnc12)c1nc2ccc(F)cc2c(=O)n1-c1ccccc1]

In additional embodiments, Compound C is predominantly the S-enantiomer, having the structure:

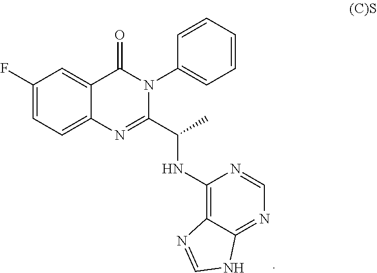 [SMILES: C[C@H](Nc1ncnc2[nH]cnc12)c1nc2ccc(F)cc2c(=O)n1-c1ccccc1]

The (S)-enantiomer of Compound C may also be referred to by its compound name: (S)-2-(1-((9H-purin-6-yl)amino)ethyl)-6-fluoro-3-phenylquinazolin-4(3H)-one using ChemDraw.

In another embodiment, the PI3K inhibitor is Compound D, having the structure:

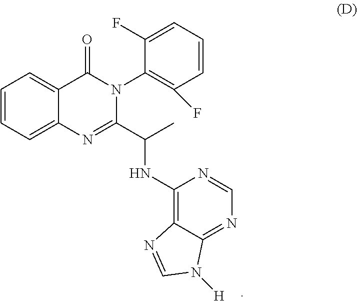 [SMILES: CC(Nc1ncnc2[nH]cnc12)c1nc2ccccc2c(=O)n1-c1c(F)cccc1F]

In one embodiment, Compound D is predominantly the S-enantiomer, having the structure:

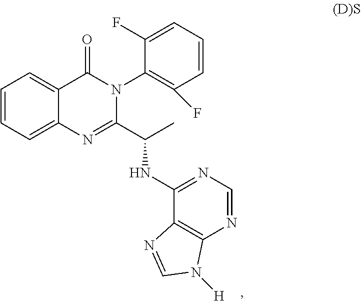 [SMILES: C[C@H](Nc1ncnc2[nH]cnc12)c1nc2ccccc2c(=O)n1-c1c(F)cccc1F]

The (S)-enantiomer of Compound D may also be referred to by its compound name: (S)-2-(1-((9H-purin-6-yl)amino)ethyl)-3-(2,6-difluorophenyl)quinazolin-4(3H)-one using ChemDraw.

In yet other embodiment, the PI3K inhibitor is Compound E which is named by its compound name: (S)-4-amino-6-((1-(5-chloro-4-oxo-3-phenyl-3,4-dihydroquinazolin-2-yl)ethyl)amino)pyrimidine-5-carbonitrile using ChemDraw. In some other embodiment, the PI3K inhibitor includes the compounds described in U.S. Provisional Application Nos. 61/543,176; 61/581,528; 61/745,429; 61/745,437; and 61/835,333. The references are hereby incorporated herein by reference in their entirety.

Compounds B, C, D, and E are PI3Kδ inhibitors, selectively inhibiting PI3K p110δ compared to other PI3K isoforms. Methods for synthesizing the compounds of formula II, Compounds B, C, D, and E are previously described in U.S. Pat. No. 7,932,260 or U.S. Provisional Application No. 61/581,528. The references are hereby incorporated herein by reference in their entirety, and specifically with respect to the synthesis of the compounds of formula II, Compounds B, C, D, and E.

Additional PI3K inhibitors include but are not limited to XL147, BKM120, GDC-0941, BAY80-6946, PX-866, CH5132799, XL756, BEZ235, and GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, AS252424, TGX221, TG100115, IC87114, and ZSTK474. In one variation, the PI3K inhibitor is duvelisib (IPI-145).

The SYK inhibitor includes but is not limited to 6-(1H-indazol-6-yl)-N-(4-morpholinophenyl)imidazo[1,2-a]pyrazin-8-amine, R406 (tamatinib), R788 (fostamatinib), PRT062607, BAY-61-3606, NVP-QAB 205 AA, R112, or R343, or a pharmaceutically acceptable salt thereof. See Kaur et al., European Journal of Medicinal Chemistry 67 (2013) 434-446. In one embodiment, the Syk inhibitor is 6-(1H-indazol-6-yl)-N-(4-morpholinophenyl)imidazo[1,2-a]pyrazin-8-amine as described in U.S. Pat. No. 8,450,321.

One skilled in the art understands that the compound structures may be named or identified using commonly recognized nomenclature systems and symbols. By way of example, the compound may be named or identified with common names, systematic or non-systematic names. The nomenclature systems and symbols that are commonly recognized in the art of chemistry include, for example, ChemBioDraw Ultra 12.0, Chemical Abstract Service (CAS) and International Union of Pure and Applied Chemistry (IUPAC). For example, the chemical name of Compound A may be referred to as N-(cyanomethyl)-4-[2-(4-morpholinoanilino) pyrimidin-4-yl]benzamide using ChemDraw 2.0 or N-(cyanomethyl)-4-(2-((4-morpholinophenyl)amino)pyrimidin-4-yl)benzamide using IUPAC, and the chemical name of Compound B may be referred to as (S)-2-(1-((9H-purin-6-yl)amino)propyl)-5-fluoro-3-phenylquinazolin-4(3H)-one using ChemDraw 2.0 or (5-Fluoro-3-phenyl-2-[(1S)-1-(9H-purin-6-ylamino)propyl]quinazolin-4(3H)-one) using IUPAC.

The term “selective inhibitor,” “selectively inhibits,” or variants refer to a compound or molecule that inhibits a member or isoform within the same protein family more effectively than at least one other member or isoform of the family. For example, the “PI3Kδ inhibitor” refers to a compound that inhibits the PI3Kδ isoform more effectively than at least one other isomers of the PI3K family, and the “JAK1/2 inhibitor” refers to a compound that inhibits JAK1/2 more effectively than at least one other members of the JAK family. The selective inhibitor may also be active against other members or isomers of the family, but requires higher concentrations to achieve the same degree of inhibition. “Selective” can also be used to describe a compound that inhibits a particular protein or kinase more so than a comparable compound.

The term “C₁₋₄alkyl” refers to straight chain or branched chain hydrocarbon groups having from 1 to 4 carbon atoms. Examples include methyl, ethyl, propyl, isopropyl, butyl, isobutyl, sec-butyl, and tert-butyl. Similarly, the term “C₁₋₆alkyl” refers to straight chain or branched chain hydrocarbon groups having from 1 to 6 carbon atoms

The term “halogen” refers to fluorine, chlorine, bromine and iodine.

The term “optionally substituted” refers to a group that is either unsubstituted or substituted with one or more groups selected from C₁₋₄ alkyl, C₃₋₆ cycloalkyl, C₂₋₆ alkenyl, C₂₋₆ alkynyl, C₁₋₆ alkylaryl, aryl, heterocyclyl, halo, haloC₁₋₆alkyl, haloC₃₋₆cycloalkyl, halo_{C2-6}alkenyl, haloC₂₋₆alkynyl, haloaryl, haloheterocyclyl, hydroxy, C₁₋₆ alkoxy, C₂₋₆alkenyloxy, C₂₋₆alkynyloxy, aryloxy, heterocyclyloxy, carboxy, haloC₁₋₆alkoxy, haloC₂₋₆alkenyloxy, haloC₂₋₆alkynyloxy, haloaryloxy, nitro, nitroC₁₋₆,alkyl, nitroC₂₋₆alkenyl, nitroaryl, nitroheterocyclyl, azido, amino, C₁₋₆alkylamino, C₂₋₆alkenylamino, C₂₋₆alkynylamino, arylamino, heterocyclamino acyl, C₁₋₆alkylacyl, C₂₋₆alkenylacyl, C₂₋₆alkynylacyl, arylacyl, heterocyclylacyl, acylamino, acyloxy, aldehydo, _{C1-6}alkylsulphonyl, arylsulphonyl, C₁₋₆alkylsulphonylamino, arylsulphonylamino, C₁₋₆alkylsulphonyloxy, arylsulphonyloxy, C₁₋₆alkylsulphenyl, C₂₋₆alklysulphenyl, arylsulphenyl, carboalkoxy, carboaryloxy, mercapto, C₁₋₆alkylthio, arylthio, acylthio, cyano and the like. In certain embodiments, “optionally substituted” refers to a group that is either unsubstituted or substituted with one or more groups selected from the group consisting of C₁₋₄ alkyl, C₃₋₆ cycloalkyl, C₂₋₆ alkenyl, C₂₋₆ alkynyl, C₁₋₆ alkylaryl, aryl, heterocyclyl, halo, haloaryl, haloheterocyclyl, hydroxy, C₁₋₄ alkoxy, aryloxy, carboxy, amino, C₁₋₆alkylacyl, arylacyl, heterocyclylacyl, acylamino, acyloxy, C₁₋₆alkylsulphenyl, arylsulphonyl and cyano.

The term “aryl” refers to single, polynuclear, conjugated or fused residues of aromatic hydrocarbons. Examples include phenyl, biphenyl, terphenyl, quaterphenyl, naphthyl, tetrahydronaphthyl, anthracenyl, dihydroanthracenyl, benzanthracenyl, dibenxanthracenyl and phenanthrenyl.

The term “unsaturated N-containing 5 or 6-membered heterocyclyl” refers to unsaturated, cyclic hydrocarbon groups containing at least one nitrogen. Suitable N-containing heterocyclic groups include unsaturated 5 to 6-membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl or tetrazolyl; unsaturated 5 or 6-membered heteromonocyclic group containing 1 to 2 oxygen atoms and 1 to 3 nitrogen atoms, such as, oxazolyl, isoxazolyl or oxadiazolyl; and unsaturated 5 or 6-membered heteromonocyclic group containing 1 to 2 sulphur atoms and 1 to 3 nitrogen atoms, such as, thiazolyl or thiadiazolyl.

The methods, compositions, kits and articles of manufacture provided herein use or include compounds (e.g., Compound A, Compound B, Compound C, Compound D, and Compound E) or pharmaceutically acceptable salts, prodrugs, or solvates thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of compounds or pharmaceutically acceptable salts, prodrugs, or solvates thereof, when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

As used herein, by “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” include, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, mesylate, bismesylate, benzoate, salicylate, p-toluenesulfonate, 2-hydroxyethylsulfonate, stearate, and alkanoate (such as acetate, HOOC—(CH₂)ₙ—COOH where n is 0-4). In addition, the compounds described herein may be obtained as an acid addition salt, and the free base may be obtained by basifying a solution of the acid salt. Alternatively, the product may be a free base, an addition salt including a pharmaceutically acceptable addition salt may be produced by dissolving the free base in a suitable organic solvent and treating the solution with an acid, in accordance with commonly known procedures for preparing acid addition salts from base compounds. Those skilled in the art will recognize various synthetic methods that may be used to prepare nontoxic pharmaceutically acceptable addition salts.

A “prodrug” includes any compound that becomes Compounds A, B, C, D, or E when administered to a subject, e.g., upon metabolic processing of the prodrug.

A “solvate” is formed by the interaction of a solvent and a compound. The compounds used in the methods and compositions (including, for example, pharmaceutical compositions, articles of manufacture and kits) may use or include solvates of salts of Compound A, Compound B, Compound C, Compound D, or Compound E. In one embodiment, the solvent may be hydrates of Compound A, Compound B, Compound C, Compound D, or Compound E.

The methods, compositions, kits and articles of manufacture provided may use or include optical isomers, racemates, or other mixtures thereof, of Compound B, Compound C, Compound D, or Compound E or a pharmaceutically acceptable salt, prodrug, or solvate thereof. The single enantiomer or diastereomer, i.e., optically active form, may be obtained by asymmetric synthesis or by resolution of the racemate. Resolution of racemates may be accomplished, for example, by known methods such as crystallization in the presence of a resolving agent, or chromatography, using, for example a chiral high pressure liquid chromatography (HPLC) column. In addition, provided are also Z- and E-forms (or cis- and trans-forms) of Compounds B, C, D, or E, or a pharmaceutically acceptable salt, prodrug, or solvate thereof with carbon-carbon double bonds. The methods, compositions, kits and articles of manufacture provided may use or include any tautomeric form of Compounds B, C, D, or E, or a pharmaceutically acceptable salt, prodrug, or solvate thereof.

In some embodiments, the methods, compositions, kits and articles of manufacture provided herein may use or include a racemic mixture, or a mixture containing an enantiomeric excess (e.e.) of one enantiomer of Compound B, Compound C, Compound D, or Compound E. All such isomeric forms of Compounds B, C, D, or E are included herein the same as if each and every isomeric form were specifically and individually listed. For example, Compound B, Compound C, Compound D, or Compound E has an enantiomeric excess of at least 60%, at least 65%, at least 70%, at least 75%, at least 80%, at least 85%, at least 90%, at least 95%, at least 98%, or at least 99% of its (S)-enantiomer.

By way of example, the methods, compositions, kits and articles of manufacture provided may use or include: (i) a mixture containing an enantiomeric excess of the (S)-enantiomer of Compound B, Compound C, Compound D, or Compound E or a pharmaceutically acceptable salt thereof; and (ii) Compound A, or ruxolitinib or a pharmaceutically acceptable salt thereof. In some embodiments, the methods, compositions, kits and articles of manufacture provided herein use or include Compound B or a pharmaceutically acceptable salt thereof, in an enantiomeric excess of the (S)-enantiomer, and Compound A or a pharmaceutically acceptable salt thereof.

For another example, in certain embodiments of the methods, compositions, kits and articles of manufacture provided may use or include: (i) a mixture containing an enantiomeric excess of the (S)-enantiomer of Compound B or Compound C, or a pharmaceutically acceptable salt thereof; and (ii) obinutuzumab. In other embodiments of the methods, compositions, kits and articles of manufacture provided may use or include: (i) a mixture containing an enantiomeric excess of the (S)-enantiomer of Compound B or Compound C; and (ii) obinutuzumab.

In some embodiment, the one or more therapeutic agents include inhibitors that are being used and/or developed to treat various hyperproliferative disorders such as cancer or myeloproliferative neoplasms. Exemplified therapeutic agents include compounds or molecules inhibiting pathways related to BCR, PI3K, SYK, and JAK, such as the agents inhibiting the RAS/RAF/MEK/ERK pathway, the PI3K/PTEN/AKT/mTOR pathway, the JAK-STAT pathway, either the entire or part of the pathway Inhibitors of mTOR include temsirolimus, everolimus, ridaforolimus (or deforolimus), OSI-027, AZD2014, CC-223, RAD001, LY294002, BEZ235, rapamycin, Ku-0063794, or PP242 Inhibitors of AKT include MK-2206, GDC-0068 and GSK795 Inhibitors of MEK include trametinib, selumetinib, cobimetinib, MEK162, PD-325901, PD-035901, AZD6244, and CI-1040. The application also uses and includes other inhibitors, such as CDK inhibitors (AT-7519, SNS-032), JNK inhibitors (CC-401), MAPK inhibitors (VX-702, SB203580, SB202190), Raf inhibitors (PLX4720), ROCK inhibitors (Rho-15), Tie2 inhibitors (AMG-Tie2-1), TK inhibitors (erlotinib), or any combination thereof. As described herein, such inhibitors include compounds or agents that inhibit all subclasses (e.g. isoforms or members) of a target (e.g. PI3K alpha, beta, delta and gamma) or a pathway, compounds or agents that inhibit primarily one subclass, and compounds or agents that inhibit a subset of all subclasses.

In the present application, the one or more therapeutic agents, including the PI3K inhibitor and/or JAK inhibitor, may be used or combined with a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof.

Chemotherapeutic agents may be categorized by their mechanism of action into, for example, the following groups: anti-metabolites/anti-cancer agents, such as pyrimidine analogs (floxuridine, capecitabine, and cytarabine); purine analogs, folate antagonists and related inhibitors antiproliferative/antimitotic agents including natural products such as vinca alkaloid (vinblastine, vincristine) and microtubule such as taxane (paclitaxel, docetaxel), vinblastin, nocodazole, epothilones and navelbine, epidipodophyllotoxins (etoposide, teniposide); DNA damaging agents (actinomycin, amsacrine, busulfan, carboplatin, chlorambucil, cisplatin, cyclophosphamide, Cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, etoposide, triethylenethiophosphoramide); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards cyclophosphamide and analogs, melphalan, chlorambucil), and (hexamethylmelamine and thiotepa), alkyl nitrosoureas (BCNU) and analogs, streptozocin), trazenes-dacarbazinine (DTIC); antiproliferative/antimitotic antimetabolites such as folic acid analogs (methotrexate); platinum coordination complexes (cisplatin, oxiloplatinim, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (breveldin); immunosuppressives tacrolimus sirolimus azathioprine, mycophenolate; compounds (TNP-470, genistein) and growth factor inhibitors (vascular endothelial growth factor inhibitors, fibroblast growth factor inhibitors); angiotensin receptor blocker, nitric oxide donors; anti-sense oligonucleotides; antibodies (trastuzumab, rituximab); cell cycle inhibitors and differentiation inducers (tretinoin); inhibitors, topoisomerase inhibitors (doxorubicin (adriamycin), daunorubicin, dactinomycin, eniposide, epirubicin, etoposide, idarubicin, irinotecan and mitoxantrone, topotecan, irinotecan), corticosteroids (cortisone, dexamethasone, hydrocortisone, methylpednisolone, prednisone, and prednisolone); growth factor signal transduction kinase inhibitors; dysfunction inducers, toxins such as Cholera toxin, ricin, Pseudomonas exotoxin, Bordetella pertussis adenylate cyclase toxin, or diphtheria toxin, and caspase activators; and chromatin.

As used herein the term “chemotherapeutic agent” or “chemotherapeutic” (or “chemotherapy,” in the case of treatment with a chemotherapeutic agent) is meant to encompass any non-proteinaceous (i.e, non-peptidic) chemical compound useful in the treatment of cancer. Examples of chemotherapeutic agents include alkylating agents such as thiotepa and cyclophosphamide (CYTOXAN®); alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammaII and calicheamicin phiI1, see, e.g., Agnew, Chem. Intl. Ed. Engl, 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; PSK; razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g., paclitaxel (TAXOL®, Bristol Meyers Squibb Oncology, Princeton, N.J.) and docetaxel (TAXOTERE®, Rhone-Poulenc Rorer, Antony, France); chlorambucil; gemcitabine (Gemzar®); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine®); novantrone; teniposide; edatrexate; daunomycin; aminopterin; xeoloda; ibandronate; CPT-11; topoisomerase inhibitor RFS 2000; difluoromethylomithine (DMFO); retinoids such as retinoic acid; capecitabine; FOLFIRI (fluorouracil, leucovorin, and irinotecan) and pharmaceutically acceptable salts, acids or derivatives of any of the above. One or more chemotherapeutic agent are used or included in the present application. For example, gemcitabine, nab-paclitaxel, and gemcitabine/nab-paclitaxel are used with the JAK inhibitor and/or PI3Kδ inhibitor for treating hyperproliferative disorders.

Also included in the definition of “chemotherapeutic agent” are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston®); inhibitors of the enzyme aromatase, which regulates estrogen production in the adrenal glands, such as, for example, 4(5)-imidazoles, aminoglutethimide, megestrol acetate (Megace®), exemestane, formestane, fadrozole, vorozole (Rivisor®), letrozole (Femara®), and anastrozole (Arimidex®); and anti-androgens such as flutamide, nilutamide, bicalutamide, leuprohde, and goserelin; and pharmaceutically acceptable salts, acids or derivatives of any of the above.

The anti-angiogenic agents include, but are not limited to, retinoid acid and derivatives thereof, 2-methoxyestradiol, ANGIOSTATIN®, ENDOSTATIN®, suramin, squalamine, tissue inhibitor of metalloproteinase-1, tissue inhibitor of metalloproternase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,I-3,4-dehydroproline, thiaproline, .alpha.-dipyridyl, beta-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, beta-cyclodextrin tetradecasulfate, eponemycin; fumagillin, gold sodium thiomalate, d-penicillamine (CDPT), beta-1-anticollagenase-serum, alpba-2-antiplasmin, bisantrene, lobenzarit disodium, n-2-carboxyphenyl-4-chloroanthronilic acid disodium or “CCA”, thalidomide; angiostatic steroid, cargboxynaminolmidazole; metalloproteinase inhibitors such as BB94. Other anti-angiogenesis agents include antibodies, preferably monoclonal antibodies against these angiogenic growth factors: beta-FGF, alpha-FGF, FGF-5, VEGF isoforms, VEGF-C, HGF/SF and Ang-1/Ang-2. See Ferrara N. and Alitalo, K. “Clinical application of angiogenic growth factors and their inhibitors” (1999) Nature Medicine 5:1359-1364.

The anti-fibrotic agents include, but are not limited to, the compounds such as beta-aminoproprionitrile (BAPN), as well as the compounds disclosed in U.S. Pat. No. 4,965,288 to Palfreyman, et al., issued Oct. 23, 1990, entitled “Inhibitors of lysyl oxidase,” relating to inhibitors of lysyl oxidase and their use in the treatment of diseases and conditions associated with the abnormal deposition of collagen; U.S. Pat. No. 4,997,854 to Kagan, et al., issued Mar. 5, 1991, entitled “Anti-fibrotic agents and methods for inhibiting the activity of lysyl oxidase in situ using adjacently positioned diamine analogue substrate,” relating to compounds which inhibit LOX for the treatment of various pathological fibrotic states, which are herein incorporated by reference. Further exemplary inhibitors are described in U.S. Pat. No. 4,943,593 to Palfreyman, et al., issued Jul. 24, 1990, entitled “Inhibitors of lysyl oxidase,” relating to compounds such as 2-isobutyl-3-fluoro-, chloro-, or bromo-allylamine; as well as, e.g., U.S. Pat. No. 5,021,456; U.S. Pat. No. 5,5059,714; U.S. Pat. No. 5,120,764; U.S. Pat. No. 5,182,297; U.S. Pat. No. 5,252,608 (relating to 2-(1-naphthyloxymemyl)-3-fluoroallylamine); and U.S. Patent Application No. 2004/0248871, which are herein incorporated by reference. Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives, semicarbazide, and urea derivatives, aminonitriles, such as beta-aminopropionitrile (BAPN), or 2-nitroethylamine, unsaturated or saturated haloamines, such as 2-bromo-ethylamine, 2-chloroethylamine, 2-trifluoroethylamine, 3-bromopropylamine, p-halobenzylamines, selenohomocysteine lactone. Also, the anti-fibrotic agents are copper chelating agents, penetrating or not penetrating the cells. Exemplary compounds include indirect inhibitors such compounds blocking the aldehyde derivatives originating from the oxidative deamination of the lysyl and hydroxylysyl residues by the lysyl oxidases, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, sodium-4-mercaptobutanesulphinate trihydrate.

The immunotherapeutic agents include and are not limited to therapeutic antibodies suitable for treating patients; such as abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomabn, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49 and 3F8. The exemplified therapeutic antibodies may be further labeled or combined with a radioisotope particle, such as indium In 111, yttrium Y 90, iodine I-131.

In a certain embodiments, the additional therapeutic agent is a nitrogen mustard alkylating agent. Nonlimiting examples of nitrogen mustard alkylating agents include chlorambucil.

In one embodiment, the one or more additional therapeutic agent may be an inhibitor to Abl, activated CDC kinase (ACK), adenosine A2B receptor (A2B), apoptosis signal-regulating kinase (ASK) such as ASK1, Auroa kinase, BTK, BRD such as BRD4, c-Kit, c-Met, CDK-activating kinase (CAK), calmodulin-dependent protein kinase (CaMK), cyclin-dependent kinase (CDK), casein kinase (CK), discoidin domain receptor (DDR) such as DDR1 and/or DDR2, EGFR, focal adhesion kinase (FAK), Flt-3, FYN, glycogen synthase kinase (GSK), HCK, histone deacetylase (HDAC), IKK such as IKKβε, isocitrate dehydrogenase (IDH) such as IDH1, IKK, JAK such as JAK1, JAK2 and/or JAK3, KDR, lymphocyte-specific protein tyrosine kinase (LCK), lysyl oxidase protein, lysyl oxidase-like protein (LOXL) such as LOXL1, LOXL2, LOXL3, LOXL4, and/or LOXL5, LYN, matrix metalloprotease (MMP) such as MMP 1-10, MEK, mitogen-activated protein kinase (MAPK), NEK9, NPM-ALK, p38 kinase, platelet-derived growth factor (PDGF), phosphorylase kinase (PK), polo-like kinase (PLK), PI3K such as PI3Kγ, PI3Kδ, PI3Kβ, PI3Kα and/or pan-PI3K, protein kinase (PK) such as protein kinase A, B, and/or C, PYK, SYK, serine/threonine kinase TPL2, serine/threonine kinase STK, signal transduction and transcription (STAT), SRC, serine/threonine-protein kinase (TB K) such as TBK1, TIE, tyrosine kinase (TK), VEGFR, YES, or any combination thereof. In certain embodiment, the one or more therapeutic agents are a PI3K inhibitor and a JAK inhibitor such as PI3Kγ, PI3Kδ, PI3Kβ, PI3Kα and/or pan-PI3K, such as JAK1, JAK2 and/or JAK3. In another embodiment, the one or more therapeutic agents are a PI3Kα inhibitor and a JAK inhibitor.

By way of example, the one or more therapeutic agent is: a JAK inhibitor, including but not limited to Compound A, ruxolitinib, fedratinib, tofacitinib, baricitinib, lestaurtinib, pacritinib, XL019, AZD1480, INCB039110, LY2784544, BMS911543, and NS018; a myelofibrosis inhibiting agent, including but not limited to, hedgehog inhibitors (saridegib), histone deacetylase (HDAC) inhibitors (pracinostat, panobinostat), tyrosine kinase inhibitor (lestaurtinib); a discoidin domain receptor (DDR) inhibitor, including but not limited to, those disclosed in US2009/0142345, US2011/0287011, WO2013/027802, WO2013/034933, and U.S. Provisional Application No. 61/705,044; a MMP9 inhibitor, including but not limited to, marimastat (BB-2516), cipemastat (Ro 32-3555), and those described in WO2012/027721; a LOXL inhibitor, including but not limited to the antibodies described in WO2009/017833; a LOXL2 inhibitor, including but not limited to the antibodies described in WO2009/017833, WO2009/035791 and WO/2011/097513; an ASK1 inhibitor, including but not limited to, those described in WO2011/008709 and WO/2013/112741; a PI3Kδ inhibitor, including but not limited to, Compound B, Compound C, Compound D, Compound E, the compounds described in U.S. Pat. No. 7,932,260, U.S. Provisional Application Nos. 61/543,176; 61/581,528; 61/745,429; 61/745,437; and 61/835,333, PI3K II, TGR-1202, AMG-319, GSK2269557, X-339, X-414, RP5090, KAR4141, XL499, OXY111A, IPI-145, IPI-443; a PI3Kβ inhibitor, including but not limited to, GSK2636771, BAY 10824391, TGX221; a PI3Kα inhibitor, including but not limited to, Buparlisib, BAY 80-6946, BYL719, PX-866, RG7604, MLN1117, WX-037, AEZS-129, PA799; a PI3Kγ inhibitor, including but not limited to, ZSTK474, AS252424, LY294002, TG100115; a pan PI3K inhibitor, including but not limited to, LY294002, BEZ235, XL147 (SAR245408), GDC-0941; additional PI3K inhibitors, including but not limited to BKM120, CH5132799, XL756, and GDC-0980, wortmannin; a BTK inhibitor, including but not limited to, ibrutinib, HM71224, ONO-4059, CC-292; a SYK inhibitor, including but not limited to, tamatinib (R406), fostamatinib (R788), PRT062607, BAY-61-3606, NVP-QAB 205 AA, R112, R343, or those described in U.S. Pat. No. 8,450,321; a BRD4 inhibitor; a tyrosine-kinase inhibitor (TKI) including but not limited to gefitinib and erlotinib (those target epidermal growth factor receptor or EGFR) and sunitinib (that targets receptors for FGF, PDGF and VEGF); an IDH1 inhibitor; a TPL2 inhibitor; an A2B inhibitor; a TBK1 inhibitor; a IKK inhibitor; or agents that activate or reactivate latent human immunodeficiency virus (HIV) including but not limited to panobinostat; a protein kinase C (PKC) activator; and romidepsin.

In other aspects, the combination of a PI3Kδ inhibitor (e.g., Compound B, Compound C, or Compound C and Compound B together) and an anti-CD20 antibody (e.g., obinutuzumab) as described herein is used in combination with one or more additional therapeutic agents that are being used and/or developed to treat cancers or inflammatory disorders. The one or more additional therapeutic agents may be an inhibitor to PI3K such as PI3Kγ, PI3Kβ, and/or PI3Kα, Janus kinase (JAK) such as JAK1, JAK2 and/or JAK3, spleen tyrosine kinase (SYK), or Bruton's tyrosine kinase (BTK); a bromodomain containing protein inhibitor (BRD) such as BRD4, a lysyl oxidase protein (LOX), lysyl oxidase-like protein (LOXL) such as LOXL1-5, a matrix metalloprotease (MMP) such as MMP 1-10, an adenosine A2B receptor (A2B), an isocitrate dehydrogenase (IDH) such as IDH1, apoptosis signal-regulating kinase (ASK) such as ASK1, serine/threonine kinase TPL2, discoidin domain receptor (DDR) such as DDR1 and DDR2, histone deacetylase (HDAC), protein kinase C (PKC), or any combination thereof. In certain other embodiments, the one or more additional therapeutic agents include, without limitation, anti-PD-1 antibodies (e.g., nivolimumab (BMS-936558 or MDX1106) or MK-34775) and anti-PD-L1 antibodies (e.g., BMS-936559. MPDL3280A, MEDI4736, MSB0010718C, and MDX1105-01_.

One, two, three, or more of the additional therapeutic agents (e.g. a PI3K inhibitor, a JAK inhibitor, a SYK inhibitor, a BTK inhibitor, a BRD4 inhibitor, a LOXL2 inhibitor, a MMP9 inhibitor, an A2B inhibitor, an IDH inhibitor, an ASK inhibitor, a TPL2 inhibitor, a DDR1 inhibitor, a TBK inhibitor, a HDAC inhibitor, a PKC inhibitor) may be further used or combined with a chemotherapeutic agent, an immunotherapeutic agent, a radiotherapeutic agent, an anti-neoplastic agent, an anti-cancer agent, an anti-fibrotic agent, an anti-angiogenic agent, a therapeutic antibody, or any combination thereof.

Exemplary PI3K inhibitors include, without limitation duvelisib (IPI-145).

In some embodiment, the methods, compositions, kits, and articles of manufacture for treating hyperproliferative disorders, such as cancers and MPN, use or include a PI3Kδ inhibitor and/or a JAK1/2 inhibitor. One, two, three, or more of the inhibitors or therapeutic agents may be further used or combined with a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, a therapeutic antibody, or any combination thereof.

In certain embodiments, the methods, compositions, kits, and articles of manufacture for treating MPN that use or include Compound A or a pharmaceutically acceptable salt thereof or ruxolitinib or a pharmaceutically acceptable salt thereof as the JAK inhibitor; and Compound B or a pharmaceutically acceptable salt thereof, Compound C or a pharmaceutically acceptable salt thereof, Compound D or a pharmaceutically acceptable salt thereof, or Compound E or a pharmaceutically acceptable salt thereof as the PI3Kδ inhibitor. In other embodiments, the JAK inhibitor is Compound A or a pharmaceutically acceptable salt thereof. In another embodiment, the JAK inhibitor is ruxolitinib or a pharmaceutically acceptable salt thereof. In additional embodiments, the PI3K inhibitor is Compound B or a pharmaceutically acceptable salt thereof. In other embodiments, the PI3K inhibitor is Compound C or a pharmaceutically acceptable salt thereof. In some other embodiments, the PI3K inhibitor is Compound D or a pharmaceutically acceptable salt thereof. In yet another embodiment, the PI3K compound is Compound E or a pharmaceutically acceptable salt thereof.

In other embodiment, the methods, compositions, kits, and articles of manufacture for treating cancers use or include a PI3Kδ inhibitor and/or an anti-CD20 antibody. In certain embodiments, the methods, compositions, kits, and articles of manufacture for treating cancers use or include Compound B or Compound C, or a pharmaceutically acceptable salt thereof, as the PI3Kδ inhibitor. In certain embodiments, the methods, compositions, kits, and articles of manufacture for treating cancers use or include obinutuzumab as the anti-CD20 antibody.

### Methods for Treatment

The present application provides methods for treating hyperproliferative diseases in a subject (e.g., a human) comprising administering to the subject (e.g., a human) a therapeutically effective amount of one or more of inhibitors, including a PI3K inhibitor, a JAK inhibitor, a SYK inhibitor, a BTK inhibitor, and/or a BRD inhibitor. In one embodiment, the method comprises administering to the subject (i.e. a human) a therapeutically effective amount of a JAK inhibitor, including a JAK1/2 inhibitor. In another embodiment, the method comprises administering to the subject (i.e. a human) a therapeutically effective amount of a PI3K inhibitor, including a PI3Kδ inhibitor. In additional embodiment, the method comprises administering to the subject (i.e. a human) a therapeutically effective amount of a JAK inhibitor, a therapeutically effective amount of a PI3K inhibitor, and a therapeutically effective amount of additional therapeutic agent. In certain embodiments, the method comprises a therapeutically effective amount of a JAK inhibitor and a therapeutically effectively amount of a PI3Kδ inhibitor. In some embodiments, the method comprises administering to a human a therapeutically effective amount of Compound A or ruxolotinib, or a pharmaceutically acceptable salt thereof, and a therapeutically effective amount of Compound B, Compound C, Compound D, or Compound E, or a pharmaceutically acceptable salt thereof. In one embodiment, the method comprises administering to a human a therapeutically effective amount of Compound A or a pharmaceutically acceptable salt thereof, and a therapeutically effective amount of Compound B, C, D, or E. In another embodiment, the method comprises administering to a human a therapeutically effective amount of Compound A or a pharmaceutically acceptable salt thereof, and a therapeutically effective amount of Compound B or a pharmaceutically acceptable salt thereof. In other embodiment, the method comprises administering to a human a therapeutically effective amount of ruxolitinib or a pharmaceutically acceptable salt thereof, and a therapeutically effective amount of Compound B, C, D, or E. In yet another embodiment, the method comprises administering to a human a therapeutically effective amount of ruxolotinib or a pharmaceutically acceptable salt thereof, and a therapeutically effective amount of Compound B or a pharmaceutically acceptable salt thereof.

The present disclosure also provides methods for treating cancer in a subject (e.g., a human) comprising administering to the subject (e.g., a human) a therapeutically effective amount of a PI3Kδ inhibitor and a therapeutically effective amount of an anti-CD20 antibody. In some embodiments, the method comprises administering to the subject (e.g., a human) a therapeutically effective amount of Compound B or Compound C, or a pharmaceutically acceptable salt thereof, and a therapeutically effective amount of an anti-CD20 antibody. In one embodiment, the method comprises administering to the subject (e.g., a human) a therapeutically effective amount of Compound B or Compound C, or a pharmaceutically acceptable salt thereof, and a therapeutically effective amount of obinutuzumab. In other embodiment, the method comprises administering to a human in need thereof a therapeutically effective amount of Compound B or Compound C, or a pharmaceutically acceptable salt thereof and a therapeutically effective amount of obinutuzumab; and the human having or is suspect of having a cancer.

The subject may be a human, who is a patient. The patients may have or have not received prior drug therapy. In one embodiment, the method provides a treatment or therapeutic to hyperproliferative disease patients who have been treated or are currently being treated with thalidomide or with a derivative thereof, such as lenalidomide, or other JAK inhibitor such as ruxolotinib or TG101348. In certain embodiments, the method comprises treating patients who have received prior drug treatment using a JAK inhibitor.

In some embodiments, the method comprises treating patients who have received prior drug treatment using a JAK inhibitor over a period of time (i.e. chronic JAK therapy) and developed disease persistence. Patients who have received chronic ruxolitinib (i.e. over 3-6 months, more than 6 months, or more than one year) commonly develop disease persistence. As used herein, disease persistence refers to patients showing gradual return of splenomegaly and/or constitutional symptoms, the lack of hematologic or molecular remissions, or the loss of clinical improvement.

The hyperproliferative disease includes cancer and myeloproliferative disease such as cellular-proliferative disease in cardiac, lung, gastrointestine, genitourinary tract, liver, bone, nerve system, gynecological, hematological, skin, and adrenal glands.

In certain embodiments, a method for treating cancer is provided, wherein the method comprises administering to a patient in need thereof a therapeutically effective amount of a PI3Kδ inhibitor and a therapeutically effective amount of an anti-CD20 antibody, wherein the patient has not been previously treated.

In certain embodiments, a method for treating leukemia is provided, wherein the method comprises administering to a patient in need thereof a therapeutically effective amount of a PI3Kδ inhibitor and a therapeutically effective amount of an anti-CD20 antibody, wherein the patient has not been previously treated.

In certain embodiments, a method for treating chronic lyphocytic leukemia is provided, wherein the method comprises administering to a patient in need thereof a therapeutically effective amount of a PI3Kδ inhibitor and a therapeutically effective amount of an anti-CD20 antibody, wherein the patient has not been previously treated.

In certain embodiments, a method for treating lymphoma is provided, wherein the method comprises administering to a patient in need thereof a therapeutically effective amount of a PI3Kδ inhibitor and a therapeutically effective amount of an anti-CD20 antibody, wherein the patient has not been previously treated. In certain embodiments, the lymphoma is non-Hodgkin lymphoma (NHL). In certain embodiments, the lymphoma is indolent non-Hodgkin lymphoma (iNHL). In certain embodiments, the lymphoma is Follicular B-cell non-Hodkin lymphoma (FL) or small lymphocytic lymphoma (SLL).

In certain embodiments, a method for treating cancer is provided, wherein the method comprises administering to a patient in need thereof a therapeutically effective amount of a PI3Kδ inhibitor and a therapeutically effective amount of an anti-CD20 antibody, wherein the patient is not eligible for treatment with bendamustine and rituximab.

In certain embodiments, a method for treating leukemia is provided, wherein the method comprises administering to a patient in need thereof a therapeutically effective amount of a PI3Kδ inhibitor and a therapeutically effective amount of an anti-CD20 antibody, wherein the patient is not eligible for treatment with bendamustine and rituximab.

In certain embodiments, a method for treating chronic lyphocytic leukemia is provided, wherein the method comprises administering to a patient in need thereof a therapeutically effective amount of a PI3Kδ inhibitor and a therapeutically effective amount of an anti-CD20 antibody, wherein the patient is not eligible for treatment with bendamustine and rituximab.

In certain embodiments, a method for treating lymphoma is provided, wherein the method comprises administering to a patient in need thereof a therapeutically effective amount of a PI3Kδ inhibitor and a therapeutically effective amount of an anti-CD20 antibody, wherein the patient is not eligible for treatment with bendamustine and rituximab. In certain embodiments, the lymphoma is indolent non-Hodgkin lymphoma (iNHL). In certain embodiments, the lymphoma is Follicular B-cell non-Hodkin lymphoma (FL) or small lymphocytic lymphoma (SLL).

In certain embodiments, a method for treating cancer is provided, wherein the method comprises administering to a patient in need thereof a therapeutically effective amount of a PI3Kδ inhibitor and a therapeutically effective amount of an anti-CD20 antibody, wherein the patient is not eligible for treatment with fludarabine, cyclophosphamide and rituximab.

In certain embodiments, a method for treating leukemia is provided, wherein the method comprises administering to a patient in need thereof a therapeutically effective amount of a PI3Kδ inhibitor and a therapeutically effective amount of an anti-CD20 antibody, wherein the patient is not eligible for treatment with fludarabine, cyclophosphamide and rituximab.

In certain embodiments, a method for treating chronic lyphocytic leukemia is provided, wherein the method comprises administering to a patient in need thereof a therapeutically effective amount of a PI3Kδ inhibitor and a therapeutically effective amount of an anti-CD20 antibody, wherein the patient is not eligible for treatment with fludarabine, cyclophosphamide and rituximab.

In certain embodiments, a method for treating lymphoma is provided, wherein the method comprises administering to a patient in need thereof a therapeutically effective amount of a PI3Kδ inhibitor and a therapeutically effective amount of an anti-CD20 antibody, wherein the patient is not eligible for treatment with fludarabine, cyclophosphamide and rituximab. In certain embodiments, the lymphoma is indolent non-Hodgkin lymphoma (iNHL). In certain embodiments, the lymphoma is Follicular B-cell non-Hodkin lymphoma (FL) or small lymphocytic lymphoma (SLL).

Myeloproliferative Disease

Myeloproliferative diseases (MPD) or myeloproliferative neoplasms (MPN) are a diverse group of clonal disorders of pluripotent hematopoietic stem cells that have increase or overproduction of one or more myeloid cells, growth factor independent colony formation in vitro, marrow hypercellularity, extramedullary hematopoiesis, splenomegaly, hepatomegaly, and thrombotic and/or hemorrhagic diathesis. The myleoproliferative diseases or neoplasms include, but are not limited to, polycythemia vera, primary myelofibrosis, thrombocythemia, essential thrombocythemia, agnoneic myeloid metaplasia, idiopathic myelofibrosis, chronic myelogenous leukemia, systemic mastocystosis, chronic neutrophilic leukemia, myelodisplastic syndrome, and systemic mast cell disease. In some embodiments, the myloproliferative disease is polycythemia vera, essential thrombocythemia, and primary myelofibrosis. In certain embodiments, the myloproliferative disease is polycythemia vera. In other embodiment, the myeloproliferative disease is essential thrombocythemia. In another embodiment, the myeloproliferative disease is primary myelofibrosis.

The chronic myeloproliferative neoplasms (MPNs) are acquired marrow disorders characterized by excessive production of mature myeloid cells. Major morbidity from these conditions result from thrombo-hemorrhagic complications (arterial and venous thrombosis, major bleeding) and transformation to acute leukemia such as acute myeloid leukemia (AML). Myelofibrosis originates from acquired mutations that alter the hematopoietic stem cell and produce alterations in the kinase-mediated signaling processes, resulting in clonal myeloproliferation, bone marrow fibrosis, and abnormal cytokine expression (Tefferi et al., Blood 108:1497-503, 2006). PMF is a rare disease with an incidence of 0.4 to 1.3 per 100,000 people in Europe, Australia, and U.S. Myelofibrosis can also occur in patients with PV (10-20% of subjects after 10-20 years) and ET (2-3% of subjects), in which case it is called post-ET/PV MF. The pathogenic mechanism in PMF may be the unchecked proliferation of a hematopoietic stem cell clone that leads to ineffective erythropoiesis, atypical megakaryocytic hyperplasia, and an increase in the ratio of immature granulocytes to total granulocytes. The clonal myeloproliferation is characteristically accompanied by bone marrow fibrosis and extramedullary hematopoiesis in the spleen, liver, and other organs. Other features of extramedullary hematopoiesis on a blood smear include teardrop-shaped red cells, nucleated red cells, and myeloid immaturity. Additional clinical features include marked splenomegaly, progressive anemia, and constitutional symptoms.

An international working group (IWG) for myeloproliferative neoplasms research and treatment (IWG-MRT) has defined myeloproliferative diseases and related conditions (Vannucchi et al., CA Cancer J. Clin. 59:171-191, 2009) that are used in the present application. Patients, who present with MPN or PMF, are identifiable in the art using the IWG-MRT criteria. Subjects “at risk for” certain MPN are subjects having an early stage form of the disease, and may for instance include subjects having a genetic marker thereof, such as the JAK2V617F allele which is associated with PV (>95%), with ET (60%) and with PMF (60%). In addition, subjects are considered to be “at risk for” certain MPN if they already manifest symptoms of an earlier stage form. For example, subjects presenting with MPN are at risk for post-PV and post-ET, both of which develop following MPN.

Compound A is a JAK inhibitor and provides improved clinical response in MPN patients, including PMF. One of the improved outcomes is improvement in anemia response and/or in spleen response. By “anemia response” is meant an increase in the patient's hemoglobin level or a patient who was transfusion dependent becoming transfusion independent. Desirably, a minimum increase in hemoglobin of 2.0 g/dL lasting a minimum of 8 weeks is achieved, which is the level of improvement specified in the International Working Group (IWG) consensus criteria. However, smaller, but still medically significant, increases in hemoglobin are also considered to be within the term “anemia response”. By “spleen response” is meant a reduction in the size of the patient's spleen as assessed by either palpation of a previously palpable spleen during physical exam or by diagnostic imaging. The IWG consensus criteria specifies that there be either a minimum 50% reduction in palpable splenomegaly (spleen enlargement) of a spleen that is at least 10 cm at baseline (prior to treatment) or of a spleen that is palpable at more than 5 cm at baseline becomes not palpable. However, smaller reductions are also considered to be within the term “spleen response”.

One aspect of the present application provides the methods, composition, and kit for the patient who has received prior drug therapy or is current in drug therapy. By way of example, the patients have been treated, or are currently being treated, with thalidomide, lenalidomide, pomalidomide or derivative thereof, that are used in the treatment of multiple myeloma, and appear also to be showing some benefit in patients afflicted with myeloproliferative disorder. In another example, the patients have been treated, or are undergoing treatment, with a JAK inhibitor other than Compound A, including but not limited to INCB018424, TG101348, ruxolitinib. Patients will either be undergoing treatment with the other JAK2 inhibitor or will have been treated with such a drug within a time frame, relative to the composition or treatment provided herein, sufficient for the effects of that JAK2 inhibitor to be manifest in the patient. In general, INCB018424 is administered at starting doses of 15 or 20 mg BID with dose titration from 5 mg BID to 25 mg BID; TG101348 is administered once a day with a maximum tolerated dose (MTD) determined to be 680 mg/day; and ruxolitinib is administered at a stable dose of 20, 15, or 5 mg (based on platelet count) BID.

In certain embodiment, the MPD patients have not received any drug treatment, i.e. naïve. The naïve MPD patients may subsequently receive treatment or therapeutic described herein. For example, the naïve MPD patients may receive a PI3K inhibitor, a JAK inhibitor, additional therapeutic agent, or any combination thereof.

Patients receive the treatment or composition according to the present application experience an improved response when they are selected initially based on an elevation in the level of any one or more of the markers noted above. An elevated level is a level that is greater than the level in a normal subject. As used herein, the “level” of a given marker is considered to be altered, i.e., either elevated or reduced, when the level measured in a given patient is different to a statistically significant extent from the corresponding level in a normal subject. Patients that present with marker levels altered to an extent sufficient, desirably, to yield a p value of at least 0.05 or more significant, i.e., better, are suitable candidate for the therapy described herein. In embodiments, the p value is at least 0.03, 0.02 or 0.01, and in preferred embodiments the p value is at least 0.009, 0.007, 0.005, 0.003, 0.001 or better. The levels of a given marker can be determined using assays already well established for detection the markers noted above. In embodiments, this is achieved by extracting a biological sample from the patient candidate, such as a sample of whole blood or a fraction thereof such as plasma or serum. The sample then is treated to enrich for the marker of interest, if desired, and the enriched or neat sample is assayed for instance using a detectable ligand for the marker, such as a labeled antibody that binds selectively to the marker. The amount of marker present in the sample can then be determined either semi-quantitatively or quantitatively, to obtain a value that is then compared against a reference value that is the normal level for that marker in a healthy subject. As noted above, a difference in marker levels sufficient to arrive at a p value that is at least 0.05 indicates an altered marker level of significance, and patients presenting with an elevated level of that marker (or in the case of eotaxin, a decreased level) are candidates to be treated using the method, composition, kit of the present application.

Also suitable as candidates for the therapy are those patients that meet certain clinical criteria, including those presenting with a spleen of relatively small size, and those presenting with an elevated level of circulating, or peripheral, blasts. In one embodiment, the selected patient is one that has not yet progressed to transfusion dependency. Splenic enlargement is assessed by palpation. Splenic size and volume can also be measured by diagnostic imaging such as ultrasound, CT or MRI). Normal spleen size is approximately 11.0 cm. in craniocaudal length.

Also suitable as candidates for the therapy are those patients presenting with a lower percentage of circulating blasts. Blasts are immature precursor cells that are normally found in the bone marrow and not the peripheral blood. They normally give rise to mature blood cells. The lower percentage of circulating blasts is measured by cytomorphologic analysis of a peripheral blood smear as well as multiparameter flow cytometry and immunohistochemistry. As a prognostic factor >/=1% blasts is used.

In another aspect, the application provides the methods, composition, and kits for the patients who have received prior therapy and exhibit suboptimal response. The suboptimal response to prior drug therapy may be characterized by ineffective erythropoiesis and bone marrow fibrosis with extramedullary hematopoiesis manifested by marked hepatosplenomegaly due in part to the emergence of a clone of cells that are non-responsive or resistant to the prior drug therapy. It has been shown that patients receive ruxolitinib develop resistance or non-response after a period of time. Such disease may be observed after 3, 4, 5, 6, 7, 8, 9, 10, 11, or 12 months or years of ruxolitinb treatment.

The biologic mechanism for suboptimal responses is unclear. Although resistance mutations within JAK2 have not been identified as a basis for acquired resistance to JAK inhibitors, heterodimeric JAK-STAT activation is a potential mechanism of disease persistence. JAK inhibitor persistent cells may develop through exposure to JAK inhibitors, and such cells may exhibit lower apoptosis in response to ongoing exposure these drugs. This may cause reactivation of JAK2 phosphorylation and the downstream STAT3, STATS, and MAP kinase signaling in persistent cells which would no longer be inhibited by JAK inhibitors. It is suggested that JAK family members JAK1 and TYK2 associate with JAK2 in persistent cells, resulting in re-activation of JAK2.

The persistence phenomenon is reversible, and cells become re-sensitized or responsive with withdrawal of the JAK inhibitor. These re-sensitized cells suggest a loss of the association between JAK1/TYK2 and JAK2, resulting in loss of JAK2 activation. This phenomenon of JAK inhibitor persistence is observed in vivo in MPN murine models, and in primary samples of patients treated with the ruxolitinib.

The present application shows that the PI3Kδ isoform was expressed and the prominent isoform (i.e. highest expression levels) among PI3K isoforms α, β, δ, and γ in progenitor cells from MF patients. In addition, the present application showed that PI3Kδ inhibitors inhibited thrombopoietin (TPO)-treated and basal (TPO-untreated) AKT/S6RP phosphorylation (p-AKT/p-S6RP) in PBMC from MF patients. MF patients were either on chronic ruxolitinib therapy or had not received ruxolitinib or other JAK inhibitors (i.e. naïve). It is hypothesized that, upon activation of the MPL receptor by thrombopoietin (TPO), JAK2 is recruited to the membrane which activates downstream signaling pathways including STATS/5, PI3K and RAS, resulting in increased proliferation, survival, metabolism and cellular motility. About 50-60% of primary MF patients harbor the activating JAK2V617F mutation which constitutively activates the signaling cascade.

According to the present application, the combination of a PI3Kδ inhibitor and a JAK inhibitor results in enhanced therapeutic responses (including beneficial or synergistic effects). Also, concurrent targeting of PI3K and JAK/STAT pathway may represent a new therapeutic treatment to optimize efficacy and reduce toxicity in patients with MPN.

Cancers

The methods described herein may be used to treat various types of cancers. In some embodiments, the cancer may be a hematological malignancy, including relapsed or refractory hematologic malignancies. Cancers amenable to treatment using the methods described herein may include leukemias, lymphomas, and multiple myeloma. Leukemias may include, for example, lymphocytic leukemias and chronic myeloid (myelogenous) leukemias. Lymphomas may include, for example, malignant neoplasms of lymphoid and reticuloendothelial tissues, such as Burkitt's lymphoma, Hodgkin's lymphoma, non-Hodgkin's lymphomas (including, for example, indolent non-Hodgkin's lymphoma), and lymphocytic lymphomas.

In some embodiments, the cancer is Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), B-cell ALL, acute myeloid leukemia (AML), chronic lymphocytic leukemia (CLL), small lymphocytic lymphoma (SLL), myelodysplastic syndrome (MDS), myeloproliferative disease (MPD), mantle cell lymphoma (MCL), follicular lymphoma (FL), Waldestrom's macroglobulinemia (WM), T-cell lymphoma, B-cell lymphoma, diffuse large B-cell lymphoma (DLBCL), or marginal zone lymphoma (MZL). In one embodiment, the cancer is minimal residual disease (MRD). In additional embodiment, the cancer is selected from Hodgkin's lymphoma, non-Hodgkin's lymphoma (NHL), indolent non-Hodgkin's lymphoma (iNHL), and refractory iNHL. In certain embodiment, the cancer is indolent non-Hodgkin's lymphoma (iNHL). In some embodiment, the cancer is refractory iNHL. In one embodiment, the cancer is chronic lymphocytic leukemia (CLL). In other embodiment, the cancer is diffuse large B-cell lymphoma (DLBCL).

In one embodiment, the cancer is relapsed chronic lymphocytic leukemia (CLL). In one embodiment, the cancer is follicular B-cell non-Hodgkin lymphoma. In one embodiment, the cancer is relapsed follicular B-cell non-Hodgkin lymphoma. In one embodiment, the cancer is small lymphocytic lymphoma. In one embodiment, the cancer is relapsed small lymphocytic lymphoma.

In certain embodiments, the cancer is acute lymphocytic leukemia (ALL), B-cell ALL, acute myeloid leukemia (AML), chronic lymphocytic leukemia (CLL), small lymphocytic lymphoma (SLL), follicular lymphoma, multiple myeloma (MM), non-Hodgkin's lymphoma (NHL), indolent NHL (iNHL), mantle cell lymphoma (MCL), follicular lymphoma, Waldenstrom's macroglobulinemia (WM), B-cell lymphoma, or diffuse large B-cell lymphoma (DLBCL).

In some embodiments, provided are methods of treating cancer in a subject (e.g., a human) by administering to the subject a therapeutically effective amount of Compound B or Compound C, or a pharmaceutically acceptable salt thereof, and a therapeutically effective amount of obinutuzumab, wherein the cancer is leukemia. In some embodiments, the leukemia is chronic leukemia. An example of chronic leukemia is chronic lymphocytic leukemia (CLL). In one embodiment, the leukemia is minimal residual disease (MRD).

In other embodiments, provided are also methods of treating cancer in a subject by administering to the subject (e.g. a human) a therapeutically effective amount of Compound B or Compound C, or a pharmaceutically acceptable salt thereof, and a therapeutically effective amount of obinutuzumab, wherein the cancer is lymphoma. In some embodiments, the lymphoma is non-Hodgkin's lymphoma (NHL). An example of non-Hodgkin's lymphoma is indolent NHL (iNHL), or refractory iNHL. In some embodiments, the lymphoma is follicular lymphoma or small lymphocytic lymphoma.

In certain embodiments, the cancer is a solid tumor is selected from the group consisting of pancreatic cancer; bladder cancer; colorectal cancer; breast cancer, including metastatic breast cancer; prostate cancer, including androgen-dependent and androgen-independent prostate cancer; renal cancer, including, e.g., metastatic renal cell carcinoma; hepatocellular cancer; lung cancer, including, e.g., non-small cell lung cancer (NSCLC), bronchioloalveolar carcinoma (BAC), and adenocarcinoma of the lung; ovarian cancer, including, e.g., progressive epithelial or primary peritoneal cancer; cervical cancer; gastric cancer; esophageal cancer; head and neck cancer, including, e.g., squamous cell carcinoma of the head and neck; melanoma; neuroendocrine cancer, including metastatic neuroendocrine tumors; brain tumors, including, e.g., glioma, anaplastic oligodendroglioma, adult glioblastoma multiforme, and adult anaplastic astrocytoma; bone cancer; and soft tissue sarcoma. In certain embodiments, the cancer is pancreatic cancer.

Any of the methods of treatment provided may be used to treat cancer at various stages. By way of example, the cancer stage includes but is not limited to early, advanced, locally advanced, remission, refractory, reoccurred after remission and progressive.

Subjects

Any of the methods of treatment provided may be used to treat a subject (e.g., human) who has been diagnosed with or is suspected of having cancer. As used herein, a subject refers to a mammal, including, for example, a human.

In some embodiments, the subject may be a human who exhibits one or more symptoms associated with cancer or hyperproliferative disease. In some embodiments, the subject may be a human who exhibits one or more symptoms associated with cancer. In some embodiments, the subject is at an early stage of a cancer. In other embodiments, the subject is at an advanced stage of cancer.

In certain, the subject may be a human who is at risk, or genetically or otherwise predisposed (e.g., risk factor) to developing cancer or hyperproliferative disease who has or has not been diagnosed. As used herein, an “at risk” subject is a subject who is at risk of developing cancer. The subject may or may not have detectable disease, and may or may not have displayed detectable disease prior to the treatment methods described herein. An at risk subject may have one or more so-called risk factors, which are measurable parameters that correlate with development of cancer, which are described herein. A subject having one or more of these risk factors has a higher probability of developing cancer than an individual without these risk factor(s). These risk factors may include, for example, age, sex, race, diet, history of previous disease, presence of precursor disease, genetic (e.g., hereditary) considerations, and environmental exposure. In some embodiments, the subjects at risk for cancer include, for example, those having relatives who have experienced the disease, and those whose risk is determined by analysis of genetic or biochemical markers.

In addition, the subject may be a human who is undergoing one or more standard therapies, such as chemotherapy, radiotherapy, immunotherapy, surgery, or combination thereof. Accordingly, one or more kinase inhibitors may be administered before, during, or after administration of chemotherapy, radiotherapy, immunotherapy, surgery or combination thereof.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

In certain embodiments, the subject is refractory to at least one, at least two, at least three, or at least four chemotherapy treatment (including standard or experimental chemotherapy) selected from fludarabine, rituximab, obinutuzumab, alkylating agents, alemtuzumab and other chemotherapy treatments such as CHOP (cyclophosphamide, doxorubicin, vincristine, prednisone); R-CHOP (rituximab-CHOP); hyperCVAD (hyperfractionated cyclophosphamide, vincristine, doxorubicin, dexamethasone, methotrexate, cytarabine); R-hyperCVAD (rituximab-hyperCVAD); FCM (fludarabine, cyclophosphamide, mitoxantrone); R-FCM (rituximab, fludarabine, cyclophosphamide, mitoxantrone); bortezomib and rituximab; temsirolimus and rituximab; temsirolimus and Velcade®; Iodine-131 tositumomab (Bexxar®) and CHOP; CVP (cyclophosphamide, vincristine, prednisone); R-CVP (rituximab-CVP); ICE (iphosphamide, carboplatin, etoposide); R-ICE (rituximab-ICE); FCR (fludarabine, cyclophosphamide, rituximab); FR (fludarabine, rituximab); and D.T. PACE (dexamethasone, thalidomide, cisplatin, Adriamycin®, cyclophosphamide, etoposide). In some embodiments, the subject is refractory to rituximab.

Other examples of chemotherapy treatments (including standard or experimental chemotherapies) are described below. In addition, treatment of certain lymphomas is reviewed in Cheson, B. D., Leonard, J. P., “Monoclonal Antibody Therapy for B-Cell Non-Hodgkin's Lymphoma” The New England Journal of Medicine 2008, 359(6), p. 613-626; and Wierda, W. G., “Current and Investigational Therapies for Patients with CLL” Hematology 2006, p. 285-294. Lymphoma incidence patterns in the United States is profiled in Morton, L. M., et al. “Lymphoma Incidence Patterns by WHO Subtype in the United States, 1992-2001” Blood 2006, 107(1), p. 265-276.

Examples of immunotherapeutic agents treating lymphoma or leukemia include, but are not limited to, rituximab (such as Rituxan), alemtuzumab (such as Campath, MabCampath), anti-CD19 antibodies, anti-CD20 antibodies, anti-MN-14 antibodies, anti-TRAIL, Anti-TRAIL DR4 and DR5 antibodies, anti-CD74 antibodies, apolizumab, bevacizumab, CHIR-12.12, epratuzumab (hLL2-anti-CD22 humanized antibody), galiximab, ha20, ibritumomab tiuxetan, lumiliximab, milatuzumab, obinutuzumab, ofatumumab, PRO131921, SGN-40, WT-1 analog peptide vaccine, WT1 126-134 peptide vaccine, tositumomab, autologous human tumor-derived HSPPC-96, and veltuzumab. Additional immunotherapy agents includes using cancer vaccines based upon the genetic makeup of an individual patient's tumor, such as lymphoma vaccine example is GTOP-99 (MyVax®). In one embodiment, the immunotherapy agent is anti-CD20 antibody. In other embodiment, the immunotherapy agent is obinutuzumab. In some embodiment, the method comprising administering an therapeutically effective amount of Compound B and an therapeutically effective amount of obinutuzumab to a patient in need thereof. The administration of Compound B may be prior, concurrently, or subsequent to the administration of obinutuzumab. As shown in the present application, the combination of Compound B and obinutuzumab may provide desired therapeutic benefits compared to obinutuzumab alone or combined with other agents. One benefit may be the increased cell death of cancerous cells by the combination of Compound B and obinutuzumab, compared to those of obinutuzumab alone. Other benefit may be the desired safety profile of the combination of Compound B and obinutuzumab compared to the combination of obinutuzumab with other agents as other agents may interfere with the immune effector function and in vivo efficacy of obinutuzumab.

Examples of chemotherapy agents for treating lymphoma or leukemia include aldesleukin, alvocidib, antineoplaston AS2-1, antineoplaston A10, anti-thymocyte globulin, amifostine trihydrate, aminocamptothecin, arsenic trioxide, beta alethine, Bcl-2 family protein inhibitor ABT-263, ABT-199, ABT-737, BMS-345541, bortezomib (Velcade®), bryostatin 1, busulfan, carboplatin, campath-1H, CC-5103, carmustine, caspofungin acetate, clofarabine, cisplatin, Cladribine (Leustarin), Chlorambucil (Leukeran), Curcumin, cyclosporine, Cyclophosphamide (Cyloxan, Endoxan, Endoxana, Cyclostin), cytarabine, denileukin diftitox, dexamethasone, DT PACE, docetaxel, dolastatin 10, Doxorubicin (Adriamycin®, Adriblastine), doxorubicin hydrochloride, enzastaurin, epoetin alfa, etoposide, Everolimus (RAD001), fenretinide, filgrastim, melphalan, mesna, Flavopiridol, Fludarabine (Fludara), Geldanamycin (17-AAG), ifosfamide, irinotecan hydrochloride, ixabepilone, Lenalidomide (Revlimid®, CC-5013), lymphokine-activated killer cells, melphalan, methotrexate, mitoxantrone hydrochloride, motexafin gadolinium, mycophenolate mofetil, nelarabine, oblimersen (Genasense) Obatoclax (GX15-070), oblimersen, octreotide acetate, omega-3 fatty acids, oxaliplatin, paclitaxel, PD0332991, PEGylated liposomal doxorubicin hydrochloride, pegfilgrastim, Pentstatin (Nipent), perifosine, Prednisolone, Prednisone, R-roscovitine (Selicilib, CYC202), recombinant interferon alfa, recombinant interleukin-12, recombinant interleukin-11, recombinant flt3 ligand, recombinant human thrombopoietin, rituximab, sargramostim, sildenafil citrate, simvastatin, sirolimus, Styryl sulphones, tacrolimus, tanespimycin, Temsirolimus (CC1-779), Thalidomide, therapeutic allogeneic lymphocytes, thiotepa, tipifarnib, Velcade® (bortezomib or PS-341), Vincristine (Oncovin), vincristine sulfate, vinorelbine ditartrate, Vorinostat (SAHA), vorinostat, and FR (fludarabine, rituximab), CHOP (cyclophosphamide, doxorubicin, vincristine, prednisone), CVP (cyclophosphamide, vincristine and prednisone), FCM (fludarabine, cyclophosphamide, mitoxantrone), FCR (fludarabine, cyclophosphamide, rituximab), hyperCVAD (hyperfractionated cyclophosphamide, vincristine, doxorubicin, dexamethasone, methotrexate, cytarabine), ICE (iphosphamide, carboplatin and etoposide), MCP (mitoxantrone, chlorambucil, and prednisolone), R-CHOP (rituximab plus CHOP), R-CVP (rituximab plus CVP), R-FCM (rituximab plus FCM), R-ICE (rituximab-ICE), and R-MCP (R-MCP).

The therapeutic treatments can be supplemented or combined with any of the abovementioned therapies with stem cell transplantation or treatment. One example of modified approach is radioimmunotherapy, wherein a monoclonal antibody is combined with a radioisotope particle, such as indium In 111, yttrium Y 90, iodine I-131. Examples of combination therapies include, but are not limited to, Iodine-131 tositumomab (Bexxar®), Yttrium-90 ibritumomab tiuxetan (Zevalin®), Bexxar® with CHOP.

Other therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, pharmacological study, low-LET cobalt-60 gamma ray therapy, bleomycin, conventional surgery, radiation therapy, and nonmyeloablative allogeneic hematopoietic stem cell transplantation.

For example, treatment of non-Hodgkin's lymphomas (NHL), especially of B cell origin, include the use of monoclonal antibodies, standard chemotherapy approaches (e.g., CHOP, CVP, FCM, MCP, and the like), radioimmunotherapy, and combinations thereof, especially integration of an antibody therapy with chemotherapy. Examples of unconjugated monoclonal antibodies for Non-Hodgkin's lymphoma/B-cell cancers include rituximab, alemtuzumab, human or humanized anti-CD20 antibodies, lumiliximab, anti-TRAIL, bevacizumab, galiximab, epratuzumab, SGN-40, and anti-CD74. Examples of experimental antibody agents used in treatment of Non-Hodgkin's lymphoma/B-cell cancers include ofatumumab, ha20, PRO131921, alemtuzumab, galiximab, SGN-40, CHIR-12.12, epratuzumab, lumiliximab, apolizumab, milatuzumab, and bevacizumab. Examples of standard regimens of chemotherapy for Non-Hodgkin's lymphoma/B-cell cancers include CHOP (cyclophosphamide, doxorubicin, vincristine, prednisone), FCM (fludarabine, cyclophosphamide, mitoxantrone), CVP (cyclophosphamide, vincristine and prednisone), MCP (mitoxantrone, chlorambucil, and prednisolone), R-CHOP (rituximab plus CHOP), R-FCM (rituximab plus FCM), R-CVP (rituximab plus CVP), and R-MCP (R-MCP). Examples of radioimmunotherapy for Non-Hodgkin's lymphoma/B-cell cancers include yttrium-90-labeled ibritumomab tiuxetan, and iodine-131-labeled tositumomab.

In another example, therapeutic treatments for mantle cell lymphoma (MCL) include combination chemotherapies such as CHOP (cyclophosphamide, doxorubicin, vincristine, prednisone), hyperCVAD (hyperfractionated cyclophosphamide, vincristine, doxorubicin, dexamethasone, methotrexate, cytarabine) and FCM (fludarabine, cyclophosphamide, mitoxantrone). In addition, these regimens can be supplemented with the monoclonal antibody rituximab (Rituxan) to form combination therapies R-CHOP, hyperCVAD-R, and R-FCM. Other approaches include combining any of the abovementioned therapies with stem cell transplantation or treatment with ICE (iphosphamide, carboplatin and etoposide). Other approaches to treating mantle cell lymphoma includes immunotherapy such as using monoclonal antibodies like Rituximab (Rituxan). Rituximab can be used for treating indolent B-cell cancers, including marginal-zone lymphoma, WM, CLL and small lymphocytic lymphoma. A combination of Rituximab and chemotherapy agents is especially effective. A modified approach is radioimmunotherapy, wherein a monoclonal antibody is combined with a radioisotope particle, such as Iodine-131 tositumomab (Bexxar®) and Yttrium-90 ibritumomab tiuxetan (Zevalin®). In another example, Bexxar® is used in sequential treatment with CHOP. Another immunotherapy example includes using cancer vaccines, which is based upon the genetic makeup of an individual patient's tumor. A lymphoma vaccine example is GTOP-99) (MyVax®). Yet other approaches to treating mantle cell lymphoma includes autologous stem cell transplantation coupled with high-dose chemotherapy, or treating mantle cell lymphoma includes administering proteasome inhibitors, such as Velcade® (bortezomib or PS-341), or antiangiogenesis agents, such as thalidomide, especially in combination with Rituxan. Another treatment approach is administering drugs that lead to the degradation of Bcl-2 protein and increase cancer cell sensitivity to chemotherapy, such as oblimersen (Genasense) in combination with other chemotherapeutic agents. Another treatment approach includes administering mTOR inhibitors, which can lead to inhibition of cell growth and even cell death; a non-limiting example is Temsirolimus (CCI-779), and Temsirolimus in combination with Rituxan®, Velcade® or other chemotherapeutic agents.

Other recent therapies for MCL have been disclosed (Nature Reviews; Jares, P. 2007). Such examples include Flavopiridol, PD0332991, R-roscovitine (Selicilib, CYC202), Styryl sulphones, Obatoclax (GX15-070), TRAIL, Anti-TRAIL DR4 and DR5 antibodies, Temsirolimus (CC1-779), Everolimus (RAD001), BMS-345541, Curcumin, Vorinostat (SAHA), Thalidomide, lenalidomide (Revlimid®, CC-5013), and Geldanamycin (17-AAG).

Examples of other therapeutic agents used to treat Waldenstrom's Macroglobulinemia (WM) include perifosine, bortezomib (Velcade®), rituximab, sildenafil citrate (Viagra®), CC-5103, thalidomide, epratuzumab (hLL2-anti-CD22 humanized antibody), simvastatin, enzastaurin, campath-1H, dexamethasone, DT PACE, oblimersen, antineoplaston A10, antineoplaston AS2-1, alemtuzumab, beta alethine, cyclophosphamide, doxorubicin hydrochloride, prednisone, vincristine sulfate, fludarabine, filgrastim, melphalan, recombinant interferon alfa, carmustine, cisplatin, cyclophosphamide, cytarabine, etoposide, melphalan, dolastatin 10, indium In 111 monoclonal antibody MN-14, yttrium Y 90 humanized epratuzumab, anti-thymocyte globulin, busulfan, cyclosporine, methotrexate, mycophenolate mofetil, therapeutic allogeneic lymphocytes, Yttrium Y 90 ibritumomab tiuxetan, sirolimus, tacrolimus, carboplatin, thiotepa, paclitaxel, aldesleukin, recombinant interferon alfa, docetaxel, ifosfamide, mesna, recombinant interleukin-12, recombinant interleukin-11, Bcl-2 family protein inhibitor ABT-263, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19, monoclonal antibody CD20, omega-3 fatty acids, mitoxantrone hydrochloride, octreotide acetate, tositumomab and iodine I-131 tositumomab, motexafin gadolinium, arsenic trioxide, tipifamib, autologous human tumor-derived HSPPC-96, veltuzumab, bryostatin 1, and PEGylated liposomal doxorubicin hydrochloride, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, pharmacological study, low-LET cobalt-60 gamma ray therapy, bleomycin, conventional surgery, radiation therapy, and nonmyeloablative allogeneic hematopoietic stem cell transplantation.

Examples of other therapeutic agents used to treat diffuse large B-cell lymphoma (DLBCL) drug therapies (Blood 2005 Abramson, J.) include cyclophosphamide, doxorubicin, vincristine, prednisone, anti-CD20 monoclonal antibodies, etoposide, bleomycin, many of the agents listed for Waldenstrom's, and any combination thereof, such as ICE and R-ICE.

Examples of other therapeutic agents used to treat chronic lymphocytic leukemia (CLL) (Spectrum, 2006, Fernandes, D.) include Chlorambucil (Leukeran), Cyclophosphamide (Cyloxan, Endoxan, Endoxana, Cyclostin), Fludarabine (Fludara), Pentstatin (Nipent), Cladribine (Leustarin), Doxorubicin (Adriamycin®, Adriblastine), Vincristine (Oncovin), Prednisone, Prednisolone, Alemtuzumab (Campath, MabCampath), many of the agents listed for Waldenstrom's, and combination chemotherapy and chemoimmunotherapy, including the common combination regimen: CVP (cyclophosphamide, vincristine, prednisone); R-CVP (rituximab-CVP); ICE (iphosphamide, carboplatin, etoposide); R-ICE (rituximab-ICE); FCR (fludarabine, cyclophosphamide, rituximab); and FR (fludarabine, rituximab).

Thus, in some aspects, provided is a method of sensitizing a subject who (i) is substantially refractory to at least one chemotherapy treatment, (ii) is in relapse after treatment with chemotherapy, or (iii) develops disease persistence to existing chronic MPN therapy, or any combination thereof, wherein the method comprises administering to the subject an effective amount of a JAK inhibitor, and an effective amount of a PI3K inhibitor or a pharmaceutically acceptable salt thereof. A subject who is sensitized is a subject who is responsive to the treatment involving administration of a JAK inhibitor and a PI3K inhibitor, or who has not developed resistance to such treatment. In one aspect, the JAK inhibitor is Compound A or ruxolitinib or pharmaceutically acceptable salt thereof, and the PI3K inhibitor is Compound B, C, D, or E, or pharmaceutically acceptable salt thereof.

The treatment involving administration of the JAK inhibitor and the PI3Kδ inhibitor, can also sensitize, or restore sensitivity of, cells that may otherwise be resistant, have developed resistance, or not responsive, to killing or apoptosis by chemotherapy treatments or by administration of a JAK inhibitor alone. The cells that are sensitized, or have restored sensitivity, are the diseased cells that are responsive to the treatment involving administration of a JAK inhibitor and a PI3Kδ inhibitor. In some embodiments, the administration of a JAK inhibitor and a PI3K inhibitor sensitizes, or restores sensitivity of, such MF cells by increasing the level of reduction in cell viability. In certain embodiments, the level of reduction in cell viability is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% compared to contact with only a JAK inhibitor alone. Also, the level of reduction in cell viability may be increased by between 10% and 99%, between 10% and 90%, between 10% and 80%, between 10% and 70%, between 20% and 99%, between 20% and 90%, between 20% and 80%, between 25% and 95%, between 25% and 90%, between 25% and 80%, between 25% and 75%, or between 30% and 90%.

In other aspects, provided is a method of sensitizing a subject who is (i) substantially refractory to at least one chemotherapy treatment, or (ii) is in relapse after treatment with chemotherapy, or both (i) and (ii), wherein the method comprises administering to the subject an effective amount of Compound B, and an effective amount of obinutuzumab. A subject who is sensitized is a subject who is responsive to the treatment involving administration of Compound B and obinutuzumab, or who has not developed resistance to such treatment.

The treatment involving administration of Compound B and obinutuzumab, can also sensitize, or restore sensitivity of, cells that may otherwise be resistant, have developed resistance, or not responsive, to killing or apoptosis by chemotherapy treatments or by administration of a PI3K-δ inhibitor (such as Compound B or Compound C) alone. Cancer cells that are sensitized, or have restored sensitivity, are cancer cells that are responsive to the treatment involving administration of Compound B and obinutuzumab, or Compound C and obinutuzumab. In some embodiments, the administration of both compounds sensitizes, or restores sensitivity of, such cancer cells by increasing the level of reduction in cell viability. In certain embodiments, the administration of Compound B and obinutuzumab, or Compound C and obinutuzumab increases the level of reduction in cell viability by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% compared to contact with only Compound B or Compound C or contact with only obinutuzumab. In other embodiments, the administration of Compound B and obinutuzumab, or Compound C and obinutuzumab increases the level of reduction in cell viability by between 10% and 99%, between 10% and 90%, between 10% and 80%, between 10% and 70%, between 20% and 99%, between 20% and 90%, between 20% and 80%, between 25% and 95%, between 25% and 90%, between 25% and 80%, between 25% and 75%, or between 30% and 90%.

Treatment

As used herein, “treatment” or “treating” is an approach for obtaining beneficial or desired results including clinical results. For example, beneficial or desired clinical results may include one or more of the following: (i) decreasing one more symptoms resulting from the disease; (ii) diminishing the extent of the disease, stabilizing the disease (e.g., preventing or delaying the worsening of the disease); (iii) preventing or delaying the spread (e.g., metastasis) of the disease; (iv) preventing or delaying the occurrence or recurrence of the disease, delay or slowing the progression of the disease; (v) ameliorating the disease state, providing a remission (whether partial or total) of the disease, decreasing the dose of one or more other medications required to treat the disease; (vi) delaying the progression of the disease, increasing the quality of life, and/or (vii) prolonging survival.

In one variation, the administration of a JAK inhibitor, such as Compound A or ruxolitinib or pharmaceutically acceptable salt thereof, and a PI3K-δ inhibitor, such as Compound B, Compound C, Compound D, or Compound E or pharmaceutically acceptable salts thereof, decreases the severity of the disease. The decrease in the severity of the disease may be assessed by chemokine levels (e.g., CCL2, CCL3, CCL4, CCL22) by the methods described herein.

Also, the administration of one or more therapeutic agent, including a JAK inhibitor and/or a PI3K-δ inhibitor, may reduce the severity of one or more symptoms associated with cancer or myeloproliferative disorder by at least about any of 10%, 20%, 30%, 40%, 50%, 60%, 70%, 80%, 90%, 95% or 100% compared to the corresponding one or more symptoms in the same subject prior to treatment or compared to the corresponding symptom in other subjects not receiving such treatment.

In another variation, the administration of Compound B and obinutuzumab, or Compound C and obinutuzumab, decreases the severity of the cancer. In one aspect, the decrease in the severity of the cancer may be assessed by chemokine levels (e.g., CCL2, CCL3, CCL4, CCL22) by the methods described herein.

Also, the administration of Compound B and obinutuzumab, or Compound C and obinutuzumab, may reduce the severity of one or more symptoms associated with cancer by at least about any of 10%, 20%, 30%, 40%, 50%, 60%, 70%, 80%, 90%, 95% or 100% compared to the corresponding one or more symptoms in the same subject prior to treatment or compared to the corresponding symptom in other subjects not receiving the composition. In certain embodiments, treatment or treating may also include a reduction of pathological consequence of cancer. The methods provided contemplate any one or more of these aspects of treatment.

As used herein, “delaying” the development of a cancer or myeloproliferative disease means to defer, hinder, slow, retard, stabilize, and/or postpone development of the disease. The delay can be of varying lengths of time, depending on the history of the disease and/or subject being treated. As is evident to one of skill in the art, a sufficient or significant delay can, in effect, encompass prevention, in that the individual does not develop the disease. A method that “delays” development of cancer or myeloproliferative disorder is a method that reduces probability of disease development in a given time frame and/or reduces the extent of the disease in a given time frame, when compared to not using the method. Such comparisons are typically based on clinical studies, using a statistically significant number of subjects. Disease development can be detectable using standard methods, such as routine physical exams, blood draw, mammography, imaging, or biopsy. Development may also refer to disease progression that may be initially undetectable and includes occurrence, recurrence, and onset.

In certain embodiments, the methods provided herein may be used to treat the growth or proliferation of cancer cells or myeloproliferative disease cells. By way of example, the cancer cells are of hematopoietic origin, myeloid, erythroid, megakaryocytic, or granulocytic, progenitors.

In other embodiments, the methods may be used to treat the growth or proliferation of cancer cells of hematopoietic origin. For example, the cancer cells may be of lymphoid origin. In one embodiment, the cancer cells are related to or derived from B lymphocytes or B lymphocyte progenitors. The administration of both Compound B and obinutuzumab, or both Compound C and obinutuzumab, may decrease cell viability of cancer cells, disrupt or inhibit phosphorylation in certain metabolic pathways, and/or reduce or inhibit certain chemokine production that may correlate with reducing disease severity.

In some aspects, also provided herein are the methods for decreasing cell viability in diseased cells in a human, comprising administering to a JAK inhibitor or a PI3Kδ inhibitor in amounts sufficient to detectably decrease cell viability in the diseased cells. The cell viability in the cancer cells after administering to the human, or contacting the diseased cells with, a JAK inhibitor and/or a PI3K inhibitor is decreased by at least 10%, at least 20%, at least 30%, at least 40%, at least 50%, at least 60%, at least 70%, at least 80%, or at least 90% compared to cell viability in the diseased cells in the absence of the inhibitors. In addition, the cell viability in diseased cells after administering to the human, or contacting the cancer cells with, a JAK inhibitor and a PI3Kδ inhibitor is decreased by between 10% and 99%, between 10% and 90%, between 10% and 80%, between 20% and 90%, between 20% and 80%, between 20% and 70% compared to cell viability in cancer cells in the absence of the inhibitors. Any suitable methods, techniques and assays known in the art may be used to measure cell viability. For example, cell viability in cancer cells is determined by flow cytometry or immunoblotting with the use of suitable stains, dyes, polynucleotide, polypeptide, or biomarkers.

In other aspects, provided herein are also methods for decreasing cell viability in cancer cells in a human, comprising administering to the human Compound B and obinutuzumab, or Compound C and obinutuzumab, in amounts sufficient to detectably decrease cell viability in the cancer cells. Provided herein are also methods for decreasing cell viability in cancer cells, comprising administering to the human or contacting the cancer cells with Compound B and obinutuzumab, or Compound C and obinutuzumab, in amounts sufficient to detectably decrease cell viability in the cancer cells. In some embodiments, the cell viability in the cancer cells after administering to the human, or contacting the cancer cells with, Compound B and obinutuzumab, or Compound C and obinutuzumab, is decreased by at least 10%, at least 20%, at least 30%, at least 40%, at least 50%, at least 60%, at least 70%, at least 80%, or at least 90% compared to cell viability in cancer cells in the absence of Compound B and obinutuzumab, or Compound C and obinutuzumab. In certain embodiments, the cell viability in cancer cells after administering to the human, or contacting the cancer cells with, Compound B and obinutuzumab, or Compound C and obinutuzumab, is decreased by between 10% and 99%, between 10% and 90%, between 10% and 80%, between 20% and 90%, between 20% and 80%, between 20% and 70% compared to cell viability in cancer cells in the absence of Compound B and obinutuzumab, or Compound C and obinutuzumab. In one embodiment of the foregoing methods, the cancer cells are chronic lymphocytic leukemia (CLL) cells.

Any suitable methods, techniques and assays known in the art may be used to measure cell viability. For example, in one embodiment, cell viability in cancer cells, such as CLL cells, may be determined by a cell viability assay, such as MTS assay. Other suitable assays may include, for example, the use of suitable stains, dyes, polynucleotide, polypeptide, or biomarkers.

In some aspects, the disclosure also provides methods for decreasing AKT phosphorylation, S6 phosphorylation, and/or ERK phosphorylation in diseased cells in a human, comprising administering to the human a JAK inhibitor or a PI3K inhibitor in amounts sufficient to detectably decrease AKT phosphorylation, S6 phosphorylation, and/or ERK phosphorylation in the diseased cells. By way of example, AKT, S6, and/or ERK phosphorylation in the diseased cells after treatment is decreased by at least 10%, at least 20%, at least 30%, at least 40%, at least 50%, at least 60%, at least 70%, at least 80%, or at least 90% compared to S6 phosphorylation in the diseased cells in the absence of the inhibitors. Additionally, AKT, S6 and/or ERK phosphorylation in the diseased cells after administering to the human, or contacting the cancer cells with, a JAK inhibitor and a PI3K inhibitor is decreased by between 10% and 99%, between 10% and 90%, between 10% and 80%, between 20% and 90%, between 20% and 80%, between 20% and 70% compared to AKT and/or S6 phosphorylation in diseased cells in the absence of the inhibitors. Any suitable methods, techniques and assays known in the art may be used to measure AKT phosphorylation, S6 phosphorylation, and ERK phosphorylation. For example, AKT phosphorylation, S6 phosphorylation, and/or ERK phosphorylation is determined by flow cytometry or immunoblotting with the use of suitable stains, dyes, polynucleotide, polypeptide, or biomarkers.

In other aspects, provided herein are also methods for decreasing AKT phosphorylation, S6 phosphorylation, or AKT and S6 phosphorylation in cancer cells in a human, comprising administering to the human Compound B and obinutuzumab, in amounts sufficient to detectably decrease AKT phosphorylation, S6 phosphorylation, or AKT and S6 phosphorylation in the cancer cells. Provided herein are also methods for decreasing AKT phosphorylation, S6 phosphorylation, or AKT and S6 phosphorylation in cancer cells, comprising administering to the human or contacting cancer cells with Compound B and obinutuzumab in amounts sufficient to detectably decrease AKT phosphorylation, S6 phosphorylation, or AKT and S6 phosphorylation in the cancer cells. In some embodiments, S6 phosphorylation in the cancer cells after administering to the human, or contacting the cancer cells with, Compound B and obinutuzumab, is decreased by at least 10%, at least 20%, at least 30%, at least 40%, at least 50%, at least 60%, at least 70%, at least 80%, or at least 90% compared to S6 phosphorylation in cancer cells in the absence of Compound B and obinutuzumab, or the absence of Compound C and obinutuzumab. In certain embodiments, S6 phosphorylation in cancer cells after administering to the human, or contacting the cancer cells with, Compound B and obinutuzumab, or Compound C and obinutuzumab is decreased by between 10% and 99%, between 10% and 90%, between 10% and 80%, between 20% and 90%, between 20% and 80%, between 20% and 70% compared to S6 phosphorylation in cancer cells in the absence of Compound B and obinutuzumab, or the absence of Compound C and obinutuzumab. In one embodiment of the foregoing methods, the cancer cells are chronic lymphocytic leukemia (CLL) cells.

Provided herein are also methods for decreasing AKT phosphorylation, ERK phosphorylation, or AKT and ERK phosphorylation in cancer cells in a human, comprising administering to a human Compound B and obinutuzumab, or Compound C and obinutuzumab, in amounts sufficient to detectably decrease AKT phosphorylation, ERK phosphorylation, or AKT and ERK phosphorylation in the cancer cells. Provided herein are also methods for decreasing AKT phosphorylation, ERK phosphorylation, or AKT and ERK phosphorylation in cancer cells, comprising contacting cancer cells with Compound B and obinutuzumab, or Compound C and obinutuzumab, in amounts sufficient to detectably decrease AKT phosphorylation, ERK phosphorylation, or AKT and ERK phosphorylation in the cancer cells. In some embodiments, ERK phosphorylation in the cancer cells after administering to the human or contacting the cancer cells with, Compound B and obinutuzumab, or Compound C and obinutuzumab, is decreased by at least 10%, at least 20%, at least 30%, at least 40%, at least 50%, at least 60%, at least 70%, at least 80%, or at least 90% compared to ERK phosphorylation in cancer cells in the absence of Compound B and obinutuzumab, or the absence of Compound C and obinutuzumab. In certain embodiments, ERK phosphorylation in cancer cells after administering to the human, or contacting the cancer cells with, Compound B and obinutuzumab, or Compound C and obinutuzumab, is decreased by between 10% and 99%, between 10% and 90%, between 10% and 80%, between 20% and 90%, between 20% and 80%, between 20% and 70% compared to ERK phosphorylation in cancer cells in the absence of Compound B and obinutuzumab, or the absence of Compound C and obinutuzumab. In one embodiment of the foregoing methods, the cancer cells are Burkitt's lymphoma cells.

Any suitable methods, techniques and assays known in the art may be used to measure AKT phosphorylation, S6 phosphorylation, and ERK phosphorylation. For example, in one embodiment, AKT phosphorylation, S6 phosphorylation, and/or ERK phosphorylation in cancer cells, such as CLL cells or Burkitt's lymphoma cells, may be determined by flow cytometry or immunoblotting.

In some aspects, provided herein also are methods for decreasing chemokine production in a sample, comprising contacting the sample with a JAK inhibitor and a PI3K inhibitor in amounts sufficient to detect chemokine production in the sample. The levels of chemokine production or expression after contact or administer with a JAK inhibitor and a PI3K inhibitor is decreased by at least 5%, at least 10%, at least 20%, at least 30%, at least 40%, at least 50%, at least 60%, at least 70%, at least 80%, or at least 90% compared to those in the cells in the absence of inhibitors. The chemokine includes but is not limited to CCL2, CCL3, CCL4, CCL22, CXCL12, CXCL13, tumor necrosis factor alpha, c-creative protein, or any combination thereof.

For example, in certain aspects, provided herein also are methods for decreasing chemokine production in a sample comprising cells expressing CCL2, CCL3, CCL4, CCL22, or any combinations thereof, comprising contacting the sample with Compound B and obinutuzumab, or Compound C and obinutuzumab, in amounts sufficient to detectably chemokine production in the sample.

In some embodiments, one or more of the following (i)-(iv) applies:

(i) CLL2 production after contact with Compound B and obinutuzumab, or Compound C and obinutuzumab, is decreased by at least 5%, at least 10%, at least 20%, at least 30%, at least 40%, at least 50%, at least 60%, at least 70%, at least 80%, or at least 90% compared to CLL2 production in the cells in the absence of Compound B and obinutuzumab, or the absence of Compound C and obinutuzumab;

(ii) CLL3 production after contact with Compound B and obinutuzumab, or Compound C and obinutuzumab, is decreased by at least 5%, at least 10%, at least 20%, at least 30%, at least 40%, at least 50%, at least 60%, at least 70%, at least 80%, or at least 90% compared to CLL3 production in the cells in the absence of Compound B and obinutuzumab, or the absence of Compound C and obinutuzumab;

(iii) CLL4 production after contact with Compound B and obinutuzumab, or Compound C and obinutuzumab, is decreased by at least 5%, at least 10%, at least 20%, at least 30%, at least 40%, at least 50%, at least 60%, at least 70%, at least 80%, or at least 90% compared to CLL4 production in the cells in the absence of Compound B and obinutuzumab, or the absence of Compound C and obinutuzumab; and

(iv) CLL22 production after contact with Compound B and obinutuzumab, or Compound C and obinutuzumab, is decreased by at least 5%, at least 10%, at least 20%, at least 30%, at least 40%, at least 50%, at least 60%, at least 70%, at least 80%, or at least 90% compared to CLL22 production in the cells in the absence of Compound B and obinutuzumab, or the absence of Compound C and obinutuzumab.

It is intended and understood that each and every variation of the decrease in production of any one of the chemokines provided above may be combined with each and every variation of the other chemokines, as if each and every combination is individually described. For example, in some variations, CCL3, CCL4, CXCL12, CXCL13, tumor necrosis factor alpha, and c-creative protein may be suitable chemokines.

Any suitable methods, techniques and assays known in the art may be used to determine the levels of the chemokines in a sample. For example, immunoassays (or immunological binding assays) may be used to qualitatively or quantitatively analyze the chemokine levels in a sample. A general overview of the applicable technology can be found in a number of readily available manuals, e.g., Harlow & Lane, Cold Spring Harbor Laboratory Press, Using Antibodies: A Laboratory Manual (1999) Immunoassays typically use an antibody that specifically binds to a protein or antigen of choice. The antibody may be produced by any of a number of means well known to those of skill in the art.

For in vitro or in vivo studies, the effect amount of Compounds A, B, C, D, E, or ruxolinitib may be adjusted according to the experimental condition. For example, compounds may be used in the amount of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 5.0, 6.0, 7.0, 8.0, 9.0, or 10.0 μM.

### Dosing Regimen, Order of Administration, and Route of Administration

As used herein, a “therapeutically effective amount” means an amount sufficient to modulate JAK/STAT and/or PI3K pathways, and thereby treat a subject (such as a human) suffering an indication, or to alleviate the existing symptoms of the indication. Determination of a therapeutically effective amount is within the capability of those skilled in the art, especially in light of the detailed disclosure provided herein. In some embodiments, a therapeutically effective amount of a JAK inhibitor, such as Compound A or ruxolitinib or pharmaceutically acceptable salt thereof, and a therapeutically effective amount of PI3K inhibitor, such as Compound B, Compound C, Compound D, or Compound E and pharmaceutically acceptable salt thereof, may (i) reduce the number of diseased cells; (ii) reduce tumor size; (iii) inhibit, retard, slow to some extent, and preferably stop the diseased cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with cancer or myeloproliferative disease. In other embodiments, a therapeutically effective amount of Compound B or Compound C and a therapeutically effective amount of obinutuzumab may (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

The dosing regimen of the inhibitors according to the present disclosure may vary depending upon the indication, route of administration, and severity of the condition, for example, depending on the route of administration, a suitable dose can be calculated according to body weight, body surface area, or organ size. The final dosing regimen is determined by the attending physician in view of good medical practice, considering various factors that modify the action of drugs, e.g., the specific activity of the compound, the identity and severity of the disease state, the responsiveness of the patient, the age, condition, body weight, sex, and diet of the patient, and the severity of any infection. Additional factors that can be taken into account include time and frequency of administration, drug combinations, reaction sensitivities, and tolerance/response to therapy. Further refinement of the doses appropriate for treatment involving any of the formulations mentioned herein is done routinely by the skilled physician or practitioner without undue experimentation, especially in light of the dosing information and assays disclosed, as well as the pharmacokinetic data observed in human clinical trials. Appropriate doses can be ascertained through use of established assays for determining concentration of the agent in a body fluid or other sample together with dose response data.

The formulation and route of administration chosen may be tailored to the individual subject, the nature of the condition to be treated in the subject, and generally, the judgment of the attending practitioner. For example, the therapeutic index of the inhibitors described herein may be enhanced by modifying or derivatizing the compound for targeted delivery to the diseased cells expressing a marker that identifies the cells as such. For example, the compounds can be linked to an antibody that recognizes a marker that is selective or specific for cancer cells, so that the compounds are brought into the vicinity of the cells to exert their effects locally, as previously described. See e.g., Pietersz et al., Immunol. Rev., 129:57 (1992); Trail et al., Science, 261:212 (1993); and Rowlinson-Busza et al., Curr. Opin. Oncol., 4:1142 (1992).

Dosing Regimen

The therapeutically effective amount of a JAK inhibitor, such as Compound A or ruxolitinib or pharmaceutically acceptable salt thereof, or a PI3K inhibitor, such as Compound B, Compound C, Compound D, or Compound E or pharmaceutically acceptable salts thereof, may be provided in a single dose or multiple doses to achieve the desired treatment endpoint. The therapeutically effective amount of Compound B or obinutuzumab, or Compound C and obinutuzumab may also be provided in a single dose or multiple doses to achieve the desired treatment endpoint. As used herein, “dose” refers to the total amount of an active ingredient (e.g., Compound A, Compound B, Compound C, Compound D, Compound E, or pharmaceutically acceptable salts thereof) to be taken each time by a subject (e.g., a human); or Compound B or Compound C, obinutuzumab to be taken each time by a subject (e.g., a human)).

In some variations, exemplary doses of the compounds of the present disclosure may be between about 20 mg to about 1000 mg, or between about 20 mg to about 500 mg, or between about 25 mg to about 400 mg, or between about 50 mg to about 350 mg, or between about 75 mg to about 300 mg, or between about 100 mg to about 200 mg, or about 10 mg, or about 15 mg, or about 20 mg, or about 25 mg, or about 30 mg, or about 40 mg, or about 50 mg, or about 60 mg, or about 75 mg, or about 100 mg, or about 125 mg, or about 150 mg, or about 175 mg, or about 200 mg, or about 225 mg, or about 250 mg, or about 275 mg, or about 300 mg, or about 325 mg, or about 350 mg, or about 375 mg, or about 400 mg, or about 425 mg, or about 450 mg, or about 475 mg, or about 500 mg. It should be understood that reference to “about” a value or parameter herein includes (and describes) embodiments that are directed to that value or parameter per se. For example, description referring to “about x” includes description of “x” per se.

In certain variations, exemplary doses of Compound B or Compound C, for a human subject may be between about 0.01 mg to about 1500 mg or between about 50 mg to about 200 mg, or about 200 mg to about 300 mg or about 75 mg, or about 100 mg, or about 125 mg, or about 150 mg, or about 175 mg, or about 200 mg, or about 225 mg, or about 250 mg, or about 275 mg, or about 300 mg, or about 325 mg, or about 350 mg, or about 375 mg, or about 400 mg, or about 425 mg, or about 450 mg, or about 475 mg, or about 500 mg. It should be understood that reference to “about” a value or parameter herein includes (and describes) embodiments that are directed to that value or parameter per se. For example, description referring to “about x” includes description of “x” per se.

In certain variations, exemplary doses of obinutuzumab, for a human subject may be between about 100 mg to about 5000 mg, or about 500 mg to about 200 mg, or about 100 mg, or about 200 mg, or about 300 mg, or about 400 mg, or about 500 mg, or about 600 mg, or about 700 mg, or about 800 mg, or about 900 mg, or about 1000 mg, or about 1100 mg, or about 1200 mg, or about 1300 mg, or about 1400 mg, or about 1500 mg, or about 1600 mg, or about 1700 mg, or about 1800 mg, or about 1900 mg, or about 2000 mg, or about 2500 mg, or about 3000 mg, or about 3500 mg, or about 4000 mg, or about 4500 mg, or about 5000 mg.

Each and every variation of the doses of a JAK inhibitor, such as Compound A or ruxolitinib or pharmaceutically acceptable salt thereof, may be combined with each and every variation of the doses of a PI3K inhibitor, such as Compound B, Compound C, Compound D, Compound E or pharmaceutically acceptable salt thereof, as if each and every combination is individually described. For example, a 25 mg dose of a JAK inhibitor may be administered with a PI3K inhibitor at a dose of 100, 125, 150, 175, 200, 225, 250, 275, 300, 325, 350, 375, or 400 mg. In some example, a 100 mg dose of a JAK inhibitor may be administered with a PI3K inhibitor at a dose of 100, 125, 150, 175, 200, 225, 250, 275, 300, 325, 350, 375, or 400 mg. In additional example, a 200 mg dose of a JAK inhibitor may be administered with a PI3K inhibitor at a dose of 100, 125, 150, 175, 200, 225, 250, 275, 300, 325, 350, 375, or 400 mg. Additional example includes that a 300 mg dose of a JAK inhibitor may be administered with a PI3K inhibitor at a dose of 100, 125, 150, 175, 200, 225, 250, 275, 300, 325, 350, 375, or 400 mg. In one embodiment, 200 mg of Compound A and 100 mg of Compound B or 200 mg of Compound A and 150 mg of Compound B are used in the methods or present disclosure.

Each and every variation of the doses of Compound B or Compound C may be combined with each and every variation of the doses of obinutuzumab, as if each and every combination is individually described. For example, in one embodiment, a 100 mg dose of Compound B or Compound C may be administered with a 1000 mg dose of obinutuzumab. In another embodiment, a 150 mg dose of Compound B or Compound C may be administered with a 1000 mg dose of obinutuzumab. In yet another embodiment, a 200 mg dose of Compound B or Compound C may be administered with a 1000 mg dose of obinutuzumab. In other embodiment, a 300 mg dose of Compound B or Compound C may be administered with a 1000 mg dose of obinutuzumab. In another embodiment, a 75 mg dose of Compound B or Compound C may be administered with a 1000 mg dose of obinutuzumab.

In other embodiments, the methods provided comprise continuing to treat the subject (e.g., a human) by administering the doses of inhibitors or compounds at which clinical efficacy is achieved or reducing the doses by increments to a level at which efficacy can be maintained. In a particular embodiment, the methods provided herein comprise administering to the subject (e.g., a human) an initial daily dose of 100 mg to 200 mg of the compound, and increasing said dose to a total dosage of 100 mg to 400 mg per day over at least 6 days. Optionally, the dosage can be further increased to about 150-750 mg per day. The dose(s) of Compound A, Compound B, Compound C, Compound D and/or Compound E, or pharmaceutically acceptable salts thereof, may be increased by increments until clinical efficacy is achieved. Increments of about 100 mg, or about 125 mg, or about 150 mg, or about 200 mg, or about 250 mg, or about 300 mg, or about 400 mg can be used to increase the dose. The dose can be increased daily, every other day, two, three, four, five or six times per week, or once per week.

The frequency of dosing will depend on the pharmacokinetic parameters of the compounds administered and the route of administration. The dosing frequency for the JAK inhibitor may be the same or different from the dosing frequency for the PI3K inhibitor. The JAK inhibitor, such as Compound A or ruxolitinib or pharmaceutically acceptable salt thereof, is administered once a day or twice a day. Also, the PI3K inhibitor, such as Compounds B, C, D, E or a pharmaceutically acceptable salt thereof, is administered once a day or twice a day. The administration of the JAK inhibitor and the administration of PI3K inhibitor may be together or separately. The dosing frequency for Compound B or Compound C may be the same or different from the dosing frequency for obinutuzumab. In some embodiments, Compound B or Compound C or a pharmaceutically acceptable salt thereof is administered once a day or twice a day. In some embodiments, Compound B or Compound C or a pharmaceutically acceptable salt thereof is administered once a day. In some embodiments, Compound B or Compound C or a pharmaceutically acceptable salt thereof is administered twice a day. In some embodiments, obinutuzumab is administered once a week or once every two weeks. In some embodiments, obinutuzumab is administered in eight (8) doses over a period of 21 weeks. In some embodiments, obinutuzumab is administered once every 28 days. In some embodiments, Compound B or Compound C or a pharmaceutically acceptable salt thereof is administered once a day and obinutuzumab is administered once every 28 days. In some embodiments, obinutuzumab is administered once every 28 days. In some embodiments, Compound B or Compound C or a pharmaceutically acceptable salt thereof is administered twice a day and obinutuzumab is administered once every 28 days.

The dose and frequency of dosing also depend on pharmacokinetic and pharmacodynamic, as well as toxicity and therapeutic efficiency data. For example, pharmacokinetic and pharmacodynamic information about the compound of the present disclosure can be collected through preclinical in vitro and in vivo studies, later confirmed in humans during the course of clinical trials. In another example, pharmacokinetic and pharmacodynamic information about Compound B and obinutuzumab, or Compound C and obinutuzumab, and the formulation of Compound B and obinutuzumab, or Compound C and obinutuzumab can be collected through preclinical in vitro and in vivo studies, later confirmed in humans during the course of clinical trials. Thus, a therapeutically effective dose can be estimated initially from biochemical and/or cell-based assays. Then, dosage can be formulated in animal models to achieve a desirable circulating concentration range that modulates PI3Kδ and/or expression or activity. As human studies are conducted further information will emerge regarding the appropriate dosage levels and duration of treatment for various diseases and conditions.

Toxicity and therapeutic efficacy (e.g., of Compound A and Compound B; ruxolitinib and Compound B; Compound B and obinutuzumab; and Compound C and obinutuzumab) can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the “therapeutic index”, which typically is expressed as the ratio LD₅₀/ED₅₀. Compounds that exhibit large therapeutic indices, i.e., the toxic dose is substantially higher than the effective dose, are preferred. The data obtained from such cell culture assays and additional animal studies can be used in formulating a range of dosage for human use. The doses of such compounds lies preferably within a range of circulating concentrations that include the ED₅₀ with little or no toxicity.

Compounds A, B, C, D, E or pharmaceutically acceptable salts thereof may be administered under fed conditions. For example, in some variations, Compound B and obinutuzumab, or Compound C and obinutuzumab may be administered under fed conditions. The term fed conditions or variations thereof refers to the consumption or uptake of food, in either solid or liquid forms, or calories, in any suitable form, before or at the same time when the compounds or pharmaceutical compositions thereof are administered. Compound may be administered to the subject (e.g., a human) within minutes or hours of consuming calories (e.g., a meal). By way of example, the JAK inhibitor and/or the PI3K inhibitor is administered to the subject (e.g., a human) within 5-10 minutes, about 30 minutes, or about 60 minutes consuming calories.

Order of Administration

The order of administering according to the present disclosure may also vary. The compounds may be administered sequentially (e.g., sequential administration) or simultaneously (e.g., simultaneous administration). For example, the JAK inhibitor is administered before the PI3K inhibitor, or the PI3K inhibitor is administered before the JAK inhibitor. Also, in some variations, the JAK inhibitor and the PI3K inhibitor are administered simultaneously. In another example, Compound B or Compound C or a pharmaceutically acceptable salt thereof is administered before obinutuzumab. In other embodiments, obinutuzumab is administered before Compound B or Compound C or a pharmaceutically acceptable salt thereof. In yet other embodiments, Compound B or Compound C or a pharmaceutically acceptable salt thereof, and obinutuzumab, are administered simultaneously. Further, the administration of the compounds can be combined with supplemental doses.

Sequential administration or administered sequentially means that the inhibitors, compounds, or drugs are administered with a time separation of several minutes, hours, days, or weeks. Compounds may be administered with a time separation of at least 15 minutes, at least 30 minutes, at least 60 minutes, or 1 day, 2 days, 3 days, 4 days, 5 days, 6 days, or 7 days, or 2 weeks, 3 weeks, 4 weeks, 5 weeks, 6 weeks, 7 weeks, or 8 weeks. When administered sequentially, the compounds or drugs may be administered in two or more administrations, and the compounds or drugs are contained in separate compositions which may be contained in the same or different packages.

Simultaneous administration or administered simultaneously means that the inhibitors, compounds, or drugs are administered with a time separation of no more than a few minutes or seconds. Compounds are administered with a time separate of no more than about 15 minutes, about 10 minutes, about 5 minutes, or 1 minute. When administered simultaneously, the inhibitors, compounds or drugs are contained in separate compositions or the same composition.

The present disclosure shows that the administration of a JAK inhibitor and a PI3Kδ inhibitor provide unexpected synergy or synergistic effect(s). The present disclosure also shows that the administration of an anti-CD20 antibody and a PI3Kδ inhibitor provide unexpected synergy or synergistic effect(s). As used herein, synergy or synergistic effects means the effect achieved when the active ingredients used together is greater than the sum of the effects that results from using the compounds separately or greater than the additive effects resulted from the compound alone. A synergistic effect may be attained when the active ingredients are: (1) co-formulated and administered or delivered simultaneously in a combined formulation; (2) delivered sequentially or simultaneously as separate formulations; or (3) by some other regimen. In certain embodiments, a synergistic effect may be attained when the compounds are administered or delivered sequentially, e.g., in separate tablets, pills or capsules, or by different injections in separate syringes.

Modes of Administration

Compounds according to the present disclosure may be administered by any conventional method, including parenteral and enteral techniques. For example, in some variations, Compound B and obinutuzumab, or Compound C and obinutuzumab, may be administered by any conventional method, including parenteral and enteral techniques. Parenteral administration modalities include those in which the composition is administered by a route other than through the gastrointestinal tract, for example, intravenous, intraarterial, intraperitoneal, intramedullary, intramuscular, intraarticular, intrathecal, and intraventricular injections. Enteral administration modalities include, for example, oral, buccal, sublingual, and rectal administration. Transepithelial administration modalities include, for example, transmucosal administration and transdermal administration. Transmucosal administration includes, for example, enteral administration as well as nasal, inhalation, and deep lung administration; vaginal administration; and buccal and sublingual administration. Transdermal administration includes passive or active transdermal or transcutaneous modalities, including, for example, patches and iontophoresis devices, as well as topical application of pastes, salves, or ointments. Parenteral administration also can be accomplished using a high-pressure technique, e.g., POWDERJECT™.

By way of example, the JAK inhibitor and the PI3K inhibitor are independently administered orally, intravenously or by inhalation. In one embodiment, the JAK inhibitor is administered orally, once or twice, at a dosage of about 10 mg, about 20 mg, about 25 mg, about 30 mg, about 40 mg, about 50 mg, about 75 mg, about 100 mg, about 150 mg, about 200 mg, about 225 mg, about 250 mg, about 275 mg, about 300 mg, about 350 mg, about 400 mg, about 450 mg, about 500 mg, about 550 mg, or about 600 mg. In other embodiment, the PI3K inhibitor is administered orally, once or twice, at a dosage of about about 100 mg, about 150 mg, about 200 mg, about 225 mg, about 250 mg, about 275 mg, about 300 mg, about 350 mg, about 400 mg, about 450 mg, about 500 mg, about 550 mg, or about 800 mg.

In some embodiments, Compound B and obinutuzumab, or Compound C and obinutuzumab, may be independently administered orally, intravenously or by inhalation. In one embodiment, Compound B or Compound C, or both, are administered orally and obintuzumab is administered parenterally. In one embodiment, Compound B or Compound C, or both, are administered orally and obintuzumab is administered by intravenous infusion.

In one embodiment, Compound B or Compound C, or a pharmaceutically acceptable salt thereof, is administered orally. In some embodiments, Compound B or Compound C is administered orally at a dosage of about 50 mg BID, about 100 mg BID, about 150 mg BID, about 200 mg, about 225 mg, about 250 mg, about 275 mg, about 300 mg, about 350 mg, about 400 mg, about 450 mg, about 500 mg, about 550 mg, about 600 mg, about 650 mg, or about 700 mg BID, or about 800 mg, or about 900 mg, or about 1100 mg, or about 1200 mg. In some embodiments, Compound B or Compound C is administered orally at a dosage of about 50 mg BID, about 100 mg BID, or about 150 mg BID. In some embodiments, Compound B or Compound C is administered orally at a dosage of about 75 mg BID. In some embodiments, Compound B or Compound C is administered orally at a dosage of about 50 mg QD, about 100 mg QD, about 150 mg QD, about 200 mg, about 225 mg QD, about 250 mg QD, about 275 mg QD, about 300 mg QD, about 350 mg QD, about 400 mg QD, about 450 mg QD, about 500 mg QD, about 550 mg QD, about 600 mg QD, about 650 mg QD, or about 700 mg QD, or about 800 mg QD, or about 900 mg QD, or about 1100 mg QD, or about 1200 mg QD. In some embodiments, Compound B or Compound C is administered orally at a dosage of about 50 mg BID, about 100 mg BID, about 150 mg BID, about 200 mg, about 225 mg BID, about 250 mg BID, about 275 mg BID, about 300 mg BID, about 350 mg BID, about 400 mg BID, about 450 mg BID, about 500 mg BID, about 550 mg BID, about 600 mg BID, about 650 mg BID, or about 700 mg BID, or about 800 mg BID, or about 900 mg BID, or about 1100 mg BID, or about 1200 mg BID.

In one embodiment, obinutuzumab is administered intravenously. In some embodiments, obinutuzumab, is administered intravenosly at a dosage of about 1000 mg per day of treatment cycle, for a period of at least about 5 treatment cycles.

Pharmaceutical Compositions

The one or more therapeutic agent can each be administered or provided as the neat chemical, but it is typical, and preferable, to administer or provide the compounds in the form of a pharmaceutical composition or formulation. Accordingly, provided are pharmaceutical compositions that include the compound within the present disclosure and a biocompatible pharmaceutical vehicle (e.g., carrier, adjuvant, and/or excipient). For example, in one variation, provided are pharmaceutical compositions that include Compound B and/or obinutuzumab, or Compound C and/or obinutuzumab and a biocompatible pharmaceutical vehicle (e.g., carrier, adjuvant, and/or excipient). The composition can include the compounds as the sole active agent(s) or in combination with other agents, such as oligo- or polynucleotides, oligo- or polypeptides, drugs, or hormones mixed with one or more pharmaceutically acceptable vehicles. In certain embodiments, pharmaceutically acceptable vehicles include pharmaceutically acceptable carriers, adjuvants and/or excipients, and other ingredients can be deemed pharmaceutically acceptable insofar as they are compatible with other ingredients of the formulation and not deleterious to the recipient thereof.

In certain embodiments, the compounds are administered in the same or separate formulations. For example, in some variations, Compound B and obinutuzumab, or Compound C and obinutuzumab, are administered in the same or separate formulations. In certain embodiments, Compound B or Compound C or a pharmaceutically acceptable salt thereof is present in a pharmaceutical composition comprising Compound B or Compound C or a pharmaceutically acceptable salt thereof, and at least one pharmaceutically acceptable vehicle. In certain embodiments, obinutuzumab is present in a pharmaceutical composition comprising obinutuzumab, and at least one pharmaceutically acceptable vehicle. In one embodiment, the active ingredients (e.g., Compound B and obinutuzumab, or Compound C and obinutuzumab) are administered in separate unit dosages (e.g., in separate tablets, pills or capsules, or by different injections in separate syringes).

The pharmaceutical composition comprises the active ingredient or the compound of the present disclosure and at least one pharmaceutically acceptable vehicle. Techniques for formulation and administration of pharmaceutical compositions can be found in Remington's Pharmaceutical Sciences, 18th Ed., Mack Publishing Co, Easton, Pa., 1990; and Modern Pharmaceutics, Marcel Dekker, Inc. 3rd Ed. (G. S. Banker & C. T. Rhodes, Eds.). The pharmaceutical compositions described herein can be manufactured using any conventional method, e.g., mixing, dissolving, granulating, dragee-making, levigating, emulsifying, encapsulating, entrapping, melt-spinning, spray-drying, or lyophilizing processes. An optimal pharmaceutical formulation can be determined by one of skill in the art depending on the route of administration and the desired dosage. Such formulations can influence the physical state, stability, rate of in vivo release, and rate of in vivo clearance of the administered agent. Depending on the condition being treated, these pharmaceutical compositions can be formulated and administered systemically or locally.

The pharmaceutical compositions can be formulated to contain suitable pharmaceutically acceptable vehicles, which may include, for example, inert solid diluents and fillers, diluents, including sterile aqueous solution and various organic solvents, permeation enhancers, solubilizers and adjuvants. For example, the pharmaceutical compositions may comprise pharmaceutically acceptable carriers, and optionally can comprise excipients and auxiliaries that facilitate processing of the compound or active ingredient into preparations that can be used pharmaceutically. In another example, the pharmaceutical compositions may comprise pharmaceutically acceptable carriers, and optionally can comprise excipients and auxiliaries that facilitate processing of the compound or the active ingredient into preparations that can be used pharmaceutically. The mode of administration generally determines the nature of the carrier. For example, formulations for parenteral administration can include aqueous solutions of the active compounds in water-soluble form. Carriers suitable for parenteral administration can be selected from among saline, buffered saline, dextrose, water, and other physiologically compatible solutions. In one embodiment, carriers for parenteral administration include physiologically compatible buffers such as Hanks's solution, Ringer's solution, or physiologically buffered saline. For tissue or cellular administration, penetrants appropriate to the particular barrier to be permeated are used in the formulation. Such penetrants are generally known in the art. For preparations including proteins, the formulation can include stabilizing materials, such as polyols (e.g., sucrose) and/or surfactants (e.g., nonionic surfactants), and the like.

Alternatively, formulations for parenteral use can include dispersions or suspensions prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxymethylcellulose, sorbitol, dextran, and mixtures thereof. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, e.g., methacrylic polymers, such as the EUDRAGIT™ series available from Rohm America Inc. (Piscataway, N.J.). Emulsions, e.g., oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

Liposomes containing the inhibitors or the compounds also can be employed for parenteral administration. Liposomes generally are derived from phospholipids or other lipid substances. The compositions in liposome form also can contain other ingredients, such as stabilizers, preservatives, excipients, and the like. Preferred lipids include phospholipids and phosphatidyl cholines (lecithins), both natural and synthetic. Methods of forming liposomes are known in the art. See, e.g., Prescott (Ed.), Methods in Cell Biology, Vol. XIV, p. 33, Academic Press, New York (1976).

In certain embodiments, the compounds of the present disclosure may be formulated for oral administration using pharmaceutically acceptable carriers well known in the art. For example, in some embodiments, Compound B, obinutuzumab, or both Compound B and obinutuzumab, or the composition thereof, are formulated for oral administration using pharmaceutically acceptable carriers well known in the art. In other embodiments, Compound C, obinutuzumab, or both Compound C and obinutuzumab, or the composition thereof, are formulated for oral administration using pharmaceutically acceptable carriers well known in the art. Preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets or dragee cores. Oral formulations can employ liquid carriers similar in type to those described for parenteral use, e.g., buffered aqueous solutions, suspensions, and the like.

In some embodiments, oral formulations include tablets, dragees, and gelatin capsules. These preparations can contain one or more excipients including but not limited to: (i) diluents, such as microcrystalline cellulose and sugars, including lactose, dextrose, sucrose, mannitol, or sorbitol; (ii) binders, such as sodium starch glycolate, croscarmellose sodium, magnesium aluminum silicate, starch from corn, wheat, rice, potato, etc.; (iii) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen; (iv) disintegrating or solubilizing agents such as cross-linked polyvinyl pyrrolidone, starches, agar, alginic acid or a salt thereof, such as sodium alginate, or effervescent compositions; (v) lubricants, such as silica, talc, stearic acid or its magnesium or calcium salt, and polyethylene glycol; (vi) flavorants and sweeteners; (vii) colorants or pigments, e.g., to identify the product or to characterize the quantity (dosage) of active compound; and (viii) other ingredients, such as preservatives, stabilizers, swelling agents, emulsifying agents, solution promoters, salts for regulating osmotic pressure, and buffers.

Gelatin capsules may include push-fit capsules made of gelatin, as well as soft, sealed capsules made of gelatin and a coating such as glycerol or sorbitol. Push-fit capsules can contain the active ingredient(s) mixed with fillers, binders, lubricants, and/or stabilizers, etc. In soft capsules, the active compounds can be dissolved or suspended in suitable fluids, such as fatty oils, liquid paraffin, or liquid polyethylene glycol with or without stabilizers.

Dragee cores may be provided with suitable coatings such as concentrated sugar solutions, which also can contain gum arabic, talc, polyvinyl pyrrolidone, carbopol gel, polyethylene glycol, and/or titanium dioxide, lacquer solutions, and suitable organic solvents or solvent mixtures.

In some aspects, provided herein are also unit dosage forms of an anti-CD20 inhibitor and a PI3K inhibitor. In other aspects, provided herein are also unit dosage forms of Compound B and obinutuzumab, or Compound C and obinutuzumab.

Articles of Manufacture and Kits

Compositions (including, for example, formulations and unit dosages) comprising the inhibitors or the compounds can be prepared and placed in an appropriate container, and labeled for treatment of an indicated condition.

Accordingly, in some aspects, provided is also an article of manufacture, such as a container comprising a unit dosage form of the compound, and a label containing instructions for use of the compounds. In some embodiments, the article of manufacture is a container comprising (i) a unit dosage form of a JAK inhibitor and one or more pharmaceutically acceptable carriers, adjuvants or excipients; and (ii) a unit dosage form of a PI3K inhibitor and one or more pharmaceutically acceptable carriers, adjuvants or excipients. In other aspects, the article of manufacture is a container comprising (i) a unit dosage form of an anti-CD20 antibody and one or more pharmaceutically acceptable carriers, adjuvants or excipients; and (ii) a unit dosage form of a PI3K inhibitor and one or more pharmaceutically acceptable carriers, adjuvants or excipients. In other aspects, the article of manufacture is a container comprising (i) a unit dosage form of an anti-CD20 antibody and one or more pharmaceutically acceptable vehicles; and (ii) a unit dosage form of a PI3K inhibitor and one or more pharmaceutically acceptable vehicles. In some embodiments, provided is also an article of manufacture, such as a container comprising a unit dosage form of Compound B or Compound C and a unit dosage form of obinutuzumab, and a label containing instructions for use of the compounds. In some embodiments, the article of manufacture is a container comprising (i) a unit dosage form of Compound B or Compound C and one or more pharmaceutically acceptable carriers, adjuvants or excipients; and (ii) a unit dosage form of obinutuzumab and one or more pharmaceutically acceptable carriers, adjuvants or excipients. In some embodiments, the article of manufacture is a container comprising (i) a unit dosage form of Compound B or Compound C and one or more pharmaceutically acceptable vehicles; and (ii) a unit dosage form of obinutuzumab and one or more pharmaceutically acceptable vehicles. In one embodiment, the unit dosage form for Compound B is a tablet. In one embodiment, the unit dosage form for Compound C is a tablet. In one embodiment, the unit dosage form for both Compound B and obinutuzumab is a tablet. In another embodiment, the unit dosage form for both Compound C and obinutuzumab is a tablet.

As used herein, “unit dosage form” refers to physically discrete units, suitable as unit dosages, each unit containing a predetermined quantity of active ingredient, or compound which may be in a pharmaceutically acceptable carrier. One of skill in the art would recognize that the unit dosage form may vary depending on the mode of administration. Exemplary unit dosage levels for a human subject may be between about 100 mg to about 1000 mg, or between 100 mg to about 400 mg, or between about 100 mg to about 300 mg, or between about 150 mg to about 200 mg, or about 100 mg, about 125 mg, or about 150 mg, or about 175 mg, about 200 mg, or about 250 mg, about 300 mg, about 350 mg, about 400 mg, about 500 mg, about 600 mg, about 700 mg, or about 800 mg. In some embodiments, the unit dosage level for a human subject is between about 75 mg to about 150 mg.

Exemplary unit dosage levels of Compound B or Compound C, or a pharmaceutically acceptable salt thereof, for a human subject may be between about 0.01 mg to about 1000 mg, or between about 50 mg to about 200 mg, or about 25 mg, about 50 mg, about 75 mg, about 100 mg, about 125 mg, or about 150 mg, or about 175 mg, about 200 mg, or about 250 mg.

Exemplary unit dosage levels of obinutuzumab, for a human subject may be between about 0.01 mg to about 1600 mg, or between about 50 mg to about 200 mg, or about 25 mg, about 50 mg, about 75 mg, about 100 mg, about 125 mg, or about 150 mg, or about 175 mg, about 200 mg, about 250 mg, about 300 mg, about 350 mg, about 400 mg, about 600 mg, about 900 mg, or about 1200 mg.

Compound B, obinutuzumab, or Compound C or pharmaceutically acceptable salts thereof may be administered as one or more unit dosage forms. For example, in one embodiment, a dose of 100 mg of Compound B or Compound C may be orally administered to a subject (e.g., a human subject) in one 100 mg tablet. In one embodiment, a dose of 200 mg of obinutuzumab may be orally administered to a subject (e.g., a human subject) in one 200 mg tablet. In another embodiment, a dose of 600 mg of obinutuzumab may be orally administered to a subject (e.g., a human subject) in three 200 mg tablets.

Kits also are contemplated. For example, a kit can comprise unit dosage forms of the compounds, and a package insert containing instructions for use of the composition in treatment of a medical condition. In some embodiments, the kit comprises (i) a unit dosage form of the JAK inhibitor and one or more pharmaceutically acceptable carriers, adjuvants or excipients; and (ii) a unit dosage form of the PI3K inhibitor and one or more pharmaceutically acceptable carriers, adjuvants or excipients. In another example, a kit can comprise unit dosage forms of Compound B and obinutuzumab, or Compound C and obinutuzumab, and a package insert containing instructions for use of the composition in treatment of a medical condition. In some embodiments, the kits comprises (i) a unit dosage form of Compound B or Compound C and one or more pharmaceutically acceptable carriers, adjuvants or excipients; and (ii) a unit dosage form of obinutuzumab and one or more pharmaceutically acceptable carriers, adjuvants or excipients. In one embodiment, the unit dosage form for both Compound B and obinutuzumab is a tablet. In another embodiment, the unit dosage form for both Compound C and obinutuzumab is a tablet.

In some variations, the instructions for use in the kit may be for treating a cancer or a myeloproliferative disorder. In other variations, the instructions for use in the kit may also be for treating a cancer, including, for example, a hematologic malignancy. In some embodiments, the instructions for use in the kit may be for treating cancer, such as leukemia or lymphoma, including relapsed and refractory leukemia or lymphoma. In certain embodiments, the instructions for use in the kit may be for treating acute lymphocytic leukemia (ALL), B-cell ALL, acute myeloid leukemia (AML), chronic lymphocytic leukemia (CLL), small lymphocytic lymphoma (SLL), multiple myeloma (MM), non-Hodgkin's lymphoma (NHL), indolent NHL (iNHL), mantle cell lymphoma (MCL), follicular lymphoma, Waldenstrom's macroglobulinemia (WM), B-cell lymphoma, or diffuse large B-cell lymphoma (DLBCL), polycythemia vera (PV), primary myelofibrosis (PMF), thrombocythemia, essential thrombocythemia (ET), idiopathic myelofibrosis (IMF), chronic myelogenous leukemia (CML), systemic mastocystosis (SM), chronic neutrophilic leukemia (CNL), myelodysplastic syndrome (MDS) and systemic mast cell disease (SMCD). In one embodiment, the instructions for use in the kit may be for treating non-Hodgkin's lymphoma (NHL) or chronic lymphocytic leukemia (CLL). In certain embodiments, conditions indicated on the label can include, for example, treatment of cancer.

### Examples

### Example 1

### Effects of Compound B to PI3K Isoforms and AKT Phosphorylation

The effects of Compound B on the activities of class I PI3K isoforms were measured using an in vitro biochemical enzyme assay at steady-state concentrations of adenosine triphosphate (ATP). Compound B is (S)-2-(1-((9H-purin-6-yl)amino)propyl)-5-fluoro-3-phenylquinazolin-4(3H)-one as described above.

A time resolved fluorescence resonance energy transfer (TR-FRET) assay was used to monitor the formation of 3,4,5-inositol triphosphate (PIP₃) molecule, as it competed with fluorescently labeled PIP₃ for binding to the GRP-1 pleckstrin homology domain protein. The Results show that Compound B was a selective inhibitor to PI3Kδ. The inhibition to PI3Kδ was 450-fold compared to PI3Kα, 210-fold compared to PI3Kβ, and 110-fold compared to PI3Kγ.

In addition, Compound B was examined for the effects on the PI3K signaling pathway by determining the levels of AKT and S6 phosphorylation with or without TPO activation. Two cell lines, BaF3/MPL and UT-7/TPO sensitive or responsive to TPO activation were used. The cells were starved (i.e. growing on medium having less FBS) in 0/1% FBS/RPMI for two hours before treated with 0.1, 1.0, or 2.0 μM of Compound B or vehicle (0.1% DMSO in RPMI) for 2 hours at 37° C. To examine the TPO-activated phosphorylation, the cells were then treated or activated with 50 ng/mL of human recombinant TPO (Peprotech) for 10 minutes at 37° C. The TPO activation or treatment may reflect the conditions in diseased cells as the PI3K pathway is activated by TPO in myelofibrosis. After treating with compound and/or TPO, the cells were collected, lysed by lysis buffer (Cell Signaling), separated by SDS-PAGE, and analyzed by the Western blot using antibodies specific to p-AKT Ser473 or pS6 Ser235/236 (Cell Signaling). The phosphorylation levels in treated cells were calculated and compared to those of untreated cells (i.e. vehicle as negative control).

The results showed that the cells treated with Compound B exhibited the reduced AKT (p-AKT Ser473) and S6 (p-S6RP Ser235/236) phosphorylation. The BaF3/MPL cells treated with 0.1, 1.0, or 2.0 μM of Compound B and TPO exhibited reduced p-AKT levels of 51%, 64%, or 67%, respectively, and reduced p-S6 levels of 24%, 27%, or 41%, respectively, of those in the cells treated with vehicle. Moreover, the U7-7/TPO cells treated with 0.1, 1.0, or 2.0 μM of Compound B and TPO exhibited reduced p-AKT levels of 11%, 44%, or 55%, respectively, and reduced S6 levels of 13%, 28%, or 48%, respectively, compared to those treated with vehicle.

### Example 2

### Expressions of PI3K Isoforms in Progenitor Cells from Myelofibrosis Patients

To examine the PI3K isoform expression, the CD34+ cells were isolated from peripheral blood from healthy individuals (subjects 1-2) and from myelofibrosis (MF) patients who had not received any prior treatment (i.e. naïve)(subjects 3-5), had chronically received ruxolitinib (subjects 6-10) or Compound A (N-(cyanomethyl)-4-[2-(4-morpholinoandino)pyrimidin-4-yl]benzamide)(subject 11-13).

The CD34⁺ (CD34⁺/CD3⁻/CD14⁻/CD19⁻/CD66⁻) cells were labeled and sorted by FACSAria (Beckman-Dickenson). The cell lysates were analyzed by Simple Western using Peggy (ProteinSimple) and AUC was plotted to quantify the levels of PI3K isoforms. Recombinant PI3K proteins were used as positive controls, and GAPDH was used to normalize isoform expression to total proteins.

The results of the study were summarized in Table 1. Among all samples (i.e. healthy individuals, untreated and treated MF patients), the levels of PI3Kδ were the highest among four isoforms.

TABLE 1
Expressions of PI3K isoforms in the CD34+ cells from
healthy individuals and myelofibrosis patients.

| Subject | PI3Kα | PI3Kβ | PI3Kδ | PI3Kγ |
|---|---|---|---|---|
| 1 | 0 | 4700 | 32580 | 320 |
| 2 | 0 | 8300 | 39260 | 0 |
| 3 | 0 | 36250 | 131240 | 2025 |
| 4 | 2800 | 21310 | 119520 | 1500 |
| 5 | 0 | 21340 | 65120 | 660 |
| 6 | 0 | 17870 | 41390 | 0 |
| 7 | 0 | 17350 | 51490 | 0 |
| 8 | 0 | 7740 | 41620 | 0 |
| 9 | 0 | 20680 | 37975 | 0 |
| 10 | 0 | 14610 | 68630 | 1550 |
| 11 | 0 | 12040 | 55050 | 1050 |
| 12 | 0 | 27180 | 73280 | 1540 |

### Example 3

### Effects of PI3K Inhibitors on Cellular Signaling in Progenitor Cells from Myelofibrosis Patients

PBMCs were isolated from whole blood of myelofibrosis (MF) patients who had not received treatments (i.e. naïve patients) or received ruxolitinib (i.e. rux-treated patients). The cells were treated with 0.02, 0.2, or 2.0 μM of Compound B or vehicle (0.1% DMSO in 0.1% FBS/RPMI) for 2 hours at 37° C. The cells were then fixed, permeabilized, and stained for FACS analysis. Antibodies specific to p-AKT Ser473 and pS6RP Ser235/236 were used to detect AKT phosphorylation (p-AKT) and S6RP phosphorylation (p-S6RP) in CD34⁺/CD3⁻/CD14⁻/CD19/CD66⁻ (BD Biosciences) gated cells using flow cytometry. The percentage of basal (i.e. untreated with TPO) AKT and S6RP phosphorylation were normalized to vehicle control. A two-tailed paired t-test (GraphPad Prism) was used to calculate p-values. Values of p<0.05 were considered significant.

All subjects had the JAK2V617F mutation. The basal levels of phosphorylation in the CD34⁺ (CD34⁺/CD3⁻/CD14⁻/CD19⁻/CD66⁻) cells without TPO activation are summarized in Table 2, and the p-values are summarized in Table 3. The results show that, compared to untreated progenitor MF cells, the cells treated with Compound B exhibited reduced levels of p-AKT (Table 2) and p-S6RP (data not shown). In addition, the cells treated with higher concentration of Compound B exhibited higher levels of reduction. Moreover, the reduced phosphorylation levels or PI3K signaling were observed in the cells from MF patients who had received or not received ruxolitinib. This suggests that Compound B caused a dose-dependent inhibition to PI3K signaling in naïve or treated MF patients.

TABLE 2
The normalized percentage of basal AKT phosphorylation in progenitor
cells isolated from naïve or rux-treated MF patients
treated with Compound B.

p-AKT

| Subject | 0 | 0.02 μM | 0.2 μM | 2 μM |
|---|---|---|---|---|
| Naïve-1 | 100 | 84 | 81 | 70 |
| Naïve-2 | 100 | NA | 72 | 47 |
| Naïve-3 | 100 | 99 | 64 | 55 |
| Naïve-4 | 100 | 102 | 127 | 96 |
| Naïve-5 | 100 | 83 | 75 | 66 |
| Naïve-6 | 100 | 88 | 76 | 66 |
| Rux-1 | 100 | 88 | 85 | 69 |
| Rux-2 | 100 | 89 | 78 | 77 |
| Rux-3 | 100 | 91 | 81 | 83 |
| Rux-4 | 100 | 89 | 82 | 84 |
| Rux-5 | 100 | 57 | 52 | 43 |
| Rux-6 | 100 | 96 | 87 | 98 |
| Rux-7 | 100 | 100 | 82 | 79 |

TABLE 3
The p-values of basal AKT and S6RP phosphorylation in
the progenitor cells isolated from naïve
or rux-treated MF patients treated with Compound B.

| | p-AKT | | | p-S6RP | | |
|---|---|---|---|---|---|---|
| | 0.02 | 0.2 | 2 | 0.02 | 0.2 | 2 |
| Subjects | μM | μM | μM | μM | μM | μM |
| Naïve | NS¹ | NS | 0.0047 | 0.0205 | 0.0129 | 0.0151 |
| Rux-treated | 0.005 | 0.0027 | 0.0099 | 0.08 | 0.0002 | 0.0001 |

¹NS: not significant

Also, PBMC cells from naïve or ruxolitinib treated patients were isolated and treated with Compound B and with TPO as described above. The percentage of TPO-activated AKT and S6RP phosphorylation were normalized to those of TPO-treated vehicle (“no TPO” values in Table 4). Results are summarized in Table 4, and the p-values are summarized in Table 5. Similar to those without TPO treatment, the cells treated with Compound B exhibited reduced levels of p-AKT and p-S6RP. Also, the inhibition to PI3K signaling was dose-dependent to Compound B.

TABLE 4
The normalized percentage of TPO-activated AKT and
S6RP phosphorylation in the progenitor cells from naïve
or rux-treated MF patients treated with Compound B.

| | | p-AKT | | | | | p-S6RP | | | |
|---|---|---|---|---|---|---|---|---|---|---|
| | No | | 0.02 | 0.2 | 2.0 | No | | 0.02 | 0.2 | 2.0 |
| Subject | TPO | 0 | μM | μM | μM | TPO | 0 | μM | μM | μM |
| Naïve-3 | 20 | 100 | 58 | 41 | 27 | 17 | 100 | 86 | 69 | 45 |
| Naïve-4 | 45 | 100 | 43 | 30 | 28 | 32 | 100 | 57 | 48 | 59 |
| Naïve-5 | 53 | 100 | 60 | 32 | 32 | 11 | 100 | 50 | 42 | 23 |
| Rux | 68 | 100 | 59 | 39 | 40 | 15 | 100 | 55 | 35 | 21 |
| Rux-3 | 53 | 100 | 60 | 52 | 29 | 27 | 100 | 79 | 56 | 45 |
| Rux-4 | 14 | 100 | 60 | 55 | 35 | 14 | 100 | 60 | 55 | 35 |
| Rux-5 | 54 | 100 | 78 | 57 | 39 | 6 | 100 | 59 | 50 | 36 |
| Rux-6 | 16 | 100 | 55 | 36 | 16 | 7 | 100 | 53 | 32 | 20 |
| Rux-7 | 13 | 100 | 57 | 33 | 16 | 5 | 100 | 62 | 44 | 31 |

TABLE 5
The p-values of TPO-activated AKT and S6RP phosphorylation
in MF progenitor cells treated with Compound B.

| | p-AKT | | | p-S6RP | | |
|---|---|---|---|---|---|---|
| | 0.02 | 0.2 | 2 | 0.02 | 0.2 | 2 |
| Subjects | μM | μM | μM | μM | μM | μM |
| Naïve | 0.013 | 0.003 | 0.0005 | NS¹ | 0.029 | 0.03 |
| Rux-treated | 0.0001 | 0.0001 | 0.0001 | 0.0002 | 0.0001 | 0.0001 |

¹NS: not significant

### Example 4

### Effects of Compounds C and D on AKT and S6PR Phosphorylation

Similar studies were conducted with PI3K inhibitors Compounds C and D. PBMC from MF patients had received ruxolitinib (rux) and MF patient had received Compound A. The cells were treated with Compounds C or D at 0, 20.0, 200.0, 2000.0 nM for 2 hours at 37° C. Cells were treated with TPO for 10 minutes. The percentage of basal p-AKT and p-S6RP levels were normalized to vehicle control and those of TPO-treated were normalized to TPO-treated vehicle control. The PI3Kδ inhibitors Compound C is referred by the chemical names of (S)-2-(1-((9H-purin-6-yl)amino)ethyl)-6-fluoro-3-phenylquinazolin-4(3H)-one.

Results showing their effects in basal (TPO-untreated) and TPO-treated cells are summarized in Table 6. Similar to Compound B, Compounds C and D inhibited the PI3Kδ signaling as shown by the reduced phosphorylation levels of AKT and S6RP in MF progenitor cells. Also, Compounds C and D inhibited p-AKT and p-S6RP in a dose dependent manner as higher concentrations of Compound C resulted in higher reduction in AKT/S6RP phosphorylation or PI3K signaling. Both compounds caused inhibition or reduction in the PI3K signaling or AKT/S6RP phosphorylation.

TABLE 6
The percentage of p-AKT and p-S6RP in basal and TPO-
treated MF progenitor cells treated with Compound C.

| | | Rux-treated Cells | | | | Compound A-treated Cells | | | |
|---|---|---|---|---|---|---|---|---|---|
| | | Basal | | TPO | | Basal | | TPO | |
| | | pAKT | pS6 | pAKT | pS6 | pAKT | pS6 | pAKT | pS6 |
| 0 | nM | 100 | 100 | 100 | 100 | 100 | 100 | 100 | 100 |
| 20 | nM | 88 | 65 | 65 | 84 | 89 | 82 | 65 | 66 |
| 200 | nM | 90 | 59 | 53 | 62 | 82 | 75 | 59 | 70 |
| 2000 | nM | 75 | 43 | 24 | 42 | 75 | 53 | 43 | 36 |

| | | | | | | | | |
|---|---|---|---|---|---|---|---|---|
| No TPO | NA² | NA | 40 | 29 | NA | NA | 16 | 9 |

²NA: not applicable

### Example 5

### Effects of PI3K Inhibitor and/or JAK Inhibitor in MF Progenitor Cells

In this example, effects of PI3K inhibitors and JAK inhibitors on cell growth and apoptosis were examined. To measure the effects on cell growth, PBMCs were isolated from the whole blood of MF patients had received chronic ruxolitinib. The cells were stained, and CD34+ cells (CD34⁺/CD3⁻/CD14⁻/CD19⁻/CD66⁻) were isolated via sorting using FACSAria. About 10,000 cells per 96-well plate were added in StemSpan SFEM II media containing StemSpan CC110 cytokine cocktail (STEMCELL technologies). The cells were treated with either 1.0 μM of Compound B, 0.5 μM of ruxolitinib, the combination of 1.0 μM of Compound B and 0.5 μM of ruxolitinib, or vehicle (0.1% DMSO). After 72 hours, cell growth was measured using CellTiter-Glo (Promega). Raw data from all subjects treated with Compound B and/or ruxolitinib, or vehicle were collected together and calculated for the p-values using two-tailed paired t-test (GraphPad).

As shown in Table 7, the cells treated with Compounds B and/or ruxolitinib exhibited reduced cell viability or cell growth. Higher percentage indicates more viable cells. The cells treated with both compounds had the highest inhibition effects. This suggests the combination of PI3K inhibitor (such as Compound B) and JAK inhibitor (such as ruxolitinib) resulted in increased cell inhibition. The p-values were calculated for each compound alone vs. the combination: p=0.0001 for compound B compared to the combination, and, p=0.0003 for ruxolitinib compared to the combination. Values of p<0.5 were significant.

TABLE 7
The percentage of viable cells in MF progenitor cells
treated with Compounds B and/or ruxolitinib.

| | | 1 μM | 0.5 μM | 1 μM Compound B + |
|---|---|---|---|---|
| Sample | Vehicle | Compound B | ruxolitinib | 0.5 μM ruxolitinib |
| 1 | 100 | 73 | 45 | 25 |
| 2 | 100 | 68 | 23 | 13 |
| 3 | 100 | 73 | 36 | 24 |
| 4 | 100 | 89 | 62 | 40 |
| 5 | 100 | 69 | 48 | 29 |
| 6 | 100 | 87 | 74 | 52 |
| 7 | 100 | 51 | 75 | 26 |
| 8 | 100 | 65 | 38 | 17 |
| 9 | 100 | 62 | 54 | 24 |

To measure apoptosis, PBMCs from MF patients who had received chronic ruxolitnib or Compound A were stained and isolated for CD34+ cells (CD34⁺/CD3⁻/CD14⁻/CD19⁻/CD66⁻) via sorting using FACSAria. About 10,000 cells per 96-well were plated in StemSpan SEEM II media containing StemSpan CC110 cytokine cocktail (STEMCELL Technologies). The cells either 1.0 μM of Compound B, 0.5 μM of ruxolitinib, the combination of 1.0 μM of Compound B and 0.5 μM of ruxolitinib, or vehicle. After 72 hours, the cell death or apoptosis was measured by labeling cells with 7-AAD/Annexin-V (GuavaNexin) followed by FACS analysis. The p-values were calculated for each compound alone vs. the combination: p=0.0001 for compound B compared to the combination and p=0.0001 for ruxolitinib compared to the combination. Values of p<0.5 were significant.

Table 8 summarizes the percentages of Annexin-V positive cells from the ruxolitinib-treated MF patients, and Table 9 summarizes the percentages of Annexin-V positive cells from the Compound A-treated patients (subjects 10-12 in Example 2). As Annexin-V labels apoptotic cells, higher percentage indicates more apoptotic cells, i.e. increased cell death. The results show that the cells (from the ruxolitinib-treated MF patients) treated with either Compound B or ruxolitinib exhibited induced apoptosis, and that the cells treated with both compounds exhibited the highest induction of apoptosis.

TABLE 8
The percentage of Annexin-V positive cells in the progenitor cells
from the ruxolitinib-treated MF patients treated with Compounds B
and/or ruxolitinib.

| | | 1 μM | 0.5 μM | 1 μM Compound B + |
|---|---|---|---|---|
| Sample | vehicle | Compound B | ruxolitinib | 0.5 μM ruxolitinib |
| 1 | 24 | 31 | 42 | 52 |
| 2 | 11 | 14 | 22 | 26 |
| 3 | 27 | 31 | 49 | 57 |
| 4 | 21 | 24 | 35 | 44 |
| 5 | 63 | 68 | 71 | 79 |
| 6 | 51 | 55 | 57 | 63 |
| 7 | 20 | 25 | 29 | 42 |
| 8 | 56 | 60 | 67 | 75 |

TABLE 9
The percentage of Annexin-V positive cells in the
progenitor cells from the Compound A-treated MF patients
treated with Compounds B and/or ruxolitinib.

| | p-AKT | | | | | p-S6RP | | | | |
|---|---|---|---|---|---|---|---|---|---|---|
| | No | | 0.02 | 0.2 | 2.0 | No | | 0.02 | 0.2 | 2.0 |
| Subject | TPO | 0 | μM | μM | μM | TPO | 0 | μM | μM | μM |
| 10 | 52 | 100 | 51 | 51 | 30 | 13 | 100 | 64 | 42 | 25 |
| 11 | 52 | 100 | 56 | 49 | 24 | 77 | 100 | 61 | 52 | 30 |
| 12 | 88 | 100 | 74 | 71 | 55 | 82 | 100 | 69 | 54 | 34 |

In other studies, the cells from MF patients are treated with Compounds B, C, or D in combination with Compound A. MF patients may be naïve (i.e. have not received any treatments) or have received JAK inhibitor such as ruxolitinib or Compound A. The cell viability and the apoptosis of the treated cells are measured as described above.

### Example 7

### Combination Treatment with PI3Kδ Inhibitor and JAK Inhibitor

This study evaluates the efficacy and safety of combination treatment of Compound B and ruxolitinib in patients having primary myelofibrosis, post-polycythemia or post-essential thrombocythemia myelofibrosis. The patients may have progressive or relapsed disease, or disease persistence on maximum clinically tolerated ruxolitinib therapy. The patients with progressive disease have: (i) appearance of a new splenomegaly that is palpable at least 5 cm below LCM, (ii) more than or equal to 100% increase in palpable distance, below LCM, for baseline splenomegaly of 5-10 cm, or (iii) about 50% increase in palpable distance, below LCM, for baseline splenomegaly of >10 cm. Also, the patients with relapsed disease have: (i) below criteria for at least CI after achieving CR, PR, or CI, or Loss of anemia response persisting for at least 1 month, or (ii) loss of spleen response persisting for at least 1 month. Also, disease persistence is defined as patients who are receiving FDA-approved JAK inhibitor therapy who meet the following criteria: relapsed disease, stable disease, or progressive disease with palpable splenomegaly (of >5 cm) that persists for 8 weeks up until the screening visit.

The patients are administered with ruxolitinib at a stable dose of 20, 15, or 5 mg (based on platelet count) orally twice daily for 8 weeks before being administered with 100 mg of Compound B orally twice daily in continuous 28 day cycles (1 cycle=28 days). After 2 cycles, patients may receive either 100 or 150 mg of Compound B orally twice daily. The patients continue to receive ruxolitinib, orally twice daily, at the same dose as pre-Compound B administration. The minimum duration of the study is 6 months.

Plasma concentration of Compound B is measured at trough (i.e., pre-dose) and peak (i.e., 1.5 hours post-dose) time points. At the end of each cycle, patients are evaluated at the end of each cycle for response rate, symptom burden, bone marrow fibrosis, and molecular responses. Response rate is defined as better than stable disease (including clinical improvement, partial improvement, or complete Improvement, spleen response, anemia response, symptoms response) according to criteria by International Working Group for Myelofibrosis Research and Treatment. The MF-associated symptomatic burden is determined by the Myeloproliferative Neoplasm Symptom Assessment Form, and bone marrow fibrosis is determined by European Fibrosis Scoring System. Blood samples are used to determine phosphorylation of the PI3K/AKT and other phosphorylated signaling intermediates (e.g., AKT, S6, STAT3, STATS, ERK, NFkB), genetic mutation (e.g. JAK2V617F), and levels of systemic cytokines and chemokines (e.g., IL-6, IL-1RA, IL-1B, IL-2, FGF, MIP1b, TNFα, CCL3, CCL4, CXCL12, CXCL13).

Similar studies are conducted to evaluate the efficacy and safety of combination treatment of Compound A with Compounds B, C, D, or E in patients having primary myelofibrosis, post-polycythemia or post-essential thrombocythemia myelofibrosis.

### Example 8

### Combination of PI3K Inhibitor with Anti-CD20 Antibodies

Obinutuzumab is a glycoengineered, type II, anti-CD20 antibody that induces cell death (Herter et al., Mol. Cancer Ther. 12:2031-42, 2013; Mossner et al. Blood 115:4393-402, 2010). Glycoengineering of obinutuzumab may increase the affinity for FcγRIII on innate immune effector cells, resulting in enhanced induction of antibody-dependent cellular cytotoxicity (ADCC) and phagocytosis (ADCP). Obinutuzumab is approved for first-line treatment of CLL patients in combination with chlorambucil in the US and EU, and is currently in pivotal clinical trials in indolent non-Hodgkin lymphoma (iNHL) and diffuse large B-cell lymphoma (DLBCL). Obinutuzumab may be administered intravenously at 100 mg on day 1, 900 mg on day 2, and 1000 mg on days 8 and 15 during cycle 1, followed by 1000 mg every 28 days during cycles 2-6; chlorambucil may be administered orally at 0.5 mg/kg on days 1 and 15 of each cycle. Ibrutinib is shown to interfere with the immune effector function and in vivo efficacy of rituximab in preclinical models (Kohrt et al., Blood 123:1957-60, 2014).

PI3K isoforms may play a role in immune effector cells and FcγR signaling. The effects of Compound B on the immune effector functions of obinutuzumab and rituximab in lymphoma cell lines were examined. To characterize the antibody-dependent cellular cytotoxicity (ADCC), PBMCs were isolated from healthy individuals with FcγRIIIa genotypes of 158F/F, 158F/V, or 158V/V (Leuko Paks from AllCells, Alameda, Calif.) using Ficoll density gradient centrifugation. NK cells were enriched using a negative-selection immunomagnetic enrichment kit (STEMCELL Technologies, Vancouver, British Columbia, Canada). Enriched NK cells and target cells WIL2-S, S-DHL-4, or Z-138 were separately pre-incubated for 1 hour with or without Compound B (1/2 dilutions from about 1 mM to about 1 nM). In the last 20 minutes of the pre-incubation, target cells were opsonized with or without rituximab or obinutuzumab (at 10 μg/mL, the saturating concentration with maximal ADCC) at indicated effector-target ratios (E:T). Palivizumab was used as an isotype control. NK and target cells were combined and incubated for 4 hours at 37° C. in 5% CO₂. To determine ADCC, lactose dehydrogenase (LDH) was measured using a cytotoxicity detection kit (Roche Applied Science, Indianapolis, Ind.). In some assays, antigen-independent cellular cytotoxicity (AICC) which represented spontaneous release by NK cell killing of target cells without antibodies were used as control.

LDH release assays were conducted at 4 hours using WIL2-S line as target and purified NK cells (E:T=10:1). The results (n=9) were normalized as % of maximum ADCC. As shown in Table 10, Compound B did not affect obinutuzumab-mediated ADCC. Similar results were observed for rituximab-medicated ADCC (data not shown). This differs from previous reports that ibrutinib, a BTK inhibitor, caused increased inhibition to rituximab-mediated ADCC compared to ibrutinib inhibition to obinutuzumab-mediated ADCC.

TABLE 10
Percentage of maximum obinutuzumab-mediated
ADCC with dose titration of Compound B.

| Com- | | | | | | | | | | |
|---|---|---|---|---|---|---|---|---|---|---|
| pound B | | Obinutuzumab (10 μg/mL) | | | | | | | | |
| 1000 | nM | 79 | 95 | 95 | 90 | 105 | 103 | 93 | 83 | 82 |
| 500 | nM | 83 | 94 | 92 | 96 | 106 | 104 | 94 | 92 | 79 |
| 250 | nM | 91 | 99 | 97 | 95 | 106 | 109 | 102 | 99 | 89 |
| 125 | nM | 87 | 104 | 97 | 97 | 111 | 110 | 105 | 94 | 84 |
| 62.5 | nM | 88 | 103 | 95 | 108 | 112 | 108 | 103 | 98 | 78 |
| 31.25 | nM | 85 | 102 | 107 | 96 | 107 | 107 | 107 | 95 | 82 |
| 15.63 | nM | 80 | 98 | 110 | 100 | 106 | 108 | 109 | 93 | 90 |
| 7.81 | nM | 89 | 98 | 113 | 105 | 109 | 107 | 107 | 94 | 90 |
| 3.91 | nM | 84 | 102 | 113 | 102 | 105 | 108 | 110 | 103 | 91 |
| 1.95 | nM | 89 | 100 | 125 | 100 | 104 | 106 | 112 | 107 | 100 |
| 0.98 | nM | 91 | 109 | 94 | 96 | 103 | 104 | 104 | 101 | 99 |

Also, as shown in Table 11, Compound B did not affect obinutuzumab-mediated ADCC in FcγRIIIa 158F/F or 158F/V genotypes (n=2 per genotype, LDH release assays at 4 hours using WIL2-S line as target and purified NK cells E:T=10:1; antibody concentration at 10 μg/mL). Compound B at 250 nM (the assay concentration similar to Cₘₐₓ of the clinical concentration) inhibited less than 10% of obinutuzumab-mediated ADCC in the FcγRIIIa 158V/V genotype. Moreover, as shown in Table 12, Compound B did not affect NK-mediated ADCC (LDH release assays at 4 hours using WIL2-S line as target and purified NK cells; antibody concentration at 10 μg/mL, n=3 at effector (NK cells) to target ratios (WIL2-S) varying from 1:1 to 10:1).

TABLE 11
Percentage of maximum obinutuzumab-mediated ADCC with dose
titration of Compound B in different FcγRIIIa genotypes.

| | | FcγRIIIa | | | | | | | | | | | |
|---|---|---|---|---|---|---|---|---|---|---|---|---|---|
| | | 158F/F | | | | 158F/V | | | | 158V/V | | | |
| Compound B | | rituximab | | Obinutuzumab | | rituximab | | obinutuzumab | | rituximab | | obinutuzumab | |
| 1000 | nM | 80 | 100 | 79 | 103 | 95 | 88 | 105 | 93 | 87 | 84 | 83 | 82 |
| 500 | nM | 84 | 105 | 83 | 104 | 95 | 99 | 106 | 94 | 91 | 96 | 92 | 79 |
| 250 | nM | 87 | 109 | 91 | 109 | 98 | 101 | 106 | 102 | 95 | 94 | 99 | 89 |
| 125 | nM | 87 | 108 | 87 | 110 | 105 | 102 | 111 | 105 | 95 | 98 | 94 | 84 |
| 62.5 | nM | 88 | 108 | 88 | 108 | 101 | 107 | 112 | 103 | 96 | 99 | 98 | 78 |
| 31.25 | nM | 86 | 109 | 85 | 107 | 100 | 104 | 107 | 107 | 94 | 94 | 95 | 82 |
| 15.63 | nM | 92 | 108 | 80 | 108 | 100 | 108 | 106 | 109 | 98 | 101 | 93 | 90 |
| 7.81 | nM | 91 | 108 | 89 | 107 | 104 | 110 | 109 | 107 | 100 | 100 | 94 | 90 |
| 3.91 | nM | 97 | 109 | 84 | 108 | 102 | 110 | 105 | 110 | 102 | 105 | 103 | 91 |
| 1.95 | nM | 103 | 111 | 89 | 106 | 105 | 111 | 104 | 112 | 105 | 104 | 107 | 100 |
| 0.98 | nM | 104 | 107 | 91 | 104 | 100 | 105 | 103 | 104 | 102 | 99 | 101 | 99 |

TABLE 12
Percentage of ADCC in NK cells treated with obinutuzumab, Palivizumab, or Compound
B at varying ratios of effector (NK cells) to target (WIL2-S) ratios.

| | | obinutuzumab | | | | | | Palivizumab | | | 500 nM | | | 50 nM | | | 5 nM | | |
|---|---|---|---|---|---|---|---|---|---|---|---|---|---|---|---|---|---|---|---|
| | E:T | ADCC | | | AICC* | | | ADCC | | | Compound B | | | Compound B | | | Compound B | | |
| S1 | 1:1 | 12 | 17 | 13 | 2 | 3 | 1 | 2 | 3 | 2 | 10 | 13 | 14 | 15 | 15 | 12 | 16 | 16 | 15 |
| | 3:1 | 43 | 44 | 43 | 5 | 5 | 5 | 5 | 7 | 6 | 39 | 35 | 38 | 42 | 42 | 42 | 44 | 44 | 39 |
| | 10:1 | 74 | 74 | 71 | 16 | 15 | 15 | 16 | 17 | 17 | 66 | 70 | 71 | 69 | 74 | 73 | 72 | 74 | 73 |
| | 30:1 | 81 | 81 | 72 | 31 | 31 | 29 | 37 | 36 | 31 | 76 | 79 | 77 | 76 | 81 | 80 | 81 | 82 | 79 |
| S2 | 1:1 | 31 | 27 | 23 | 4 | 3 | 2 | 4 | 4 | 3 | 26 | 24 | 25 | 30 | 25 | 22 | 28 | 33 | 30 |
| | 3:1 | 73 | 73 | 49 | 12 | 10 | 9 | 11 | 10 | 8 | 50 | 53 | 50 | 54 | 58 | 50 | 58 | 56 | 53 |
| | 10:1 | 74 | 73 | 69 | 30 | 28 | 27 | 27 | 31 | 22 | 67 | 73 | 71 | 72 | 74 | 69 | 72 | 75 | 73 |
| | 30:1 | 80 | 84 | 69 | 37 | 36 | 34 | 39 | 34 | 34 | 72 | 76 | 75 | 76 | 79 | 75 | 76 | 80 | 77 |
| S3 | 1:1 | 17 | 13 | 14 | 4 | 0 | 4 | 4 | −1 | 3 | 14 | 10 | 15 | 18 | 14 | 18 | 16 | 14 | 17 |
| | 3:1 | 39 | 35 | 36 | 9 | 4 | 7 | 8 | 4 | 6 | 36 | 34 | 36 | 39 | 37 | 44 | 49 | 46 | 44 |
| | 10:1 | 73 | 69 | 67 | 18 | 14 | 17 | 18 | 15 | 17 | 71 | 73 | 73 | 77 | 76 | 77 | 79 | 75 | 75 |
| | 30:1 | 89 | 88 | 84 | 28 | 13 | 27 | 29 | 24 | 25 | 81 | 81 | 82 | 85 | 84 | 85 | 85 | 84 | 84 |

*AICC: antigen-independent cellular cytotoxicity

The antibody potency and NK-cell expression of CD107a and CD16 in cells treated with obinutuzumab (0.01, 0.1, 1, 10, 100, or 1000 ng/mL) alone or combined with Compound B (256 nM), rituximab (0.01, 0.1, 1, 10, 100, or 1000 ng/mL) alone or combined with Compound B (256 nM) were determined Compound B at 256 nM may correspond to maximal average plasma concentration (Cₘₐₓ), which was adjusted for protein binding, in a patient administered with Compound B at the clinical dose of 150 mg, twice a day. The results showed that the presence of Compound B may reduce the potency of both anti-CD20 antibodies by 0 to 15% (data not shown). Additionally, Compound B inhibited NK-cell degranulation as measured by surface expression of CD107a in 2 of 3 samples (data not shown).

Next, antibody-dependent cellular phagocytosis (ADCP) was characterized. The CD14⁺ negatively selected monocytes (Astarte Biologics, Bothell, Wash.) were cultured in Gibco AIM V Medium CTS (Life Technologies, Grand Island, N.Y.) with macrophage colony-stimulating factor (PeproTech, Rocky Hill, N.J.) at 60 ng/mL. At Day 6 or 7, monocyte-derived macrophages were washed and plated with polarizing cytokines. For differentiation to M1 macrophages, cells were plated with interferon-γ 100 ng/mL (R&D Systems, Minneapolis, Minn.) and lipopolysaccharides from E. coli 055:B5 100 ng/mL (Sigma-Aldrich, St Louis, Mo.) for 24 hours. For differentiation to M2c macrophages, cells were plated in interleukin-10 10 ng/mL (R&D Systems) for 48 hours. Compound B titration was added to the plated macrophages and incubated at 37° C. for about 1 hour. Obinutuzumab or rituximab was then added to the cultures in 50 μL at a final concentration of 150 ng/mL. WIL2-S target cells labeled with Molecular Probes CellTracker Red CMTPX (Life Technologies) were added at an E:T of 3:1. The co-cultures were incubated for 2 hours at 37° C. Cells were then stained with pooled FITC anti-CD14 (Becton, Dickinson and Company, Franklin Lakes, N.J.) and FITC anti-CD11b (eBioscience, San Diego, Calif.), harvested from 96-well plates with Accutase (Merck Millipore, Darmstadt, Germany) and vigorous pipetting, and analyzed on an LSR II flow cytometer (Becton, Dickinson and Company). Double-positive cells (FITC+CellTracker Red) represented phagocytized target cells and the levels of phagocytosis were calculated as % double-positive cells/% double positive cells+% target cells alone×100.

Results showed that less than 30% inhibition of ADCP was observed in the treatment with Compound B at 256 nM using polarized macrophages. Further, whole blood (WB) autologous B-cell depletion and cell-death induction assays were conducted as described in Mossner et al., Blood 115:4393-402, 2010. For cell death assay: Ri-1 DLBCL cells were seeded at 15,000 cells/well in 96-well plates. Cells were preincubated with Compound B or DMSO for 1 hour before the addition of antibody. Plates were incubated at 37° C. in a humidified CO₂ chamber for 3 days. Cells were washed once with PBS and treated with Accutase for 15 minutes before being stained with Guava Nexin® reagent and analyzed on the Guava EasyCyte flow cytometer (Merck Millipore).

After 3 days of incubation, cell death was assessed by Annexin V/7AAD staining. As shown in Table 13, the combination of Compound B and obinutuzumab increased cell death compared to each agent alone (p<0.05 at all concentrations).

TABLE 13
Percentage of total Annexin V⁺ cells treated with Compound
B alone or in combination with obinutuzumab or palivizumab.

| | Palivizumab | | | | | | | | | | | | | | | | | |
|---|---|---|---|---|---|---|---|---|---|---|---|---|---|---|---|---|---|---|
| | 0 | | 0 | | | | 0 | | | | 0 | | | | 10 μg/mL | | | |
| | Obinutuzumab | | | | | | | | | | | | | | | | | |
| | 0 | | 0.1 μg/mL | | | | 1 μg/mL | | | | 10 μg/mL | | | | 0 | | | |
| 0 nM | 17 | 23 | 30 | 36 | 35 | 37 | 50 | 49 | 46 | 45 | 54 | 49 | 54 | 56 | 13 | 19 | 14 | 21 |
| Compound B | | | | | | | | | | | | | | | | | | |
| 100 nM | 28 | 27 | 39 | 40 | 41 | 43 | 56 | 60 | 55 | 53 | 63 | 59 | 58 | 62 | 27 | 25 | 29 | 26 |
| Compound B | | | | | | | | | | | | | | | | | | |
| 300 nM | 31 | 33 | 40 | 41 | 39 | 41 | 59 | 61 | 61 | 60 | 66 | 63 | 66 | 62 | 29 | 29 | 31 | 30 |
| Compound B | | | | | | | | | | | | | | | | | | |
| 600 nM | 35 | 37 | 40 | 42 | 45 | 44 | 61 | 61 | 61 | 59 | 63 | 68 | 69 | 71 | 33 | 32 | 32 | 33 |
| Compound B | | | | | | | | | | | | | | | | | | |

The results of WB autologous B-cell depletion assay were summarized in Tables 14 and 15. The percentage of deplete autologous B cells in a whole blood assay may represent the antibody potency.

TABLE 14
Percentage of B-cell depletion in WB treated with obinutuzumab
alone or in combination with Compound B.

| | | | | obinutuzumab + | | obinutuzumab + | | obinutuzumab + | |
|---|---|---|---|---|---|---|---|---|---|
| | conc* | obinutuzumab | | 4200 nM Compound B | | 760 nM Compound B | | 100 nM Compound B | |
| | (ng/mL) | % | SD\hat{ } | % | SD\hat{ } | % | SD\hat{ } | % | SD\hat{ } |
| S1 | 1000 | 56 | 2 | 44 | 2 | 48 | 3 | NA | NA |
| | 200 | 50 | 4 | 37 | 2 | 39 | 1 | NA | NA |
| | 40 | 43 | 4 | 31 | 5 | 34 | 3 | NA | NA |
| | 8 | 34 | 5 | 15 | 3 | 25 | 1 | NA | NA |
| | 2 | 8 | 3 | 7 | 2 | 11 | 5 | NA | NA |
| | 0.3 | 1 | 3 | 3 | 6 | 9 | 2 | NA | NA |
| | 0.06 | −4 | 5 | 7 | 2 | 9 | 3 | NA | NA |
| S2 | 1000 | 52 | 1 | 41 | 0 | 50 | 2 | NA | NA |
| | 200 | 50 | 2 | 37 | 3 | 49 | 1 | NA | NA |
| | 40 | 49 | 2 | 42 | 3 | 47 | 3 | NA | NA |
| | 8 | 38 | 1 | 24 | 3 | 34 | 2 | NA | NA |
| | 2 | 13 | 3 | 15 | 3 | 18 | 2 | NA | NA |
| | 0.3 | 0 | 3 | 8 | 3 | 10 | 2 | NA | NA |
| | 0.06 | −2 | 1 | 13 | 5 | 8 | 4 | NA | NA |
| S3 | 1000 | 63 | 4 | 71 | 1 | 73 | 3 | 69 | 2 |
| | 200 | 50 | 0 | 70 | 2 | 67 | 2 | 64 | 2 |
| | 40 | 60 | 3 | 68 | 0 | 71 | 2 | 68 | 1 |
| | 8 | 55 | 5 | 61 | 2 | 65 | 2 | 58 | 4 |
| | 2 | 38 | 6 | 31 | 1 | 43 | 2 | 41 | 1 |
| | 0.3 | 17 | 5 | 8 | 2 | 15 | 4 | 14 | 5 |
| | 0.06 | 0 | 4 | 0 | 5 | 4 | 3 | 3 | 3 |
| S4 | 1000 | 71 | 1 | 68 | 0 | 68 | 1 | 69 | 1 |
| | 200 | 69 | 2 | 61 | 2 | 62 | 2 | 63 | 2 |
| | 40 | 65 | 3 | 51 | 1 | 55 | 4 | 59 | 4 |
| | 8 | 45 | 1 | 30 | 1 | 37 | 2 | 41 | 2 |
| | 2 | 16 | 2 | 15 | 0 | 13 | 1 | 15 | 1 |
| | 0.3 | 1 | 2 | 7 | 2 | 4 | 2 | 5 | 3 |
| | 0.06 | 0 | 2 | 8 | 1 | 3 | 2 | 4 | 3 |

*conc: final concentration of obinutuzumab
\hat{ }SD: standard deviation

TABLE 15
Percentage of B-cell depletion WB treated with Compound
B alone or in combination with obinutuzumab or rituximab.

| | | Obinutuzumab* + | | Rituximab* + | | | |
|---|---|---|---|---|---|---|---|
| | Compound B | Compound B | | Compound B | | Compound B | |
| | (nM) | % | SD\hat{ } | % | SD\hat{ } | % | SD\hat{ } |
| S1 | 4200 | 51 | 3 | −15 | 2 | 5 | 6 |
| | 760 | 51 | 3 | −7 | 5 | −1 | 2 |
| | 100 | 53 | 3 | 3 | 2 | 5 | 4 |
| | 0 | 57 | 1 | 10 | 7 | 2 | 3 |
| S2 | 4200 | 68 | 2 | 21 | 2 | 0 | 3 |
| | 760 | 70 | 4 | 30 | 4 | −1 | 4 |
| | 100 | 72 | 2 | 33 | 4 | −1 | 1 |
| | 0 | 71 | 1 | 41 | 3 | −1 | 1 |
| S3 | 4200 | 66 | 2 | 14 | 0 | 1 | 2 |
| | 760 | 65 | 1 | 21 | 3 | −4 | 2 |
| | 100 | 65 | 5 | 26 | 4 | −3 | 3 |
| | 0 | 65 | 2 | 28 | 3 | −4 | 2 |

\hat{ }SD: standard deviation
*obinutuzumab or rituximab at 10 μg/mL

These results suggest that PI3K-δ inhibition by Compound B at clinical concentrations may not affect or inhibit the immune effector function of obinutuzumab and rituximab, and that Compound B did not inhibit ADCC caused by saturating concentration of obinutuzumab and rituximab. Also, the results indicate that the combination of Compound B and obinutuzumab increased cell death compared with each agent separately. This indicates that the combination of Compound B and obinutuzumab may provide additional benefits in therapeutic treatments.

All of the U.S. patents, U.S. patent application publications, U.S. patent applications, foreign patents, foreign patent applications and non-patent publications referred to in this specification are incorporated herein by reference, in their entirety to the extent not inconsistent with the present description.

From the foregoing it will be appreciated that, although specific embodiments of the invention have been described herein for purposes of illustration, various modifications may be made without deviating from the spirit and scope of the invention.

What is claimed is:

1. A method for treating a myeloproliferative disorder, comprising administering to a patient a therapeutic effective amount of JAK inhibitor and a therapeutic effective amount of PI3K inhibitor.

2. The method of claim 2, wherein the JAK inhibitor is a JAK2 inhibitor selected from the group consisting of ruxolitinib or N-(cyanomethyl)-4-[2-(4-morpholinoanilino)pyrimidin-4-yl]benzamide; or a pharmaceutically acceptable salt thereof.

3. The method of claim 1 or 2, wherein the PI3K inhibitor is selected from the group of XL147, BKM120, GDC-0941, BAY80-6946, PX-866, CH5132799, XL756, BEZ235, and GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, ZSTK474, AS252424, TGX221, TG100115, IC87114, (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2-(1-((9H-purin-6-yl)amino)ethyl)-3-(2,6-difluorophenyl)quinazolin-4(3H)-one, (S)-4-amino-6-((1-(5-chloro-4-oxo-3-phenyl-3,4-dihydroquinazolin-2-yl)ethyl)amino)pyrimidine-5-carbonitrile; or a pharmaceutically acceptable salt thereof.

4. The method of any of claims 1-3, wherein the PI3K inhibitor is a PI3Kδ inhibitor selected from the group consisting of (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2-(1-((9H-purin-6-yl)amino)ethyl)-3-(2,6-difluorophenyl)quinazolin-4(3H)-one, (S)-4-amino-6-((1-(5-chloro-4-oxo-3-phenyl-3,4-dihydroquinazolin-2-yl)ethyl)amino)pyrimidine-5-carbonitrile; or a pharmaceutically acceptable salt thereof.

5. A method for treating cancer, comprising administering to a patient a therapeutic effective amount of an anti-CD20 antibody and a therapeutic effective amount of PI3K inhibitor.

6. The method of claim 5, wherein the anti-CD20 antibody is obinutuzumab.

7. The method of claim 5 or 6, wherein the PI3K inhibitor is selected from the group of XL147, BKM120, GDC-0941, BAY80-6946, PX-866, CH5132799, XL756, BEZ235, and GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, ZSTK474, AS252424, TGX221, TG100115, IC87114, (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2-(1-((9H-purin-6-yl)amino)ethyl)-3-(2,6-difluorophenyl)quinazolin-4(3H)-one, and (S)-4-amino-6-((1-(5-chloro-4-oxo-3-phenyl-3,4-dihydroquinazolin-2-yl)ethyl)amino)pyrimidine-5-carbonitrile; or a pharmaceutically acceptable salt thereof.

8. The method of any one of claims 5-7, wherein the PI3K inhibitor is (S)-2-(1-((9H-purin-6-yl)amino)propyl)-5-fluoro-3-phenylquinazolin-4(3H)-one, or a pharmaceutically acceptable salt thereof.

9. The method of any one of claims 5-8, wherein the PI3K inhibitor is administered at a dose between 100 mg and 500 mg.

10. The method of any one of claims 5-9, wherein the PI3K inhibitor is administered at a dose of 150 mg twice a day.

11. The method of any one of claims 5-10, wherein the administration of the anti-CD antibody is prior, concurrent, or subsequent to the administration of the PI3K inhibitor.

12. The method of any one of claims 5-11, wherein the PI3K inhibitor is administered orally.

13. The method of any one of claims 5-12, wherein the anti-CD20 antibody is administered intravenously.

14. A method for treating a human, who has or is suspected of having a cancer, comprising administering to the human an effective amount of Compound B 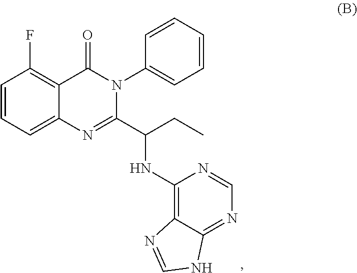 [SMILES: CCC(Nc1ncnc2[nH]cnc12)c1nc2cccc(F)c2c(=O)n1-c1ccccc1]
or a pharmaceutically acceptable salt thereof, and an effective amount of obinutuzumab.

15. The method of claim 14, wherein the Compound B or a pharmaceutically acceptable salt thereof is predominantly the (S)-enantiomer.

16. The method of claim 14 or 15, wherein:
Compound B or a pharmaceutically acceptable salt thereof is present in a pharmaceutical composition comprising Compound B or a pharmaceutically acceptable salt thereof, and at least one pharmaceutically acceptable vehicle; and
obinutuzumab is present in a pharmaceutical composition comprising obinutuzumab, and at least one pharmaceutically acceptable vehicle.

17. The method of any one of claims 14-16, wherein the human who has cancer is (i) refractory to at least one chemotherapy treatment, or (ii) is in relapse after treatment with chemotherapy, or a combination thereof.

18. The method of any one of claims 14-17, wherein the human has not previously been treated for the cancer.

19. The method of any one of claims 14-18, wherein the human has not previously been treated for chronic lymphocytic leukemia.

20. The method of any one of claims 14-19, wherein the cancer is leukemia, lymphoma, or multiple myeloma.

21. The method of any one of claims 14-20, wherein the cancer is selected from Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), B-cell ALL, acute myeloid leukemia (AML), chronic lymphocytic leukemia (CLL), small lymphocytic lymphoma (SLL), myelodysplastic syndrome (MDS), myeloproliferative disease (MPD), mantle cell lymphoma (MCL), follicular lymphoma (FL), Waldestrom's macroglobulinemia (WM), T-cell lymphoma, B-cell lymphoma, diffuse large B-cell lymphoma (DLBCL), marginal zone lymphoma (MZL), and minimal residual disease (MRD).

22. The method of any one of claims 14-21, wherein the cancer is selected from indolent non-Hodgkin's lymphoma (iNHL), chronic lymphocytic leukemia (CLL), and diffuse large B-cell lymphoma (DLBCL).

23. A method for decreasing cell viability, decreasing proliferation, or increasing apoptosis, comprising contacting cells with an effective amount of an anti-CD20 antibody and an effective amount of PI3K inhibitor.

24. The method of claim 23, wherein the anti-CD20 antibody is obinutuzumab.

25. The method of claim 23 or 24, wherein the PI3K inhibitor is selected from the group of XL147, BKM120, GDC-0941, BAY80-6946, PX-866, CH5132799, XL756, BEZ235, and GDC-0980, wortmannin, LY294002, PI3K II, TGR-1202, AMG-319, GSK2269557, X-339, X-414, RP5090, KAR4141, XL499, OXY111A, IPI-145, IPI-443, GSK2636771, BAY 10824391, buparlisib, BYL719, RG7604, MLN1117, WX-037, AEZS-129, PA799, ZSTK474, AS252424, TGX221, TG100115, IC87114, (S)-2-(1-((9H-purin-6-yl)amino)propyl)-5-fluoro-3-phenylquinazolin-4(3H)-one, (S)-2-(1-((9H-purin-6-yl)amino)ethyl)-6-fluoro-3-phenylquinazolin-4(3H)-one, (S)-2-(1-((9H-purin-6-yl)amino)ethyl)-3-(2,6-difluorophenyl)quinazolin-4(3H)-one, and (S)-4-amino-6-((1-(5-chloro-4-oxo-3-phenyl-3,4-dihydroquinazolin-2-yl)ethyl)amino)pyrimidine-5-carbonitrile; or a pharmaceutically acceptable salt thereof.

26. The method of any one of claims 23-25, wherein the cancer is selected from Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), B-cell ALL, acute myeloid leukemia (AML), chronic lymphocytic leukemia (CLL), small lymphocytic lymphoma (SLL), myelodysplastic syndrome (MDS), myeloproliferative disease (MPD), mantle cell lymphoma (MCL), follicular lymphoma (FL), Waldestrom's macroglobulinemia (WM), T-cell lymphoma, B-cell lymphoma, diffuse large B-cell lymphoma (DLBCL), marginal zone lymphoma (MZL), and minimal residual disease (MRD).

27. A pharmaceutical composition comprising a therapeutically effective amount of an anti-CD20 antibody, a therapeutically effective amount of PI3K inhibitor, and a pharmaceutically acceptable excipient.

28. A kit comprising a pharmaceutical composition and a label, wherein the pharmaceutical composition comprising a therapeutically effective amount of JAK inhibitor, a therapeutically effective amount of PI3K inhibitor, and a pharmaceutically acceptable excipient.

29. A kit comprising:
(i) a pharmaceutical composition comprising Compound B 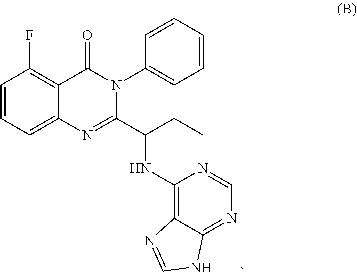 [SMILES: CCC(Nc1ncnc2[nH]cnc12)c1nc2cccc(F)c2c(=O)n1-c1ccccc1]
or a pharmaceutically acceptable salt thereof, and at least one pharmaceutically acceptable vehicle; and
(ii) a pharmaceutical composition comprising obinutuzumab, and at least one pharmaceutically acceptable vehicle.

30. The kit of claim 29, further comprising: a package insert containing instructions for use of the pharmaceutical compositions in treating a cancer.

31. The kit of claim 29 or 30, wherein the pharmaceutical composition comprising Compound B is a tablet.

32. The kit of claim 30 or 31, wherein the cancer is selected from Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), B-cell ALL, acute myeloid leukemia (AML), chronic lymphocytic leukemia (CLL), small lymphocytic lymphoma (SLL), myelodysplastic syndrome (MDS), myeloproliferative disease (MPD), mantle cell lymphoma (MCL), follicular lymphoma (FL), Waldestrom's macroglobulinemia (WM), T-cell lymphoma, B-cell lymphoma, diffuse large B-cell lymphoma (DLBCL), marginal zone lymphoma (MZL), and minimal residual disease (MRD).

33. An article of manufacture comprising:
(i) a unit dosage form of Compound B 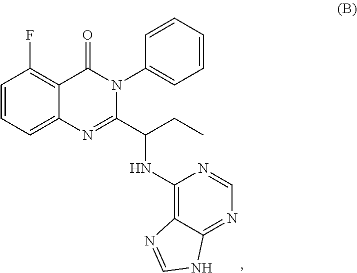 [SMILES: CCC(Nc1ncnc2[nH]cnc12)c1nc2cccc(F)c2c(=O)n1-c1ccccc1]
or a pharmaceutically acceptable salt thereof, and at least one pharmaceutically acceptable vehicle;
(ii) a unit dosage form of obinutuzumab; and at least one pharmaceutically acceptable vehicle; and
(iii) a label containing instructions for use of Compound B, or pharmaceutically acceptable salts thereof, and obinutuzumab, in treating cancer.

34. The article of manufacture of claim 33, wherein each unit dosage form is a tablet.

35. The article of manufacture of claim 33 or 34, wherein the cancer is selected from Burkitt's lymphoma, Hodgkin's lymphoma, non-Hodgkin's lymphoma (NHL), indolent non-Hodgkin's lymphoma (iNHL), refractory iNHL, multiple myeloma (MM), chronic myeloid leukemia (CML), acute lymphocytic leukemia (ALL), B-cell ALL, acute myeloid leukemia (AML), chronic lymphocytic leukemia (CLL), small lymphocytic lymphoma (SLL), myelodysplastic syndrome (MDS), myeloproliferative disease (MPD), mantle cell lymphoma (MCL), follicular lymphoma (FL), Waldestrom's macroglobulinemia (WM), T-cell lymphoma, B-cell lymphoma, diffuse large B-cell lymphoma (DLBCL), marginal zone lymphoma (MZL), and minimal residual disease (MRD).
